SEQUENCE LISTING (52 sequences)

SEQ ID NO: 1 - protein, 332 aa, Neisseria meningitidis
  MISC_FEATURE (1)..(332): lgtA, -1,3-N-acetyl-glucosaminyltransferase, GeneBank ID WP_033911473.1
MQPLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRPEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTLPAGAWLDFAADGRMRRLFTLRQYFGILHRLLKNR

SEQ ID NO: 2 - protein, 334 aa, Pasteurella multocida
  MISC_FEATURE (1)..(334): pmnagT, -1,3-N-acetylglucosaminyl-transferase, GeneBankID WP_014390683.1
MENKPLVSVLICAYNVEKYIEECINAVINQTYKNLEIIIVNDGSSDNTYFLLKKLAEKDNRIKILNFNNHIGIISALNEGLKEIAGEYIARTDSDDITKPDWIEKILTCMQNDPKIIAMGSYLTVLSEENNGSVLANHHKNKVEWKNPLEHKDIVEKMLFGNPIHNNSMVMRSEIYTKYHLIYDPDYHYAEDYKFWLEVSRIGKLANYPESLVYYRLHRNQTSSIHNSQQEINGKKLRLQALNYYLKDLGIDYQLPEKFLFKDIALLQEIFYERGMFRENIIRRIIYECYLSLGEYNYKDIYYFLINKNNFLSIKDKFKIIKKYLRPDKYSSTY

SEQ ID NO: 3 - protein, 330 aa, Haemophilus ducreyi
  MISC_FEATURE (1)..(330): HD9466, glycosyltransferase family 2 protein, GeneBank ID WP_010944479.1
MTTLVSVLICAYNVEKYIDECLNAVIAQTYKNLEIIVVNDGSTDGTLAKLRQFEAKDPRVKIIDNIVNQGTSKSLNIGIQYCQGEIIARTDSDDIVDIHWIETLMRELDNSPETIAISAYLEFLAEKGNGSKLSRSRKHGKNAENPISSEAISQRMLFGNPVHNNVALVRRKVFSEYGLRFDPDYIHAEDYKFWFEVSKLGKMRTYPKALVKYRLHATQVSSAYNQKQRSIAKKIKREAISHYLQQYGIQLPEKLTIHDLFSIFSPQIELSLTVANKQELFWSLATSLSEYHFRDLLKIYSLDIFHQLSFKYKKRIFRKFLLPNRYPSVI

SEQ ID NO: 4 - protein, 439 aa, Artificial Sequence
  galTK, -1,3-galactosyltransferase, homologous to GeneBank ID BD182026.1
MISVYIISLKESQRRLDTEKLVLESNEKFKGRCVFQIFDAISPKHEDFEKFVQELYDSSSLLKSDWFHSDYCYQELLPQEFGCYLSHYLLWKECVKLNQPVVILEDDVALESNFMQALEDCLKSPFDFVRLYGHYWGGHKTNLCALPVYTETEEAEASIEKTPIENYEVTSPPPPNPTRDTQQDFITETQQDPKELSEPCKIAPQKISFNQVVFKKIKRKLNRFIGSILARTEVYKNIVAKYDDLTTKYDDLTTKYDDLTTKYDDLTTKYDDLNKNIAEKYDELMGKYESLLAKEVNIKETFWESRADSEKEALFLDHFYLTSVYVATTAGYYLTPKGAKTFIEATERFKIIEPVDMFINNPTYHDIANFTYVPCPVSLNKHAFNSTIQNAKKPDISLKPPKKSYFDNLFYHKFNARKCLKAFNKYSKQYAPLKTPKEV

SEQ ID NO: 5 - protein, 262 aa, Chromobacterium violaceum
  MISC_FEATURE (1)..(262): cvb3galT, -1,3-galactosyltransferase, GeneBank ID WP_080969100.1
MDTIMIKRPLVSVILPVNKNNPHLEEAIQSIKNQTYKELELIIIANNCEDNFYSLLLKYQDQKTKIIRTSIKYLPFSLNLGVHLSQGEYIARMDSDDISVLDRIEKQVKRFLNTPELSILGSNVEYINEASESIGYSNYPLDHSSIVNSFPFRCNLAHPTIMVKKEVITTLGGYMYGSLSEDYDLWIRASRHGNFKFSNIDEPLLKYRIHKGQATNKSNAYNIFAFDSSLKIREFLLNGNVQYLLGAARGFFAFLYVRFIKK

SEQ ID NO: 6 - protein, 302 aa, Artificial Sequence
  FutC, -1,2-fucosyltransferase, homolouge to GeneBank ID WP_080473865.1
MAFKVVQICGGLGNQMFQYAFAKSLQKHSNTPVLLDITSFDWSDRKMQLELFPIDLPYASAKEIAIAKMQHLPKLVRDALKCMGFDRVSQEIVFEYEPKLLKPSRLTYFFGYFQDPRYFDAISPLIKQTFTLPPPPENNKNNNKKEEEYQCKLSLILAAKNSVFVHIRRGDYVGIGCQLGIDYQKKALEYMAKRVPNMELFVFCEDLEFTQNLDLGYPFMDMTTRDKEEEAYWDMLLMQSCQHGIIANSTYSWWAAYLIENPEKIIIGPKHWLFGHENILCKEWVKIESHFEVKSQKYNALG

SEQ ID NO: 7 - protein, 292 aa, Methylobacter tundripaludum
  MISC_FEATURE (1)..(292): Mtun, -1,2-fucosyltransferase, GeneBank ID WP_031437198.1
MVITHLIGGLGNQMFQYAAGRAVSLERGVSLSLDISGFANYGLHQGFELQRIFNCTAEIANEADVRGILGWQSSPRIRQLLSRQNMAIFRREGFVVEPHFHYWQGIKSVPRDCYLTGYWQSEQYFLEAAAQIRADFTFKLPLDNQNIELAKQINAVNAVSLHVRRGDYANTPETTATHGLCSLDYYRVAIRHIAEQVQQPHFFVFSDDIAWVKNNLSIDFPCQYVDHNQGAESYNDMRLMSMCRHHIIANSSFSWWGAWLNPNVNKIVVAPSRWFAKQTDVRDLLPQGWIKQ

SEQ ID NO: 8 - protein, 292 aa, Sulfuriflexus mobilis
  MISC_FEATURE (1)..(292): Smob, -1,2-fucosyltransferase, GeneBank ID WP_126455392.1
MIISQIIGGLGNQMFQYAAGRALSLVRGQPLLLDVTGFAGYGLHQGFELQRVFDCPIGIATEEDVRGILGWQFSAGIRRIVARPGMAAFRRKGFIVEPHFHYWPEIKNVPRDCYLLGYWQSERYFRAATADIRADFSFKSPLVNRNAETAAQIDQVNAISLHMRRGDYVNNPKTSATHGLCSLDYYQAAIKFVSERVEEPFFFIFSDDIAWVKANLKLDFPCQYVDHNHGAESFNDMHLMSLCQHHIIANSSFSWWGAWLNSDPKKIVLAPKKWFANKNNIKDLFPPGWVSL

SEQ ID NO: 9 - DNA, 203 bp, Artificial Sequence
  PmglB_70UTR, variant of E. coli promoter for mglBAC galactose/methyl-galactosidade transporter
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaggaggaaacagct

SEQ ID NO: 10 - DNA, 203 bp, Artificial Sequence
  PmglB_70UTR_SD4, variant of E. coli promoter for mglBAC; galactose/methyl-galactosidade transporter
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaactaggaaacagct

SEQ ID NO: 11 - DNA, 152 bp, Klebsiella pneumoniae
  misc_feature (1)..(152): Promoter for scrYA sucrose genes
ggttaacggcccactttgctggcgacatcacaattcttaaaccggtttagcaatttttattttcaccgcgttaccgacatgtttaccatatcaactaaaccggtttagcaaacattagcacactcactgatttacctttggatgtcaccaac

SEQ ID NO: 12 - DNA, 291 bp, Artificial Sequence
  PgatY_70UTR, variant of E. coli promoter for gatYZABCD; tagatose-1,6-bisP aldolase
cggcaacctatgcctgatgcgacgctgaagcgtcttatcatgcctacatagcactgccacgtatgtttacaccgcatccggcataaaaacacgcgcactttgctacggcttccctatcgggaggccgtttttttgcctttcactcctcgaataattttcatattgtcgtttttgtgatcgttatctcgatatttaaaaacaaataatttcattatattttgtgcctacaagcatcgtggaggtccgtgactttcacgcatacaacaaacattaaccaaggaggaaacagct

SEQ ID NO: 13 - DNA, 300 bp, Escherichia coli
  misc_feature (1)..(300): PglpF, E. Coli promoter sequence of glpFKX operon
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaggaggaaacagct

SEQ ID NO: 14 - DNA, 300 bp, Artificial Sequence
  PglpF_SD1, variant of PglpF E. Coli promoter sequence of glpFKX operon
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaattcgaaacagct

SEQ ID NO: 15 - DNA, 300 bp, Artificial Sequence
  PglpF_SD10, variant of PglpF E. Coli promoter sequence of glpFKX operon
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaactgagaaacagct

SEQ ID NO: 16 - DNA, 300 bp, Artificial Sequence
  PglpF_SD2, variant of PglpF E. Coli promoter sequence of glpFKX operon
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagcgcaaaacagct

SEQ ID NO: 17 - DNA, 300 bp, Artificial Sequence
  PglpF_SD3 variant of PglpF E. Coli promoter sequence of glpFKX operon
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagaacaaaacagct

SEQ ID NO: 18 - DNA, 300 bp, Artificial Sequence
  PglpF_SD4, variant of PglpF E. Coli promoter sequence of glpFKX operon
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaactaggaaacagct

SEQ ID NO: 19 - DNA, 300 bp, Artificial Sequence
  PglpF_SD5, variant of PglpF E. Coli promoter sequence of glpFKX operon
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaccgagaaacagct

SEQ ID NO: 20 - DNA, 300 bp, Artificial Sequence
  PglpF_SD6, variant of PglpF E. Coli promoter sequence of glpFKX operon
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagctaaacagct

SEQ ID NO: 21 - DNA, 300 bp, Artificial Sequence
  PglpF_SD7, variant of PglpF E. Coli promoter sequence of glpFKX operon
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagcaaaacagct

SEQ ID NO: 22 - DNA, 300 bp, Artificial Sequence
  PglpF_SD8, variant of PglpF E. Coli promoter sequence of glpFKX operon
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagaaaaacagct

SEQ ID NO: 23 - DNA, 300 bp, Artificial Sequence
  PglpF_SD9, variant of PglpF E. Coli promoter sequence of glpFKX operon
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaaggaaaaacagct

SEQ ID NO: 24 - DNA, 300 bp, Artificial Sequence
  PglpF_B29, variant of PglpF E. Coli promoter sequence of glpFKX operon
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgatttaattacagcatgcctacaagcatcgtggaggtccgtgactttcacgcatacaacaaacattaaccaaggaggaaacagct

SEQ ID NO: 25 - DNA, 300 bp, Artificial Sequence
  PglpF_B29, variant of PglpF E. Coli promoter sequence of glpFKX operon
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgatcagaatacagcatgcctacaagcatcgtggaggtccgtgactttcacgcatacaacaaacattaaccaaggaggaaacagct

SEQ ID NO: 26 - DNA, 107 bp, Artificial Sequence
  Plac_16UTR, variant of E. coli lac operon promoter
tgtgagttagctcactcattaggcaccccaggctttacactttatgcttccggctcgtatgttgtgtggaattgtgagcggataacaatttcaaggaggaaacagct

SEQ ID NO: 27 - DNA, 107 bp, Escherichia coli
  misc_feature (1)..(107): Plac, lac operon promoter
tgtgagttagctcactcattaggcaccccaggctttacactttatgcttccggctcgtatgttgtgtggaattgtgagcggataacaatttcacacaggaaacagct

SEQ ID NO: 28 - protein, 387 aa, Rouxiella badensis
  MISC_FEATURE (1)..(387): Bad MFS transporter, GeneBank ID WP_017489914.1
MSSRRLSIIFATFLLVSFLTGIAGALQAPTLSLFLTNEVKVRPLWVGLFYTVNALGGIVISFLLANYSDKKGDRRKLLFFCTLMAIGNSLIFAYSRDYLVLISVGVLLAAIGNASMPQLFALAREYADRSAHEVVMFSSMMRATLSLAWVLGPPISFTLALNYGFTLMYLCAAGVFIFSALMVWFFLPSVGRIEQPVDKVVVHVSAWKNRDVRLLFFASLLMWTCNIMYIIDMPLYITSDLGLPEGLAGLLMGAAAGLEIPVMLIAGYLVKRTGKRRLMLCAAVFGILFYLGLVLFQFKAALMILQLFNAIFIGIIAGIGMLYFQDLMPGRAGSATTLFTNSISTGAILAGVIQGTIVQNFGHYQVYWMALALAVGALVLMTRVKNV

SEQ ID NO: 29 - protein, 394 aa, Rosenbergiella nectarea
  MISC_FEATURE (1)..(394): Nec MFS transporter, GeneBank ID WP_092672081.1
MQSFTPPAPKGGNPVFMMFMLVTFFVSIAGALQAPTLSLYLSQELAAKPFMVGLFFTINAVTGIIISFILAKRSDRKGDRRRLLMFCCAMAIANALMFAFVRQYVVLITLGLILSALTSVVMPQLFALAREYADRTGREVVMFSSVMRTQMSLAWVIGPPISFALALNYGFITLYLVAAALFLLSLILIKTTLPSVPRLYPAEDLAKSAASGWKRTDVRFLFAASVLMWVCNLMYIIDMPLYISKSLGMPESFAGVLMGTAAGLEIPVMLLAGYLAKRVGKRPLVIVAAVCGLAFYPAMLVFHQQTGLLIIQLLNAVFIGIVAGLVMLWFQDLMPGKAGAATTLFTNSVSTGMIFAGLCQGLLSDLLGHQAIYVLATVLMVIALLLLLRVKEQA

SEQ ID NO: 30 - protein, 393 aa, Yersinia bercovieri
  MISC_FEATURE (1)..(393): YberC MFS transporter, GeneBank ID EEQ08298.1
MKSALTFSRRINPVFLAFFVVAFLSGIAGALQAPTLSLFLSTEVKVRPLWVGLFYTVNAIAGITVSFILAKRSDSRGDRRKLIMVCYLMAVGNCLLFAFNRDYLTLITAGVLLASVANTAMPQIFALAREYADSSAREVVMFSSIMRAQLSLAWVIGPPLSFMLALNYGFTLMFSIAAGIFVLSALVVWFILPSVPRAEPVVDAPVVVQGSLFADKNVLLLFIASMLMWTCNTMYIIDMPLYITASLGLPERLAGLLMGTAAGLEIPIMLLAGYSVRYFGKRKIMLFAVLAGVLFYTGLVLFKFKTALMLLQIFNAIFIGIVAGIGMLYFQDLMPGRAGAATTLFTNSISTGVILAGVLQGGLTETWGHDSVYVMAMVLSILALIICARVREA

SEQ ID NO: 31 - protein, 393 aa, Yersinia frederiksenii
  MISC_FEATURE (1)..(393): Fred MFS transporter, GeneBank ID WP_087817556.1
MKSALTFSRRINPVFLAFFVVAFLSGIAGALQAPTLSLFLSTEVKVRPLWVGLFYTVNAIAGITVSFVLAKRSDLRGDRRKLILVCYLMAVGNCLLFAFNRDYLTLITAGVLLAAVANTAMPQIFALAREYADNSAREVVMFSSIMRAQLSLAWVIGPPLSFMLALNYGFTLMFCIAAGIFVLSALVVWFILPSVQRAEPVMDAPTVAQGSLFADKDVLLLFIASMLMWTCNTMYIIDMPLYITASLGLPERLAGLLMGTAAGLEIPIMLLAGYSVRRFGKRKIMLFAVLAGVLFYTGLVLFKFKSALMLLQIFNAIFIGIVAGIGMLYFQDLMPGRAGAATTLFTNSISTGVILAGVLQGVLTETWGHNSVYVMAMILAILSLIICARVREA

SEQ ID NO: 32 - protein, 392 aa, Pantoea vagans
  MISC_FEATURE (1)..(392): Vag MFS transporter, GeneBank ID WP_048785139.1
MKSLLTRKRRINPVFLAFMAASFMIGVAGALQAPTLSLFLTREVQARPLWVGLFFTVNAIAGIVVSMLVAKRSDSRGDRRTLILFCCAMAFCNALLFAFTRHYLTLITLGVLLSALASVSMPQIFALAREYADQSAREAVMFSSVMRAQLSLAWVIGPPLSFALALNFGFVTLFLVAAALFLVCILLIKFTLPSVPRAEPLMRSGGMPLSGWRDRDVRLLFIASVTMWTCNTMYIIDMPLYISVTLGLPEKLAGLLMGTAAGLEIPVMLLAGHYAKRVGKRNLMLIAVAAGVLFYAGLAMFASQTALMALQLFNAVFIGIIAGIGMLWFQDLMPGRPGAATTMFTNSISTGMILAGVIQGTLSERFGHIAVYWLALGLAVAAFAMSARVKNV

SEQ ID NO: 33 - protein, 398 aa, Serratia marcescens
  MISC_FEATURE (1)..(398): Marc MFS transporter, GeneBank ID WP_060448169.1
MQRLSRLSLRINPIFAAFLLIAFLSGIAGALLTPTLSLFLTTEVKVRPLWVGLFYTANAVAGIVVSFLLAKRSDTRGDRRRLILLCCLMAVGNCLLFAFNRDYLTLITAGVLMSAVANTAMPQIFALAREYADSEAREVVMFSSVMRAQLSLAWVIGPPLSFALALNYGFTVMFLIAAVTFAVCVLLVGFMLPSVPRAAENEGLQGGVSAPIAPASAWRNRDVRLLFIASMLMWTCNTLYIIDMPLYITADLGLPEGLAGVLMGTAAGLEIPAMLLAGYYVKRFGKRNMMLLAVVAGVLFYLGLTVLESKPALIALQLLNAVFIGIVAGIGMLYFQDLMPGRPGAATTLFTNSISTGVILAGVLQGALVENLGHGSVYWMAALLALAALGMSAKVREV

SEQ ID NO: 34 - protein, 505 aa, Klebsiella pneumoniae
  MISC_FEATURE (1)..(505): ScrY, sucrose porin, GeneBank ID CAA40657.1
MYKKRKLAILIALLTGTAAAHGQTDLNSIEARLAALEKRLQDAETRASTAESRAASAEQKVQQLTQQQQQTQATTQQVARRTTQLEEKAERPGGFEFHGYARSGVIMNDSAASTKSGAYMTPAGETGGAIGRLGNQADTYVEMNLEHKQTLDNGATTRFKVMVADGQTTYNDWTASSSDLNVRQAFVELGNLPTFEGPFKGSTLWAGKRFDRDNFDIHWIDSDVVFLAGTGGGIYDVKWNDSLRSNFSLYGRNFGDIADSSNSVQNYIVSMNNFAGPVQMMVSGMRAKDNDDRQDANGNLVKGDAANTGVHALLGLHNESFYGLRDGTSKTALLYGHGLGAEVKGIGSDGALRPGANTWRFASYGTTPLSDRWFIAPAVLAQSSKDRYVDGDSYQWATLNLRLIQEVTQNFALAWEGSYQYMDLQPEGYNDRHAVNGSFYKLTFAPTFKVGSIGDFFSRPEIRFYTSWMDWSKKLDNYANDDALGSNGFKSGGEWSFGMQMETWF

SEQ ID NO: 35 - protein, 456 aa, Klebsiella pneumoniae
  MISC_FEATURE (1)..(456): ScrA, sucrose-specific enzyme II, GeneBank ID CAA40658.1
MDFEQISRSLLPLLGGKENIASAAHCATRLRLVLVDDALADQQAIGKIDGVKGCFRNAGQMQIIFGTGVVNKVYAAFIQAAGISESSKSEAADLAAKKLNPFQRIARLLSNIFVPIIPAIVASGLLMGLLGMVKTYGWVDPSNALYIMLDMCSSAAFIILPILIGFTAAREFGGNPYLGATLGGILTHPALTNAWGVAAGFHTMNFFGIEVAMIGYQGTVFPVLLAVWFMSMVEKRLRRVIPDALDLILTPFLTVIISGFIALLLIGPAGRALGDGISFILSTLISHAGWLAGLLFGGLYSVIVITGIHHSFHAIEAGLLGNPSIGVNFLLPIWAMANVAQGGACFAVWFKTKDAKIKAITLPSAFSAMLGITEAAIFGINLRFVKPFIAALVGGAAGGAWVVSMHVYMTAVGLTAIPGMAIVQASSLLNYIIGMAIAFAVAFALSLTLKYKTDAE

SEQ ID NO: 36 - protein, 466 aa, Salmonella enterica subsp. enterica serovar Typhimurium
  MISC_FEATURE (1)..(466): ScrB, beta-fructofuranosidase, GeneBank ID CAA47974.1
MSLPSRLPAILQAVMQGQPRALADSHYPRWHHAPVTGLMNDPNGFIEFAGRYHLFYQWNPLACDHTFKCWAHWSSIDLLHWQHEPIALMPDEEYDRNGCYSGSAVDNNGTLTLCYTGNVKFAEGGRTAWQCLATENADGTFRKIGPVLPLPEGYTGHVRDPKVWRHEDLWYMVLGAQDRQKRGKVLLFSSADLHQWTSMGEIAGHGINGLDDVGYMWECPDLFPLGDQHILICCPQGIAREEECYLNTYPAVWMAGEFDYAAGAFRHGELHELDAGFEFYAPQTMLTSDGRRLLVGWMGVPEGEEMLQPTLNNGWIHQMTCLRELEFINGQLYQRPLRELSALRGEANGWSGNALPLAPMEIDLQTRGGDMLSLDFGGVLTLECDASGLRLARRSLASDEMHYRYWRGNVRSLRVFIDQSSVEIFINGGEGVMSSRYFPACSGQLTFSGITPDAFCYWPLRTCMVE

SEQ ID NO: 37 - protein, 334 aa, Salmonella enterica subsp. enterica serovar Typhimurium
  MISC_FEATURE (1)..(334): ScrR, sucrose repressor, GeneBank ID CAA47975.1
MKTKRVTIKDIAEQAGVSKATASLVLNGRGKELRVAQETRERVLSIARKHHYQPSIHARSLRNNRSHTIGLVVPEITNHGFAVFAHELEMLCREAGVQLLISCTDENPGQESVVVNNMIARQVDGMIVASCMHNDADYLKLSQQLPVVLFDRCPNESALPLVMTDSITPTAELISRIAPQHSDEFWFLGGQARLSPSRDRLTGFTQGLAQAGIALRPEWVINGNYHPSSGYEMFAALCARLGRPPKALFTAACGLLEGVLRYMSQHHLLDSDIHLTSFDDHYLYDSLSLRIDTVQQDNRQLAWHCYDLISQLIEGDTPETLQRYLPATLQFRHQ

SEQ ID NO: 38 - protein, 483 aa, Avibacterium gallinarum
  MISC_FEATURE (1)..(483): SacC_AgaI, glycoside hydrolase family 32 protein, GeneBank ID WP_103853210.1
MIIFNEGKYKSLYAAEQGELEKIAQTVAQDQDFRPVYHLAPPTGLLNDPNGLIFDGEKYHLFYQWYPFDALHGMKHWQHFITQDFKQFSQADLLVPCELYESHGCYSGGAVKIGDQIAVFYTGNTRRPSDNQRVPYQNLAIFSKDGKLLSKRPLIEQAPQGYTEHVRDPKPFLTKDGKIRFICGAQRENLTGTALVFEMDNLADTPRLLGELALPAFDNQGVFMWECPDLSQMGDKSLFIWSPQGKARELEQYQNNYHAVYALGELADRQFHAEQIAELDQGFDFYAPQTFSGTQTMLLGWVGLPDLSYPTDLYKWHSMLSMPRQLRLQDGKIYQQPIENIYKNLTALQSITVEKEAEIADLDRAYLKFDANAQPFSLKFFNNAQNQRLILSYDGEMLCLDRSQTEQTDSMKSFGDKRYCRIEDLRQVEIFFDRSVAEIFLNQGEKAMTSRFFICARENQLCTDKPLTLQVGYPKKIEVDYTK

SEQ ID NO: 39 - protein, 548 aa, Arthrobacter globiformis
  MISC_FEATURE (1)..(548): Bff, beta-fructofuranosidase protein, GeneBank ID BAD18121.1
MERTCITVRAIVRFHIEQRQTIVNKQRTKRGILTAALSIGALGATLISGPAVAATDAAPGFPQPTEHTQKAYSPTDNFTSRWTRADAKQLKAMSDPDAGSRENSMPTEYTMPTVSQDFPDMSNEKVWVWDTWPLIDENANQYSVNGQEIIFSLVADRKLGFDERHQYARIGYFYRPAGIPADERPEDGGWTYGGQVFDEGVTGKIFEDQSFTHQTQWSGSARVSKNGEIKLFFTDVAFYRDKDGQDVKPYDSRIALSVGHVHSNKKGVKLTGFNKVKELLQADGKNYQNAAQNSYYNFRDPFTFVDPAHPGETYMVFEGNSAMDRDEAKCTAEDLGYREGETNGETVEQVNNSGATYQIGNVGLARAKNKALTEWEFLPPILSANCVTDQTERPQIYMQDGKYYLFTISHRSTFATGIDGPEGVYGFVGNGIRSDYQPLNRGSGLALGSPTNLNFAAGTPFAPDYNQHPGQFQAYSHYVMPGGLVQSFIDTIGTKDNFVRGGTLGPTVKLNIKGDSATVDYNYGDNGLGGWADIPANRELKNSKAVAK

SEQ ID NO: 40 - DNA, 999 bp, Artificial Sequence
  lgta coding nucleotide sequence
atgcaaccgctggtctccgtgctgatctgtgcttacaatgtggaaaaatacttcgcccaatcgctggccgcagtcgtgaatcaaacgtggcgcaacctggaaattctgatcgtggatgacggcagtaccgatggtacgctggcgatcgccaaagattttcagaaacgtgactcccgcattaaaatcctggcacaggctcaaaacagtggcctgattccgtccctgaatatcggtctggatgaactggcgaaaagtggcatgggtgaatatatcgcacgcaccgatgctgatgacattgcggccccggactggattgaaaaaatcgtcggcgaaatggaaaaagatcgtagcattatcgcgatgggtgcctggctggaagtgctgtctgaagaaaaagatggcaatcgtctggcacgccatcaccgtcatggtaaaatctggaaaaaaccgacgcgtccggaagatattgccgactttttcccgtttggcaacccgattcacaacaataccatgatcatgcgtcgctcagttattgatggcggtctgcgctataatacggaacgtgattgggcggaagactatcagttctggtacgatgtctcgaaactgggtcgcctggcgtattacccggaagccctggtgaaatatcgtctgcatgccaaccaagttagctctaaatactctatccgccaacacgaaattgcacagggcatccaaaaaaccgctcgtaatgattttctgcagtcaatgggttttaaaacgcgcttcgactcgctggaatatcgtcaaattaaagcggttgcctacgaactgctggaaaaacatctgccggaagaagattttgaacgcgcgcgtcgctttctgtatcagtgcttcaaacgtaccgacacgctgccggcaggtgcttggctggatttcgcagctgacggtcgcatgcgtcgcctgtttaccctgcgtcaatacttcggcattctgcaccgcctgctgaaaaaccgttaa

SEQ ID NO: 41 - DNA, 1005 bp, Artificial Sequence
  pmnagT coding nucleotide sequence
atggaaaataaaccgctggttagcgttctgatttgcgcctataatgtggaaaaatacatcgaagaatgcatcaacgccgttattaaccagacctataaaaacctggaaatcatcattgtgaatgatggcagcagcgataacacctattttctgctgaaaaaactggccgaaaaagacaaccgtatcaagatcctgaacttcaacaaccatattggcattattagcgcactgaatgaaggcctgaaagaaattgccggtgaatatattgcacgtaccgattcagatgatatcaccaaaccggattggatcgaaaaaattctgacctgtatgcagaacgacccgaaaattatcgcaatgggtagctatctgaccgttctgagcgaagaaaataatggtagcgtgctggccaatcaccataaaaacaaagtggaatggaaaaacccgctggaacataaagatatcgtggaaaaaatgctgtttggcaacccgattcataataacagcatggttatgcgcagcgagatctataccaaatatcacctgatttatgatccggattatcattatgccgaggactataaattctggctggaagttagccgtattggtaaactggcaaattatccggaaagcctggtttattatcgtctgcatcgtaatcagaccagcagcattcataattcccagcaagaaatcaacggtaaaaaactgcgtctgcaggcactgaactattatctgaaagatctgggcattgattatcagctgccggaaaaatttctgttcaaagatattgcactgctgcaagagatcttttatgaacgtggtatgttccgcgaaaacattattcgtcgcattatctatgagtgctatctgagcctgggcgagtataattacaaagatatctactacttcctgatcaacaaaaacaactttctgagcatcaaagacaaattcaaaatcatcaaaaaatacctgcgtccggacaaatatagcagcacctattaa

SEQ ID NO: 42 - DNA, 990 bp, Artificial Sequence
  HD0466 coding nucleotide sequence
atgaccacactggttagcgttctgatttgtgcctataacgtggaaaaatacatcgatgaatgtctgaatgcagttattgcccagacctataaaaacctggaaattatcgttgtgaatgatggtagcaccgatggcaccctggcaaaactgcgtcagtttgaagcaaaagatccgcgtgttaaaatcatcgataacattgttaatcagggcaccagcaaaagcctgaatattggtattcagtattgtcagggcgaaattattgcacgtaccgattcagatgatatcgtggatattcattggatcgaaaccctgatgcgtgaactggataatagtccggaaaccattgcaattagcgcctatctggaatttctggccgaaaaaggtaatggtagcaaactgagccgtagccgtaaacatggtaaaaatgcagaaaatccgattagcagcgaagcaattagccagcgtatgctgtttggtaatccggttcataacaatgtggcactggttcgtcgtaaagtgtttagcgaatatggtctgcgttttgatccggattatattcatgccgaggattacaaattttggttcgaagtgagcaaactgggtaaaatgcgtacctatccgaaagcgctggttaaatatcgtctgcatgcaacccaggttagcagcgcatataatcagaaacagcgtagcattgccaaaaaaatcaaacgtgaagccatcagccattatctgcagcagtatggcattcagctgccggaaaaactgaccattcatgacctgtttagcatttttagtccgcagattgaactgagcctgaccgttgcaaataaacaagaactgttttggagcctggcaaccagcctgagcgaatatcattttcgtgatctgctgaaaatctacagcctggatatttttcatcagctgagcttcaaatacaaaaagcgcatctttcgcaaatttctgctgccgaatcgttatccgagcgttatt

SEQ ID NO: 43 - DNA, 1320 bp, Artificial Sequence
  GalTK coding nucleotide
atgatctctgtctacatcatcagtctgaaagaatcgcagcgtcgtctggatacggaaaaactggttctggaatcgaacgaaaaatttaaaggccgttgtgtgtttcagattttcgatgcgatctctccgaaacatgaagacttcgaaaaattcgttcaagaactgtacgatagctctagtctgctgaaatcggattggttccatagcgactattgctaccaggaactgctgccgcaagaatttggttgttatctgagccactacctgctgtggaaagaatgcgttaaactgaatcagccggtggttattctggaagatgacgtcgcgctggaatctaactttatgcaggccctggaagattgtctgaaaagtccgtttgacttcgtccgtctgtatggccattactggggcggtcacaaaaccaatctgtgcgcgctgccggtttataccgaaacggaagaagcggaagcctccattgaaaaaaccccgatcgaaaattatgaagtgaccagcccgccgccgccgaacccgacccgcgatacgcagcaagacttcatcaccgaaacgcagcaagatccgaaagaactgtcggaaccgtgcaaaattgccccgcagaaaatcagcttcaaccaagtcgtgttcaagaaaattaaacgtaaactgaaccgcttcatcggtagcatcctggcgcgtaccgaagtctataaaaatatcgtggccaaatacgatgacctgaccacgaaatatgacgatctgaccacgaaatatgatgatctgacgaccaaatatgacgacctgacgacgaaatacgatgacctgaacaaaaacatcgcagaaaaatacgatgaactgatgggcaaatacgaatcgctgctggctaaagaagtgaacatcaaagaaaccttctgggaatcccgtgcggattcagaaaaagaagccctgtttctggaccatttctatctgaccagcgtttacgtcgcaaccacggctggctattacctgaccccgaaaggtgcaaaaaccttcattgaagctacggaacgctttaaaattatcgaaccggttgatatgttcattaacaatccgacctatcatgatattgccaactttacgtacgtgccgtgtccggtttccctgaacaaacacgcattcaactcaaccatccagaacgctaaaaaaccggatattagcctgaaaccgccgaaaaaatcttacttcgataacctgttttatcacaaatttaacgcacgcaaatgcctgaaagcattcaataaatacagtaaacagtacgccccgctgaaaaccccgaaagaagtctaa

SEQ ID NO: 44 - DNA, 789 bp, Artificial Sequence
  cvb3galT coding nucelotide sequence
atggacaccatcatgattaaacgtccgctggttagcgttattctgccggtgaataaaaacaatccgcatctggaagaagcaatccagagcattaaaaaccagacctataaagagctggaactgatcattattgccaacaactgcgaggataacttttatagcctgctgctgaaatatcaggaccagaaaaccaaaattatccgcaccagcatcaaatatctgccgtttagcctgaatctgggtgttcatctgagccagggtgaatatattgcacgtatggattcagatgatatcagcgttctggatcgcattgaaaaacaggttaaacgctttctgaatacaccggaactgagcattctgggtagcaatgttgaatatatcaatgaagccagcgaaagcattggctatagcaactatccgctggatcatagcagcattgttaatagctttccgtttcgttgtaatctggcacatccgaccattatggttaaaaaagaagtgattaccacgcttggtggctatatgtatggtagcctgagcgaagattatgatctgtggattcgtgcaagccgtcatggcaatttcaaatttagcaatattgatgaaccgctgctgaagtaccgtattcataaaggtcaggcaaccaataaaagcaacgcctataacatctttgcctttgatagcagcctgaaaatccgtgaatttctgctgaatggtaatgtgcagtatctgctgggtgcagcacgtggtttttttgcatttctgtatgtgcgcttcatcaaaaaatga

SEQ ID NO: 45 - DNA, 909 bp, Artificial Sequence
  futC coding nuelcotide sequence
atggcgttcaaagtggtccaaatctgcggtggtctgggtaatcaaatgttccaatatgccttcgctaaatcgctgcaaaaacacagtaataccccggtcctgctggatattacgagttttgattggtccgaccgtaaaatgcagctggaactgttcccgattgatctgccgtatgcgagcgccaaagaaatcgcaattgctaaaatgcagcatctgccgaaactggttcgtgatgcgctgaaatgcatgggctttgaccgcgtcagtcaagaaatcgtgttcgaatatgaaccgaaactgctgaaaccgtcccgtctgacctatttctttggttactttcaggacccgcgttacttcgacgccatctctccgctgattaaacaaacctttacgctgccgccgccgccggaaaacaacaaaaacaacaacaaaaaagaagaagaatatcagtgcaaactgagcctgatcctggcggccaaaaactctgtgtttgttcacattcgtcgcggcgattacgtgggcatcggttgtcagctgggtattgactatcagaaaaaagcgctggaatacatggccaaacgtgttccgaatatggaactgtttgtcttctgcgaagatctggaatttacccaaaacctggacctgggctatccgttcatggatatgaccacgcgcgacaaagaagaagaagcgtattgggatatgctgctgatgcagagctgtcaacatggtattatcgctaatagcacgtattcttggtgggcagcttacctgattgaaaacccggaaaaaattatcattggcccgaaacattggctgtttggtcacgaaaatatcctgtgtaaagaatgggtgaaaatcgaatcacacttcgaagttaaatcgcagaaatataacgcgctgggctaa

SEQ ID NO: 46 - DNA, 879 bp, Artificial Sequence
  mtun coding nucleotide sequence
atggtgattacccatctgattggtggtctgggtaaccagatgtttcagtatgcagcaggtcgtgcagttagcctggaacgtggtgttagcctgagcctggatattagcggttttgcaaattatggtctgcatcagggttttgaactgcagcgtatctttaattgtaccgcagaaattgcaaatgaagccgatgttcgtggtattttaggttggcagagcagtccgcgtattcgtcagctgctgagccgtcagaatatggcaatttttcgtcgtgaaggttttgttgtggaaccgcattttcattattggcagggtattaaaagcgttccgcgtgattgttatctgaccggctattggcagagtgaacagtattttctggaagcagcagcacagattcgtgcagattttacctttaaactgccgctggataaccagaatattgaactggccaaacaaatcaatgccgttaatgcggttagcctgcatgttcgtcgtggtgattatgcaaatacaccggaaaccaccgcaacacatggtctgtgtagtctggattattatcgtgttgccattcgtcatattgcagaacaggttcagcagccgcatttttttgtttttagtgatgatattgcctgggtgaagaacaacctgagtattgattttccgtgccagtatgtggatcataatcagggtgcagaaagctataatgatatgcgtctgatgagcatgtgccgtcatcatattattgcaaacagcagctttagttggtggggtgcatggctgaatccgaatgttaacaaaattgttgttgcaccgagccgttggtttgccaaacagaccgatgtgcgtgatctgctgccgcaaggttggattaaacagtaa

SEQ ID NO: 47 - DNA, 879 bp, Artificial Sequence
  smob coding nucleotide sequence
atgatcatcagccagattattggtggtctgggtaatcagatgtttcagtatgcagcaggtcgtgcactgagcctggttcgtggtcagccgctgctgctggatgttaccggttttgcaggttatggtctgcatcagggttttgaactgcagcgtgtttttgattgtccgattggtattgcaaccgaagaagatgttcgcggtattttaggttggcagtttagcgcaggtattcgtcgtattgttgcacgtcctggtatggcagcatttcgtcgtaaaggttttattgtggaaccgcactttcattattggcctgagattaaaaacgttccgcgtgattgttatctgcttggttattggcagagcgaacgttattttcgtgcagcaaccgcagatattcgtgcagatttttcatttaaaagtccgctggttaatcgcaatgccgaaaccgcagcacagattgatcaggttaatgcaattagcctgcatatgcgtcgtggtgattatgtgaataatccgaaaaccagcgcaacccatggtctgtgtagcctggattattatcaggcagcaatcaaatttgttagcgaacgtgttgaagaaccgtttttctttatcttctccgatgatattgcatgggtgaaagcaaatctgaaactggattttccgtgccagtatgtggatcataatcatggtgcagaaagcttcaatgatatgcatctgatgagcctgtgtcagcatcatattattgcaaacagcagctttagttggtggggtgcatggctgaatagcgatccgaaaaaaatcgttctggcaccgaaaaaatggttcgccaacaaaaacaacatcaaagacctgtttccgcctggttgggttagcctgtaa

SEQ ID NO: 48 - DNA, 730 bp, Artificial Sequence
  DNA-binding transcriptional repressor GlpR
atgaaacaaacacaacgtcacaacggtattatcgaactggttaaacagcagggttatgtcagtaccgaagagctggtagagcatttctccgtcagcccgcagactattcgccgcgacctcaatgagctggcggagcaaaacctgatcctggccatcatggcggtgcggcgctgccttccagttcggttaacacgccgtggcacgatcgcaaggccacccagaccgaagaaaaagagcgcatcgcccgcaaagtggcggagcaaatccccaatggctcgacgctgtttatcgatatcggcaccacgccggaagcggtagcgcacgcactgctcaatcacagcaatttgcgcattgtcaccaacaatctcaacgttgctaacacgttgatggtaaaagaagattttcgcatcattctcgccggtggcgaattacgcagccgcgatggcgggatcattggcgaagcgacgctcgattttatctcccagttccgccttgatttcggcattctggggataagcggcatcgatagcgacggctcgctgctggagttcgattaccacgaagttcgcaccaaacgcgccattattgagaactcgcgccacgttatgctggttgtcgatcactcgaaatttggccgtaacgcgatggtcaatatgggcagcatcagcatggtagatgccgtctacaccgacgccccgccgccagtaagcgtgatgcaggtgctgacgga

SEQ ID NO: 49 - protein, 292 aa, Sideroxydans lithotrophicus
  MISC_FEATURE (1)..(292): fucT54 -1,2-fucosyltransferas
MVISNIIGGLGNQMFQYAAARALSLKLEVPLKLDISGFTNYALHQGFELDRIFGCKIEIASEADVHEILGWQSASGIRRVVSRPGMSIFRRKGFVVEPHFSYWNGIRKITGDCYLAGYWQSEKYFLDAAVEIRKDFSFKLPLDSHNAELAEKIDQENAVSLHIRRGDYANNPLTAATHGLCSLDYYRKSIKHIAGQVRNPYFFVFSDDIAWVKDNLEIEFPSQYVDYNHGSMSFNDMRLMSLCKHHIIANSSFSWWGAWLNPNPEKVVIAPERWFANRTDVQDLLPPGWVKL

SEQ ID NO: 50 - DNA, 24 bp, Artificial Sequence
  Oligo O48, galK.for
  misc_feature: Oligo O48, galK.for
cccagcgagacctgaccgcagaac

SEQ ID NO: 51 - DNA, 24 bp, Artificial Sequence
  Oligo O49, galK.rev
  misc_feature (1)..(24): Oligo O49, galK.rev
ccccagtccatcagcgtgactacc

SEQ ID NO: 52 - DNA, 6706 bp, Escherichia coli
  misc_feature: CA gene cluster
atgtcaaaagtcgctctcatcaccggtgtaaccggacaagacggttcttacctggcagagtttctgctggaaaaaggttacgaggtgcatggtattaagcgtcgcgcatcgtcattcaacaccgagcgcgtggatcacatttatcaggatccgcacacctgcaacccgaaattccatctgcattatggcgacctgagtgatacctctaacctgacgcgcattttgcgtgaagtacagccggatgaagtgtacaacctgggcgcaatgagccacgttgcggtctcttttgagtcaccagaatataccgctgacgtcgacgcgatgggtacgctgcgcctgctggaggcgatccgcttcctcggtctggaaaagaaaactcgtttctatcaggcttccacctctgaactgtatggtctggtgcaggaaattccgcagaaagagaccacgccgttctacccgcgatctccgtatgcggtcgccaaactgtacgcctactggatcaccgttaactaccgtgaatcctacggcatgtacgcctgtaacggaattctcttcaaccatgaatccccgcgccgcggcgaaaccttcgttacccgcaaaatcacccgcgcaatcgccaacatcgcccaggggctggagtcgtgcctgtacctcggcaatatggattccctgcgtgactggggccacgccaaagactacgtaaaaatgcagtggatgatgctgcagcaggaacagccggaagatttcgttatcgcgaccggcgttcagtactccgtgcgtcagttcgtggaaatggcggcagcacagctgggcatcaaactgcgctttgaaggcacgggcgttgaagagaagggcattgtggtttccgtcaccgggcatgacgcgccgggcgttaaaccgggtgatgtgattatcgctgttgacccgcgttacttccgtccggctgaagttgaaacgctgctcggcgacccgaccaaagcgcacgaaaaactgggctggaaaccggaaatcaccctcagagagatggtgtctgaaatggtggctaatgacctcgaagcggcgaaaaaacactctctgctgaaatctcacggctacgacgtggcgatcgcgctggagtcataagcatgagtaaacaacgagtttttattgctggtcatcgcgggatggtcggttccgccatcaggcggcagctcgaacagcgcggtgatgtggaactggtattacgcacccgcgacgagctgaacctgctggacagccgcgccgtgcatgatttctttgccagcgaacgtattgaccaggtctatctggcggcggcgaaagtgggcggcattgttgccaacaacacctatccggcggatttcatctaccagaacatgatgattgagagcaacatcattcacgccgcgcatcagaacgacgtgaacaaactgctgtttctcggatcgtcctgcatctacccgaaactggcaaaacagccgatggcagaaagcgagttgttgcagggcacgctggagccgactaacgagccttatgctattgccaaaatcgccgggatcaaactgtgcgaatcatacaaccgccagtacggacgcgattaccgctcagtcatgccgaccaacctgtacgggccacacgacaacttccacccgagtaattcgcatgtgatcccagcattgctgcgtcgcttccacgaggcgacggcacagaatgcgccggacgtggtggtatggggcagcggtacaccgatgcgcgaatttctgcacgtcgatgatatggcggcggcgagcattcatgtcatggagctggcgcatgaagtctggctggagaacacccagccgatgttgtcgcacattaacgtcggcacgggcgttgactgcactatccgcgagctggcgcaaaccatcgccaaagtggtgggttacaaaggccgggtggtttttgatgccagcaaaccggatggcacgccgcgcaaactgctggatgtgacgcgcctgcatcagcttggctggtatcacgaaatctcactggaagcggggcttgccagcacttaccagtggttccttgagaatcaagaccgctttcgggggtaatgatgtttttacgtcaggaagactttgccacggtagtgcgctccactccgcttgtctctctcgactttattgtcgagaacagtcgcggcgagtttctgcttggcaaaagaaccaaccgcccggcgcagggttactggtttgtgccgggagggcgcgtgcagaaagacgaaacgctggaagccgcatttgagcggctgacgatggcggaactggggctgcgtttgccgataacagcaggccagttttacggtgtctggcagcacttttatgacgataacttctctggcacggatttcaccactcactatgtggtgctcggttttcgcttcagagtatcggaagaagagctgttactgccggatgagcagcatgacgattaccgctggctgacgtcggacgcgctgctcgccagtgataatgttcatgctaacagccgcgcctattttctcgctgagaagcgtaccggagtacccggattatgaaaatactggtctacggcattaactactcgccggagttaaccggcatcggcaaatacaccggcgagatggtggaatggctggcggcacaaggtcatgaggtgcgggtcattaccgcaccgccttactacccgcaatggcaggtgggcgagaactattccgcctggcgctacaaacgagaagagggggccgccacggtgtggcgctgcccgctgtatgtgccaaaacagccgagcaccctgaaacgcctgttgcatctgggcagttttgccgtcagcagtttctttccgctgatggcgcaacgtcgctggaagccggatcgcattattggcgtggtgccaacgctgttttgcgcgccgggaatgcgcctgctggcgaaactctctggtgcgcgtaccgtgctgcatattcaggattacgaagtggacgccatgctggggctgggccttgccggaaaaggcaaaggcggcaaagtggcacagctggcaacggcgttcgaacgtagcggactgcataacgtcgataacgtctccacgatttcgcgttcgatgatgaataaagccatcgaaaaaggcgtggcggcggaaaacgtcatcttcttccccaactggtcggaaattgcccgttttcagcatgttgcagatgccgatgttgatgcccttcgtaaccagcttgacctgccggataacaaaaaaatcattctttactccggcaatattggtgaaaagcaggggctggaaaacgttattgaagctgccgatcgtctgcgcgatgaaccgctgatttttgccattgtcgggcagggcggcggcaaagcgcggctggaaaaaatggcgcagcagcgtggactgcgcaacatgcaatttttcccgctgcaatcgtatgacgctttacccgcactgctgaagatgggcgattgccatctggtggtgcaaaaacgcggcgcggcagatgccgtattgccgtcgaaactgaccaatattctggcagtaggcggtaacgcggtgattactgctgaagcctacacagaactggggcagctttgcgaaacctttccgggcattgcggtttgcgttgaaccggaatcggtcgaggcgctggtggcggggatccgtcaggcgctcctgctgcccaaacacaacacggtggcacgtgaatatgccgaacgcacgctcgataaagagaacgtgttacgtcaatttataaatgatattcggggataattatggcgcagtcgaaactctatccagttgtgatggcaggtggctccggtagccgcttatggccgctttcccgcgtactttatcccaagcagtttttatgcctgaaaggcgatctcaccatgctgcaaaccaccatctgccgcctgaacggcgtggagtgcgaaagcccggtggtgatttgcaatgagcagcaccgctttattgtcgcggaacagctgcgtcaactgaacaaacttaccgagaacattattctcgaaccggcagggcgaaacacggcacctgccattgcgctggcggcgctggcggcaaaacgtcatagcccggagagcgacccgttaatgctggtattggcggcggatcatgtgattgccgatgaagacgcgttccgtgccgccgtgcgtaatgccatgccatatgccgaagcgggcaagctggtgaccttcggcattgtgccggatctaccagaaaccggttatggctatattcgtcgcggtgaagtgtctgcgggtgagcaggatatggtggcctttgaagtggcgcagtttgtcgaaaaaccgaatctggaaaccgctcaggcctatgtggcaagcggcgaatattactggaacagcggtatgttcctgttccgcgccggacgctatctcgaagaactgaaaaaatatcgcccggatatcctcgatgcctgtgaaaaagcgatgagcgccgtcgatccggatctcaattttattcgcgtggatgaagaagcgtttctcgcctgcccggaagagtcggtggattacgcggtcatggaacgtacggcagatgctgttgtggtgccgatggatgcgggctggagcgatgttggctcctggtcttcattatgggagatcagcgcccacaccgccgagggcaacgtttgccacggcgatgtgattaatcacaaaactgaaaacagctatgtgtatgctgaatctggcctggtcaccaccgtcggggtgaaagatctggtagtggtgcagaccaaagatgcggtgctgattgccgaccgtaacgcggtacaggatgtgaaaaaagtggtcgagcagatcaaagccgatggtcgccatgagcatcgggtgcatcgcgaagtgtatcgtccgtggggcaaatatgactctatcgacgcgggcgaccgctaccaggtgaaacgcatcaccgtgaaaccgggcgagggcttgtcggtacagatgcaccatcaccgcgcggaacactgggtggttgtcgcgggaacggcaaaagtcaccattgatggtgatatcaaactgcttggtgaaaacgagtccatttatattccgctgggggcgacgcattgcctggaaaacccggggaaaattccgctcgatttaattgaagtgcgctccggctcttatctcgaagaggatgatgtggtgcgtttcgcggatcgctacggacgggtgtaaacgtcgcatcaggcaatgaatgcgaaaccgcggtgtaaataacgacaaaaataaaattggccgcttcggtcagggccaactattgcctgaaaaagggtaacgatatgaaaaaattaacctgctttaaagcctatgatattcgcgggaaattaggcgaagaactgaatgaagatatcgcctggcgcattggtcgcgcctatggcgaatttctcaaaccgaaaaccattgtgttaggcggtgatgtccgcctcaccagcgaaaccttaaaactggcgctggcgaaaggtttacaggatgcgggcgttgacgtgctggatattggtatgtccggcaccgaagagatctatttcgccacgttccatctcggcgtggatggcggcattgaagttaccgccagccataatccgatggattataacggcatgaagctggttcgcgagggggctcgcccgatcagcggagataccggactgcgcgacgtccagcgtctggctgaagccaacgactttcctcccgtcgatgaaaccaaacgcggtcgctatcagcaaatcaacctgcgtgacgcttacgttgatcacctgttcggttatatcaatgtcaaaaacctcacgccgctcaagctggtgatcaactccgggaacggcgcagcgggtccggtggtggacgccattgaagcccgctttaaagccctcggcgcgcccgtggaattaatcaaagtgcacaacacgccggacggcaatttccccaacggtattcctaacccactactgccggaatgccgcgacgacacccgcaatgcggtcatcaaacacggcgcggatatgggcattgcttttgatggcgattttgaccgctgtttcctgtttgacgaaaaagggcagtttattgagggctactacattgtcggcctgttggcagaagcattcctcgaaaaaaatcccggcgcgaagatcatccacgatccacgtctctcctggaacaccgttgatgtggtgactgccgcaggtggcacgccggtaatgtcgaaaaccggacacgcctttattaaagaacgtatgcgcaaggaagacgccatctatggtggcgaaatgagcgcccaccattacttccgtgatttcgcttactgcgacagcggcatgatcccgtggctgctggtcgccgaactggtgtgcctgaaagataaaacgctgggcgaactggtacgcgaccggatggcggcgtttccggcaagcggtgagatcaacagcaaactggcgcaacccgttgaggcgattaaccgcgtggaacagcattttagccgtgaggcgctggcggtggatcgcaccgatggcatcagcatgacctttgccgactggcgctttaacctgcgcacctccaataccgaaccggtggtgcgcctgaatgtggaatcgcgcggtgatgtgccgctgatggaagcgcgaacgcgaactctgctgacgttgctgaacgagtaa

(19) United States
(12) Patent Application Publication
(10) Pub. No.: US 2024/0327885 A1
(43) Pub. Date: Oct. 3, 2024

(54) METHODS OF PRODUCTING HMO BLEND PROFILES WITH LNFP-1 AND 2'-FL AS THE PREDOMINANT COMPOUNDS

(71) Applicant: DSM IP Assets B.V., Heerlen (DK)

(72) Inventors: Manos PAPADAKIS, Hørsholm (DK); Ted JOHANSON, Hørsholm (DK); Peter BECKER, Hørsholm (DK)

(21) Appl. No.: 18/561,123

(22) PCT Filed: May 17, 2022
(86) PCT No.: PCT/EP2022/063317

§ 371 Date: Nov. 15, 2023

(30) Foreign Application Priority Data
May 17, 2021 (DK) PA 2021 70247
Jul. 20, 2021 (DK) PA 2021 70390

(51) Int. Cl. C12P 19/00; C12N 9/10

(52) U.S. Cl. CPC C12P 19/00; C12N 9/1051; C12Y 204/01086

(57) ABSTRACT

The present disclosure relates to a method for the production of a human milk oligosaccharide (HMO) blend with LNFP-I and 2′-FL as the predominant HMO's, the method comprising the steps of providing a genetically engineered cell, which comprises a heterologous β-1,3-N-acetyl-glucosaminyltransferase protein, a heterologous β-1,3-galactosyltransferase protein, a heterologous α-1,2-fucosyltransferase protein, and expresses functionally the colanic acid gene cluster, comprises a native or heterologous regulatory or episomal element for controlling the expression of the proteins and optionally express a heterologous sugar transporter, and culturing the cell in a suitable cell culture medium to express said proteins and to produce an HMO blend.

### CROSS-REFERENCE TO RELATED APPLICATIONS

This application is a national stage entry pursuant to 35 U.S.C. § 371 of International Application No. PCT/EP2022/063317, filed on May 17, 2022, which claims priority to Denmark Application No. PA 2021 70390, filed on Jul. 20, 2021 and Denmark Application No. PA 2021 70247, filed on May 17, 2021, the entire contents of all of which are hereby incorporated by reference in their entirety.

### SEQUENCE LISTING

This instant application contains a Sequence Listing which has been submitted in a ASCII text file via Patent Center and is hereby incorporated by reference in its entirety. Said text file, created on Jun. 24, 2024, is named 032991-8007 Sequence Listing.txt, and is 104,817 bytes in size.

### FIELD

This disclosure relates to a method of producing mixtures of various human milk oligosaccharides (HMOs) with unique HMO blend profiles, consisting predominantly of LNFP-I and 2′-FL and of other HMOs in less significant amounts. The less abundant HMOs might be LNT, LNT-II or DFL. The strategies for achieving specific HMO blends include strain engineering and fermentation methods.

### BACKGROUND

Human milk represents a complex mixture of carbohydrates, fats, proteins, vitamins, minerals, and trace elements. The by far most predominant fraction is represented by carbohydrates, which can be further divided into lactose and more complex oligosaccharides (Human milk oligosaccharides, HMO). Whereas lactose is used as an energy source, the complex oligosaccharides are not metabolized by the infant. The fraction of complex oligosaccharides accounts for up to 1/10 of the total carbohydrate fraction and consists of probably more than 150 different oligosaccharides. The occurrence and concentration of these complex oligosaccharides are specific to humans and thus cannot be found in large quantities in the milk of other mammals, like for example domesticated dairy animals.

To date, the structures of at least 115 HMOs have been determined, and considerably more are probably present in human milk. HMOs have become of great interest in the last decade, due to the discovery of their important functionality in human development. Besides their prebiotic properties, HMOs have been linked to additional positive effects, which expands their field of application. The health benefits of HMOs have enabled their approval for use in foods, such as infant formulas and foods, and for consumer health products.

To bypass the drawbacks associated with the chemical synthesis of HMOs, several enzymatic methods and fermentative approaches have been developed. Fermentation based processes have traditionally been developed for individual HMOs such as 2′-fucosyllactose (2′-FL), 3-fucosyllactose (3-FL), lacto-N-tetraose (LNT), lacto-N-neotetraose (LNnT), 3′-sialyllactose (3′-SL) and 6′-sialyllactose (6′-SL).

Fermentation based processes typically utilize genetically engineered bacterial strains, such as recombinant Escherichia coli (E. coli).

Biotechnological production, such as a fermentation process, of HMOs is a valuable, cost-efficient, and large-scale approach to HMO manufacturing. It relies on genetically engineered bacteria constructed to express the glycosyltransferases needed for synthesis of the desired oligosaccharides and takes advantage of the bacteria's innate pool of nucleotide sugars as HMO precursors. At present, knowledge as to how to make composition of Lacto-N-fucopentaose I (LNFP-I)-containing blends and how to fine-tune the levels of the different HMOs of the acquired blends, either by genetic engineering, or adjustment of fermentation parameters, is non-existent, because commercial fermentation process parameters for HMO manufacturing are normally kept secret and therefore the effects of e.g., fermentation parameters on LNFP-I blend compositions, or any other HMO blends, has not been described.

WO 2019/0011133 describes the identification of fucosyltransferases that can fucosylate LNT or LNnT to produce LNFP-I, LNFP-II, LNFP-Ill and LNFP-VI. Specifically, the use of FucT fucosyltransferases to produce LNFP-I are described. There is however no disclosure of a fucosyltransferase that can produce blends of LNFP-I and 2′-FL.

WO 2019/123324 describes the formation of LNFP-I, there is however no indication of the molar % of LNFP-I or 2′-FL constituted in the total amount of HMO formed.

### SUMMARY

The present disclosure targets biotechnological production of HMO blends, while the industrial focus normally is on producing pure HMOs, i.e., a typical interest is to minimize HMO by-product levels and purify them away in downstream processes such as by centrifugation.

The present disclosure provides detailed and in-depth knowledge as how to produce specific HMO blends from a genetically engineered cell, with LNFP-I and 2′-FL as the predominant HMOs, out of a broad diversity of possible blend compositions and tailor them to specific markets, customers and to achieve specific biological effects, while knowledge of the biological activity and function of specific HMOs and HMO mixtures is rapidly emerging.

The immediate advantage is that the blends are manufactured by one producer strain and purified as a mixture of HMOs and hence, not mixed from individually purified HMOs produced by several producer stains. This gives a more sustainable manufacturing process; valuable HMOs are not discarded during the purification process and the conversion from carbon source to HMO product in fermentation is thus done at a much higher overall yield.

In its broadest aspect, the present disclosure relates to a method for the production of a human milk oligosaccharide (HMO) blend with LNFP-I and/or 2′-FL as the predominant HMOs, wherein above 60 molar % of the total human milk oligosaccharide (HMO) produced is LNFP-I, the method comprising the steps of
a. providing a genetically engineered cell capable of producing an HMO, wherein said cell
   i. comprises a heterologous β-1,3-N-acetyl-glucosaminyltransferase protein as shown in SEQ ID NO: 1 [lgtA] or 2 [PmnagT] or 3 [HDO466], or a functional homologue thereof having an amino acid sequence which is at least 80% identical to SEQ ID NO: 1, 2 or 3; and
   ii. comprises a heterologous β-1,3-galactosyltransferase protein as shown in SEQ ID NO: 4 [galTK] or 5 [cvb3galT], or a functional homologue thereof having an amino acid sequence which is at least 80% identical to SEQ ID NO: 4 or 5; and
   iii. comprises a heterologous α-1,2-fucosyltransferase protein as shown in any one of SEQ ID NO: 6 [futC] and 7 [mtun] and 49[FucT54] or a functional homologue thereof having an amino acid sequence which is at least 80% identical to SEQ ID NO: 6 and 7 and 498, and
   iv. expresses functionally the colanic acid gene cluster, and
   v. comprises a native or heterologous regulatory element for controlling the expression of any one of i)-iv), and
b. culturing the cell according to (a) in a suitable cell culture medium to express said proteins and to produce an HMO blend; and
c. harvesting the human milk oligosaccharide (HMO) blend produced in step (b).

The regulatory element in v), such as a promoter, controls the expression of the mentioned glycosyltransferases (i-iii) and the colonic acid gene cluster (iv), and this regulatory element should precede the coding sequence of i, ii, iii and/or iv of the construct (promoter/regulatory element+coding sequence). The construct may be integrated into the genome, or it can be introduced into the cell in the form of a plasmid or another episomal element.

A further aspect disclosed herein relates to a genetically engineered cell comprising a recombinant nucleic acid sequence encoding
i. a heterologous β-1,3-N-acetyl-glucosaminyltransferase protein as shown in SEQ ID NO: 1 or 2 or 3, or a functional homologue thereof having an amino acid sequence which is at least 80% identical to SEQ ID NO: 1, 2 or 3; and
ii. a heterologous β-1,3-galactosyltransferase protein as shown in SEQ ID NO: 4 or 5, or a functional homologue thereof having an amino acid sequence which is at least 80% identical to SEQ ID NO: 4 or 5; and
iii. a heterologous α-1,2-fucosyltransferase protein as shown in any one of SEQ ID NO: 6 or 7 or 49 or a functional homologue thereof having an amino acid sequence which is at least 80% identical to any one of SEQ ID NO: 6, 7 or 49, and
iv. the colanic acid gene cluster, and
v. a native or heterologous regulatory or episomal element for controlling the expression of and of i)-iv), and
vi. a recombinant nucleic acid sequence encoding a sugar efflux transporter capable of exporting 2′FL and/or LNFP-I out of the cell.

In a further aspect, the disclosure relates to a nucleic acid construct comprising a nucleic acid sequence encoding one or more of the proteins selected from the group consisting of
i. a heterologous β-1,3-N-acetyl-glucosaminyltransferase protein as shown in SEQ ID NO: 1 [lgtA] or SEQ ID NO: 2 [PmnagT] or SEQ ID NO: 3 [HDO466], or a functional homologue thereof having an amino acid sequence which is at least 80% identical to SEQ ID NO: 1, 2 or 3; and
ii. a heterologous β-1,3-galactosyltransferase protein as shown in SEQ ID NO: 4 [galTK] or SEQ ID NO: 5 [cvb3galT], or a functional homologue thereof having an amino acid sequence which is at least 80% identical to SEQ ID NO: 4 or 5; and
iii. a heterologous α-1,2-fucosyltransferase protein as shown in any one of SEQ ID NO: 6 [futC] or SEQ ID NO: 7 [mtun] or SEQ ID NO: 49 [fucT54] or a functional homologue thereof having an amino acid sequence which is at least 80% identical to any one of SEQ ID NO: 6, 7 or 49, and
iv. expresses functionally the colanic acid gene cluster (gmd, wcaG, wcaH, wcal, manC, manB), and
   said nucleic acid construct further comprises a native or heterologous regulatory element for controlling the expression of the genes present in the nucleic acid construct, i.e. one or more of i)-iv). Preferably the regulatory element is a recombinant promoter sequence derived from the promoter sequence of the lac operon or a g/p operon and one or more of the coding sequence of i) to iv) and the promoter sequence is operably linked.

In another aspect, the disclosure relates to the use of a genetically engineered cell, or a nucleic acid construct according to the present disclosure, for the biosynthetic production of one or more Human Milk Oligosaccharides (HMOs), in particular a human milk oligosaccharide (HMO) blend with LNFP-I and 2′-FL as the predominant HMOs.

### DETAILED DESCRIPTION

Various exemplary embodiments and details are described hereinafter, with reference to the figures and sequences when relevant. It should also be noted that the figures are only intended to facilitate the description of the embodiments. They are not intended as an exhaustive description of the disclosure or as a limitation on the scope of the disclosure. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

### Exemplary Methods

The present disclosure converts E. coli host cells to a genetically engineered cell factory for LNFP-I and 2′-FL production. The genetically engineered cell falls in focus areas in the applied rational genetic engineering program with the purpose to
a) screen for different wild-type α-1,2-fucosyltransferases that show distinct specificity for important substrates in the HMO production process, namely lactose and LNT,
b) introduce sugar transporters, e.g., of the Major Facilitator Superfamily (MFS), that are able to export 2′-FL and/or LNFP-I out of the cell,
c) increase the gene copy number and/or the expression of genes coding the enzymes that are directly involved in the LNFP-I and 2′-FL biosynthetic pathways, including the synthesis of the activated sugars GDP-fucose, UDP-GlcNAc and UDP-Gal (donor sugars), and
d) the decoration of lactose, LNT-II and LNT (acceptor sugars) to form, respectively, LNT-II, 2′-FL, LNT and LNFP-I.

In principle, the rational engineering strategy (c) can be applied in multiple ways:
1) the copy number of the colanic acid gene cluster and/or any of the glycosyltransferase genes encoding an enzyme involved in 2′-FL and/or LNFP-I synthesis can be varied to fine tune the ratio of these two HMOs to a desired level,
2) the same effect can be achieved by varying the expression of a single copy of these genes at the transcriptional or the translational level. In other words, the strength of the promoter driving the expression of the single gene copy and/or the strength of the Shine-Dalgarno sequence defining the ribosomal binding of the corresponding mRNA can be altered in numerous ways in order to fine tune the ratio of 2′-FL and LNFP-I to a desired level,
3) the deletion of regulators that repress the expression of key genes in the HMO production process is another route towards the improvement of HMO titers in the final production cell, or the modification of the relative concentrations of different HMOs of a given blend. Thus, elimination of the GlpR-imposed repression of PglpF-driven gene expression of enzymes catalysing key steps in the biosynthesis of different HMOs can lead to increased levels of their mRNAs and presumably of the enzymes coded by them. The latter can eventually result not only in higher total HMO titers, but also in multiple changes in the relative concentrations of the different HMOs that are present in LNFPI-containing HMO blends.

Thus, a method for the production of a human milk oligosaccharide (HMO) blend with LNFP-I and 2′-FL as the predominant HMOs is disclosed. Preferably, wherein the HMO blend has molar % of 2′-FL between 25% to 70% and LNFP-I between 30% to 60% of the total HMO.

The method comprises providing a genetically engineered cell capable of producing an HMO blend. The genetically engineered cell may comprise a heterologous β-1,3-N-acetyl-glucosaminyltransferase protein as shown in SEQ ID NO: 1 [IgtA gene] or SEQ ID NO: 2 [PmnagT] or SEQ ID NO: 3 [HDO466], or a functional homologue thereof having an amino acid sequence which is at least 80% identical to SEQ ID NO: 1, 2 or 3.

The genetically engineered cell may also comprise a heterologous β-1,3-galactosyltransferase protein as shown in SEQ ID NO: 4 [galTK gene] or SEQ ID NO: 5 [cvb3galT], or a functional homologue thereof having an amino acid sequence which is at least 80% identical to SEQ ID NO: 4 or 5.

The genetically engineered cell may also comprise a heterologous α-1,2-fucosyltransferase protein as shown in any one of SEQ ID NO: 6 [futC] or SEQ ID NO: 7 [mtun] or SEQ ID NO: 49 [fucT54] or SEQ ID NO: 8 [smob] or a functional homologue thereof having an amino acid sequence which is at least 80% identical to any one of SEQ ID NO: 6, 7, 49 or 8.

To obtain a molar % of 2′-FL between 25% to 70% and LNFP-I between 30% to 60% of the total HMO it is preferred to use the heterologous α-1,2-fucosyltransferase of SEQ ID NO: 6 [FutC] or a functional homologue thereof having an amino acid sequence which is at least 80%.

To obtain a molar % of 2′-FL between 40% to 55% and LNFP-I between 40% to 60% of the total HMO it is preferred to use the heterologous α-1,2-fucosyltransferase of SEQ ID NO: 7 [mtun] or SEQ OD NO: 49 [FucT54] a functional homologue thereof having an amino acid sequence which is at least 80%.

The genetically engineered cell according to the method of the present disclosure may furthermore express functionally the colanic acid gene cluster, such as but not limited to gmd, wcaG, wcaH, wcal, manC, manB from its native genomic locus. The genetically engineered cell may comprise a native or heterologous regulatory element for controlling the expression of the colanic acid gene cluster from its native or any other genomic locus of the cell.

The genetically engineered cell can be cultured in a suitable cell culture medium to express said proteins and to produce an HMO blend with LNFP-I and 2′-FL as the predominant HMO's. The HMO blend may be harvested by any means applied in the industrial settings for human milk oligosaccharide (HMO) production.

### Igta/galTK/mtun

In one or more exemplary methods, the method comprising the steps of:
a. providing a genetically engineered cell capable of producing an HMO, wherein said cell
   i. comprises a heterologous β-1,3-N-acetyl-glucosaminyltransferase protein as shown in SEQ ID NO: 1 [IgtA], or a functional homologue thereof having an amino acid sequence which is at least 80% identical to SEQ ID NO: 1; and
   ii. comprises a heterologous β-1,3-galactosyltransferase protein as shown in SEQ ID NO: 4 [galTK], or a functional homologue thereof having an amino acid sequence which is at least 80% identical to SEQ ID NO: 4; and
   iii. comprises a heterologous α-1,2-fucosyltransferase protein as shown in SEQ ID NO: 7 [mtun], or a functional homologue thereof having an amino acid sequence which is at least 80% identical to SEQ ID NO: 7, and
   iv. expresses functionally the colanic acid gene cluster (gmd, wcaG, wcaH, wcal, manC, manB)
   v. comprises a native or heterologous regulatory element for controlling the expression of i)-iv)
b. culturing the cell according to (a) in a suitable cell culture medium to express said proteins and to produce an HMO blend; and
c. harvesting the human milk oligosaccharide (HMO) blend produced in step (b).

### PmnagT/galTK/mtun

In one or more exemplary methods, the method comprising the steps of:
a. providing a genetically engineered cell capable of producing an HMO, wherein said cell
   i. comprises a heterologous β-1,3-N-acetyl-glucosaminyltransferase protein as shown in SEQ ID NO: 2 [PmnagT], or a functional homologue thereof having an amino acid sequence which is at least 80% identical to SEQ ID NO: 2; and
   ii. comprises a heterologous β-1,3-galactosyltransferase protein as shown in SEQ ID NO: 4 [galTK], or a functional homologue thereof having an amino acid sequence which is at least 80% identical to SEQ ID NO: 4; and
   iii. comprises a heterologous α-1,2-fucosyltransferase protein as shown in SEQ ID NO: 7 [mtun], or a functional homologue thereof having an amino acid sequence which is at least 80% identical to SEQ ID NO: 7, and
   iv. expresses functionally the colanic acid gene cluster (gmd, wcaG, wcaH, wcal, manC, manB)
   v. comprises a native or heterologous regulatory element for controlling the expression of i)-iv)
b. culturing the cell according to (a) in a suitable cell culture medium to express said proteins and to produce an HMO blend; and
c. harvesting the human milk oligosaccharide (HMO) blend produced in step (b).

### HDO466/galTK/mtun

In one or more exemplary methods, the method comprising the steps of:
a. providing a genetically engineered cell capable of producing an HMO, wherein said cell
   i. comprises a heterologous β-1,3-N-acetyl-glucosaminyltransferase protein as shown in SEQ ID NO: 3 [HDO466], or a functional homologue thereof having an amino acid sequence which is at least 80% identical to SEQ ID NO: 3; and
   ii. comprises a heterologous β-1,3-galactosyltransferase protein as shown in SEQ ID NO: 4 [galTK], or a functional homologue thereof having an amino acid sequence which is at least 80% identical to SEQ ID NO: 4; and
   iii. comprises a heterologous α-1,2-fucosyltransferase protein as shown SEQ ID NO: 7 [mtun], or a functional homologue thereof having an amino acid sequence which is at least 80% identical to SEQ ID NO: 7, and
   iv. expresses functionally the colanic acid gene cluster (gmd, wcaG, wcaH, wcal, manC, manB)
   v. comprises a native or heterologous regulatory element for controlling the expression of i)-iv)
b. culturing the cell according to (a) in a suitable cell culture medium to express said proteins and to produce an HMO blend; and
c. harvesting the human milk oligosaccharide (HMO) blend produced in step (b).

### HDO466/cvb3galT/mtun

In one or more exemplary methods, the method comprising the steps of:
a. providing a genetically engineered cell capable of producing an HMO, wherein said cell
   i. comprises a heterologous β-1,3-N-acetyl-glucosaminyltransferase protein as shown in SEQ ID NO: 3 [HDO466], or a functional homologue thereof having an amino acid sequence which is at least 80% identical to SEQ ID NO: 3; and
   ii. comprises a heterologous β-1,3-galactosyltransferase protein as shown in SEQ ID NO: 5 [cvb3galT], or a functional homologue thereof having an amino acid sequence which is at least 80% identical to SEQ ID NO: 5; and
   iii. comprises a heterologous α-1,2-fucosyltransferase protein as shown in SEQ ID NO: 7 [mtun], or a functional homologue thereof having an amino acid sequence which is at least 80% identical to SEQ ID NO: 7, and
   iv. expresses functionally the colanic acid gene cluster (gmd, wcaG, wcaH, wcal, manC, manB)
   v. comprises a native or heterologous regulatory element for controlling the expression of i)-iv)
b. culturing the cell according to (a) in a suitable cell culture medium to express said proteins and to produce an HMO blend; and
c. harvesting the human milk oligosaccharide (HMO) blend produced in step (b).

### PmnagT/cvb3galT/mtun

In one or more exemplary methods, the method comprising the steps of:
a. providing a genetically engineered cell capable of producing an HMO, wherein said cell
   i. comprises a heterologous β-1,3-N-acetyl-glucosaminyltransferase protein as shown in SEQ ID NO: 2 [PmnagT], or a functional homologue thereof having an amino acid sequence which is at least 80% identical to SEQ ID NO: 2; and
   ii. comprises a heterologous β-1,3-galactosyltransferase protein as shown in SEQ ID NO: 5 [cvb3galT], or a functional homologue thereof having an amino acid sequence which is at least 80% identical to SEQ ID NO: 5; and
   iii. comprises a heterologous α-1,2-fucosyltransferase protein as SEQ ID NO: 7 [mtun], or a functional homologue thereof having an amino acid sequence which is at least 80% identical to SEQ ID NO: 7, and
   iv. expresses functionally the colanic acid gene cluster (gmd, wcaG, wcaH, wcal, manC, manB)
   v. comprises a native or heterologous regulatory element for controlling the expression of i)-iv)
b. culturing the cell according to (a) in a suitable cell culture medium to express said proteins and to produce an HMO blend; and
c. harvesting the human milk oligosaccharide (HMO) blend produced in step (b).

### IgtA/cvb3galT/mtun

In one or more exemplary methods, the method comprising the steps of:
a. providing a genetically engineered cell capable of producing an HMO, wherein said cell
   i. comprises a heterologous β-1,3-N-acetyl-glucosaminyltransferase protein as shown in SEQ ID NO: 1 [IgtA], or a functional homologue thereof having an amino acid sequence which is at least 80% identical to SEQ ID NO: 1; and
   ii. comprises a heterologous β-1,3-galactosyltransferase protein as shown in SEQ ID NO: 5 [cvb3galT], or a functional homologue thereof having an amino acid sequence which is at least 80% identical to SEQ ID NO: 5; and
   iii. comprises a heterologous α-1,2-fucosyltransferase protein as shown in SEQ ID NO: 7 [mtun], or a functional homologue thereof having an amino acid sequence which is at least 80% identical to SEQ ID NO: 7, and
   iv. expresses functionally the colanic acid gene cluster (gmd, wcaG, wcaH, wcal, manC, manB)
   v. comprises a native or heterologous regulatory element for controlling the expression of i)-iv)
b. culturing the cell according to (a) in a suitable cell culture medium to express said proteins and to produce an HMO blend; and
c. harvesting the human milk oligosaccharide (HMO) blend produced in step (b).

### IgtA/galTK/futC

In one or more exemplary methods, the method comprising the steps of:
a. providing a genetically engineered cell capable of producing an HMO, wherein said cell
   i. comprises a heterologous β-1,3-N-acetyl-glucosaminyltransferase protein as shown in SEQ ID NO: 1 [IgtA], or a functional homologue thereof having an amino acid sequence which is at least 80% identical to SEQ ID NO: 1; and
   ii. comprises a heterologous β-1,3-galactosyltransferase protein as shown in SEQ ID NO: 4 [galTK], or a functional homologue thereof having an amino acid sequence which is at least 80% identical to SEQ ID NO: 4; and
   iii. comprises a heterologous α-1,2-fucosyltransferase protein as shown in SEQ ID NO: 6 [futC], or a functional homologue thereof having an amino acid sequence which is at least 80% identical to SEQ ID NO: 6, and
   iv. expresses functionally the colanic acid gene cluster (gmd, wcaG, wcaH, wcal, manC, manB)
   v. comprises a native or heterologous regulatory element for controlling the expression of i)-iv)
b. culturing the cell according to (a) in a suitable cell culture medium to express said proteins and to produce an HMO blend; and
c. harvesting the human milk oligosaccharide (HMO) blend produced in step (b).

### PmnagT/galTK/futC

In one or more exemplary methods, the method comprising the steps of:
a. providing a genetically engineered cell capable of producing an HMO, wherein said cell
   i. comprises a heterologous β-1,3-N-acetyl-glucosaminyltransferase protein as shown in SEQ ID NO: 2 [PmnagT], or a functional homologue thereof having an amino acid sequence which is at least 80% identical to SEQ ID NO: 2; and
   ii. comprises a heterologous β-1,3-galactosyltransferase protein as shown in SEQ ID NO: 4 [galTK], or a functional homologue thereof having an amino acid sequence which is at least 80% identical to SEQ ID NO: 4; and
   iii. comprises a heterologous α-1,2-fucosyltransferase protein as shown in SEQ ID NO: 6 [futC], or a functional homologue thereof having an amino acid sequence which is at least 80% identical to SEQ ID NO: 6, and
   iv. expresses functionally the colanic acid gene cluster (gmd, wcaG, wcaH, wcal, manC, manB)
   v. comprises a native or heterologous regulatory element for controlling the expression of i)-iv)
b. culturing the cell according to (a) in a suitable cell culture medium to express said proteins and to produce an HMO blend; and
c. harvesting the human milk oligosaccharide (HMO) blend produced in step (b).

### HDO466/galTK/futC

In one or more exemplary methods, the method comprising the steps of:
a. providing a genetically engineered cell capable of producing an HMO, wherein said cell
   i. comprises a heterologous β-1,3-N-acetyl-glucosaminyltransferase protein as shown in SEQ ID NO: 3 [HDO466], or a functional homologue thereof having an amino acid sequence which is at least 80% identical to SEQ ID NO: 3; and
   ii. comprises a heterologous β-1,3-galactosyltransferase protein as shown in SEQ ID NO: 4 [galTK], or a functional homologue thereof having an amino acid sequence which is at least 80% identical to SEQ ID NO: 4; and
   iii. comprises a heterologous α-1,2-fucosyltransferase protein as shown in SEQ ID NO: 6 [futC], or a functional homologue thereof having an amino acid sequence which is at least 80% identical to SEQ ID NO: 6, and
   iv. expresses functionally the colanic acid gene cluster (gmd, wcaG, wcaH, wcal, manC, manB)
   v. comprises a native or heterologous regulatory element for controlling the expression of i)-iv)
b. culturing the cell according to (a) in a suitable cell culture medium to express said proteins and to produce an HMO blend; and
c. harvesting the human milk oligosaccharide (HMO) blend produced in step (b).

### HDO466/cvb3galT/futC

In one or more exemplary methods, the method comprising the steps of:
a. providing a genetically engineered cell capable of producing an HMO, wherein said cell
   i. comprises a heterologous β-1,3-N-acetyl-glucosaminyltransferase protein as shown in SEQ ID NO: 3 [HDO466], or a functional homologue thereof having an amino acid sequence which is at least 80% identical to SEQ ID NO: 3; and
   ii. comprises a heterologous β-1,3-galactosyltransferase 00000000protein as shown in SEQ ID NO: 5 [cvb3galT], or a functional homologue thereof having an amino acid sequence which is at least 80% identical to SEQ ID NO: 5; and
   iii. comprises a heterologous α-1,2-fucosyltransferase protein as shown in SEQ ID NO: 6 [futC], or a functional homologue thereof having an amino acid sequence which is at least 80% identical to SEQ ID NO: 6, and
   iv. expresses functionally the colanic acid gene cluster (gmd, wcaG, wcaH, wcal, manC, manB)
   v. comprises a native or heterologous regulatory element for controlling the expression of i)-iv)
b. culturing the cell according to (a) in a suitable cell culture medium to express said proteins and to produce an HMO blend; and
c. harvesting the human milk oligosaccharide (HMO) blend produced in step (b).

### PmnagT/cvb3galT/futC

In one or more exemplary methods, the method comprising the steps of:
a. providing a genetically engineered cell capable of producing an HMO, wherein said cell
   i. comprises a heterologous β-1,3-N-acetyl-glucosaminyltransferase protein as shown in SEQ ID NO: 2 [PmnagT], or a functional homologue thereof having an amino acid sequence which is at least 80% identical to SEQ ID NO: 2; and
   ii. comprises a heterologous β-1,3-galactosyltransferase protein as shown in SEQ ID NO: 5 [cvb3galT], or a functional homologue thereof having an amino acid sequence which is at least 80% identical to SEQ ID NO: 5; and
   iii. comprises a heterologous α-1,2-fucosyltransferase protein as shown in SEQ ID NO: 6 [futC], or a functional homologue thereof having an amino acid sequence which is at least 80% identical to SEQ ID NO: 6, and
   iv. expresses functionally the colanic acid gene cluster (gmd, wcaG, wcaH, wcal, manC, manB)
   v. comprises a native or heterologous regulatory element for controlling the expression of i)-iv)
b. culturing the cell according to (a) in a suitable cell culture medium to express said proteins and to produce an HMO blend; and
c. harvesting the human milk oligosaccharide (HMO) blend produced in step (b).

### IgtA/cvb3galT/futC

In one or more exemplary methods, the method comprising the steps of:
a. providing a genetically engineered cell capable of producing an HMO, wherein said cell
   i. comprises a heterologous β-1,3-N-acetyl-glucosaminyltransferase protein as shown in SEQ ID NO: 1 [IgtA], or a functional homologue thereof having an amino acid sequence which is at least 80% identical to SEQ ID NO: 1; and
   ii. comprises a heterologous β-1,3-galactosyltransferase protein as shown in SEQ ID NO: 5 [cvb3galT], or a functional homologue thereof having an amino acid sequence which is at least 80% identical to SEQ ID NO: 5; and
   iii. comprises a heterologous α-1,2-fucosyltransferase protein as shown in SEQ ID NO: 6 [futC], or a functional homologue thereof having an amino acid sequence which is at least 80% identical to SEQ ID NO: 6, and
   iv. expresses functionally the colanic acid gene cluster (gmd, wcaG, wcaH, wcal, manC, manB)
   v. comprises a native or heterologous regulatory element for controlling the expression of i)-iv)
b. culturing the cell according to (a) in a suitable cell culture medium to express said proteins and to produce an HMO blend; and
c. harvesting the human milk oligosaccharide (HMO) blend produced in step (b).

### IgtA/galTK/futC/smob

In one or more exemplary methods, the method comprising the steps of:
a. providing a genetically engineered cell capable of producing an HMO, wherein said cell
   i. comprises a heterologous β-1,3-N-acetyl-glucosaminyltransferase protein as shown in SEQ ID NO: 1 [IgtA], or a functional homologue thereof having an amino acid sequence which is at least 80% identical to SEQ ID NO: 1; and
   ii. comprises a heterologous β-1,3-galactosyltransferase protein as shown in SEQ ID NO: 4 [galTK], or a functional homologue thereof having an amino acid sequence which is at least 80% identical to SEQ ID NO: 4; and
   iii. comprises the two heterologous α-1,2-fucosyltransferase protein as shown in SEQ ID NO: 6 [futC] and SEQ ID NO: 8 [smob], or a functional homologue thereof having an amino acid sequence which independently is at least 80% identical to SEQ ID NO: 6 or SEQ ID NO: 8, and
   iv. expresses functionally the colanic acid gene cluster (gmd, wcaG, wcaH, wcal, manC, manB)
   v. comprises a native or heterologous regulatory element for controlling the expression of i)-iv)
b. culturing the cell according to (a) in a suitable cell culture medium to express said proteins and to produce an HMO blend; and
c. harvesting the human milk oligosaccharide (HMO) blend produced in step (b).

### PmnagT/galTK/fucT54

In one or more exemplary methods, the method comprising the steps of:
a. providing a genetically engineered cell capable of producing an HMO, wherein said cell
   i. comprises a heterologous β-1,3-N-acetyl-glucosaminyltransferase protein as shown in SEQ ID NO: 2 [PmnagT], or a functional homologue thereof having an amino acid sequence which is at least 80% identical to SEQ ID NO: 2; and
   ii. comprises a heterologous β-1,3-galactosyltransferase protein as shown in SEQ ID NO: 4 [galTK], or a functional homologue thereof having an amino acid sequence which is at least 80% identical to SEQ ID NO: 4; and
   iii. comprises a heterologous α-1,2-fucosyltransferase protein as shown in SEQ ID NO: 49 [fucT54], or a functional homologue thereof having an amino acid sequence which is at least 80% identical to SEQ ID NO: 49, and
   iv. expresses functionally the colanic acid gene cluster (gmd, wcaG, wcaH, wcal, manC, manB)
   v. comprises a native or heterologous regulatory element for controlling the expression of i)-iv)
b. culturing the cell according to (a) in a suitable cell culture medium to express said proteins and to produce an HMO blend; and
c. harvesting the human milk oligosaccharide (HMO) blend produced in step (b).

### HDO44949/galTK/fucT54

In one or more exemplary methods, the method comprising the steps of:
a. providing a genetically engineered cell capable of producing an HMO, wherein said cell
   i. comprises a heterologous β-1,3-N-acetyl-glucosaminyltransferase protein as shown in SEQ ID NO: 3 [HDO44949], or a functional homologue thereof having an amino acid sequence which is at least 80% identical to SEQ ID NO: 3; and
   ii. comprises a heterologous β-1,3-galactosyltransferase protein as shown in SEQ ID NO: 4 [galTK], or a functional homologue thereof having an amino acid sequence which is at least 80% identical to SEQ ID NO: 4; and
   iii. comprises a heterologous α-1,2-fucosyltransferase protein as shown in SEQ ID NO: 49 [fucT54], or a functional homologue thereof having an amino acid sequence which is at least 80% identical to SEQ ID NO: 49, and
   iv. expresses functionally the colanic acid gene cluster (gmd, wcaG, wcaH, wcal, manC, manB)
   v. comprises a native or heterologous regulatory element for controlling the expression of i)-iv)
b. culturing the cell according to (a) in a suitable cell culture medium to express said proteins and to produce an HMO blend; and
c. harvesting the human milk oligosaccharide (HMO) blend produced in step (b).

### HDO44949/cvb3galT/fucT54

In one or more exemplary methods, the method comprising the steps of:
a. providing a genetically engineered cell capable of producing an HMO, wherein said cell
   i. comprises a heterologous β-1,3-N-acetyl-glucosaminyltransferase protein as shown in SEQ ID NO: 3 [HDO44949], or a functional homologue thereof having an amino acid sequence which is at least 80% identical to SEQ ID NO: 3; and
   ii. comprises a heterologous β-1,3-galactosyltransferase 00000000protein as shown in SEQ ID NO: 5 [cvb3galT], or a functional homologue thereof having an amino acid sequence which is at least 80% identical to SEQ ID NO: 5; and
   iii. comprises a heterologous α-1,2-fucosyltransferase protein as shown in SEQ ID NO: 49 [fucT54], or a functional homologue thereof having an amino acid sequence which is at least 80% identical to SEQ ID NO: 49, and
   iv. expresses functionally the colanic acid gene cluster (gmd, wcaG, wcaH, wcal, manC, manB)
   v. comprises a native or heterologous regulatory element for controlling the expression of i)-iv)
b. culturing the cell according to (a) in a suitable cell culture medium to express said proteins and to produce an HMO blend; and
c. harvesting the human milk oligosaccharide (HMO) blend produced in step (b).

### PmnagT/cvb3galT/fucT54

In one or more exemplary methods, the method comprising the steps of:
a. providing a genetically engineered cell capable of producing an HMO, wherein said cell
   i. comprises a heterologous β-1,3-N-acetyl-glucosaminyltransferase protein as shown in SEQ ID NO: 2 [PmnagT], or a functional homologue thereof having an amino acid sequence which is at least 80% identical to SEQ ID NO: 2; and
   ii. comprises a heterologous β-1,3-galactosyltransferase protein as shown in SEQ ID NO: 5 [cvb3galT], or a functional homologue thereof having an amino acid sequence which is at least 80% identical to SEQ ID NO: 5; and
   iii. comprises a heterologous α-1,2-fucosyltransferase protein as shown in SEQ ID NO: 49 [fucT54], or a functional homologue thereof having an amino acid sequence which is at least 80% identical to SEQ ID NO: 49, and
   iv. expresses functionally the colanic acid gene cluster (gmd, wcaG, wcaH, wcal, manC, manB)
   v. comprises a native or heterologous regulatory element for controlling the expression of i)-iv)
b. culturing the cell according to (a) in a suitable cell culture medium to express said proteins and to produce an HMO blend; and
c. harvesting the human milk oligosaccharide (HMO) blend produced in step (b).

### IgtA/cvb3galT/fucT54

In one or more exemplary methods, the method comprising the steps of:
a. providing a genetically engineered cell capable of producing an HMO, wherein said cell
   i. comprises a heterologous β-1,3-N-acetyl-glucosaminyltransferase protein as shown in SEQ ID NO: 1 [IgtA], or a functional homologue thereof having an amino acid sequence which is at least 80% identical to SEQ ID NO: 1; and
   ii. comprises a heterologous β-1,3-galactosyltransferase protein as shown in SEQ ID NO: 5 [cvb3galT], or a functional homologue thereof having an amino acid sequence which is at least 80% identical to SEQ ID NO: 5; and
   iii. comprises a heterologous α-1,2-fucosyltransferase protein as shown in SEQ ID NO: 49 [fucT54], or a functional homologue thereof having an amino acid sequence which is at least 80% identical to SEQ ID NO: 49, and
   iv. expresses functionally the colanic acid gene cluster (gmd, wcaG, wcaH, wcal, manC, manB)
   v. comprises a native or heterologous regulatory element for controlling the expression of i)-iv)
b. culturing the cell according to (a) in a suitable cell culture medium to express said proteins and to produce an HMO blend; and
c. harvesting the human milk oligosaccharide (HMO) blend produced in step (b).

The present disclosure provides several strain engineering tools and fermentation process adjustments to generate an HMO blend consisting mainly of 2′-FL and LNFP-I. The approaches presented here not only ensure that 2′-FL and LNFP-I are the predominant HMOs of the acquired blend, but each of them favours the synthesis of either of the two HMOs in a unique manner.

For instance, the synthesis of a blend consisting predominantly of LNFP-I and 2′-FL with LNFP-I being the most abundant HMO can be solely achieved by the choice of an appropriate α-1,2-fucosyltransferase, such as the Smob enzyme from Sulfuriflexus mobilis (GenBank ID: WP_126455392.1). Instead, if a blend consisting predominantly of LNFP-I and 2′-FL with 2′-FL being the most abundant HMO is desirable, then the corresponding production strain needs to express the FutC enzyme from Helicobacter pylori (GenBank ID: WP_080473865.1) along with a heterologous MFS transporter selected from a group of transporter proteins as disclosed herein.

Moreover, the expression levels of the colonic acid gene cluster can be adjusted in a sophisticated manner to enable the prevalence of either HMOs in the blend, 2′-FL or LNFP-I, regardless of the glycosyltransferases being expressed by the production strain. For example, modifying the strength of the promoter driving the expression of the colonic acid gene cluster from its native genetic locus is a unique tool for controlling the levels of intracellular GDP-fucose and subsequently the degree of fucosylation of both the internalized lactose and the newly formed LNT, which in turn affects the ratio of 2′-FL and LNFP-I in a manner that is characteristic for the glycosyltransferases or other heterologous proteins being expressed in the production strain.

### Human Milk Oligosaccharide (HMO)

In the context of the disclosure, the term “oligosaccharide” means a saccharide polymer containing a number of monosaccharide units. The number of monosaccharide units are in the range of 3 to 15, such as in the range of 3 to 10, such as in the range of 3 to 6, such as in the range of 3 to 5. In some embodiments, preferred oligosaccharides are saccharide polymers consisting of three or four monosaccharide units, i.e. trisaccharides, tetrasaccharides, pentasaccharides or hexasaccharides. Preferable oligosaccharides of the disclosure are human milk oligosaccharides (HMOs).

The term “human milk oligosaccharide” or “HMO” in the present context means a complex carbohydrate found in human breast milk. The HMOs have a core structure comprising a lactose unit at the reducing end that can be elongated by one or more beta-N-acetyl-lactosaminyl and/or one or more beta-lacto-N-biosyl units, and this core structure can be substituted by an alpha-L-fucopyranosyl and/or an alpha-N-acetyl-neuraminyl (sialyl) moiety.

In this regard, the non-acidic (or neutral) HMOs are devoid of a sialyl residue, and the acidic HMOs have at least one sialyl residue in their structure. The non-acidic (or neutral) HMOs can be fucosylated or non-fucosylated. Examples of such neutral non-fucosylated HMOs include lacto-N-triose 2 (LNT-2) lacto-N-tetraose (LNT), lacto-N-neotetraose (LNnT), lacto-N-neohexaose (LNnH), para-lacto-N-neohexaose (pLNnH), para-lacto-N-hexaose (pLNH) and lacto-N-hexaose (LNH). Examples of neutral fucosylated HMOs include 2′-fucosyllactose (2′-FL), lacto-N-fucopentaose I (LNFP-I), lacto-N-difucohexaose I (LNDFH-I), 3-fucosyllactose (3-FL), difucosyllactose (DFL), lacto-N-fucopentaose II (LNFP-II), lacto-N-fucopentaose III (LNFP-III), lacto-N-difucohexaose III (LNDFH-III), fucosyl-lacto-N-hexaose II (FLNH-II), lacto-N-fucopentaose V (LNFP-V), lacto-N-difucohexaose II (LNDFH-II), fucosyl-lacto-N-hexaose I (FLNH-I), fucosyl-para-lacto-N-hexaose I (FpLNH-I), fucosyl-para-lacto-N-neohexaose II (F-pLNnH II) and fucosyl-lacto-N-neohexaose (FLNnH). Examples of acidic HMOs include 3-sialyllactose (3′-SL), 6′-sialyllactose (6′-SL), 3-fucosyl-3′-sialyllactose (FSL), 3′-O-sialyllacto-N-tetraose a (LST a), fucosyl-LST a (FLST a), 6′-O-sialyllacto-N-tetraose b (LST b), fucosyl-LST b (FLST b), 6′-O-sialyllacto-N-neotetraose (LST c), fucosyl-LST c (FLST c), 3′-O-sialyllacto-N-neotetraose (LST d), fucosyl-LST d (FLST d), sialyl-lacto-N-hexaose (SLNH), sialyl-lacto-N-neohexaose I (SLNH-I), sialyl-lacto-N-neohexaose II (SLNH-II) and disialyl-lacto-N-tetraose (DSLNT).

In the context of the present disclosure lactose is not regarded as an HMO species.

### Human Milk Oligosaccharide (HMO) Blend

The term “blend” or “HMO blend” refers to a mixture of two or more HMOs and/or HMO precursors, such as but not limited to HMOs selected from LNT, LNnT, LNH, LNT-II, LNnH, para-LNH, para-LNnH, 2′-FL, 3FL, DFL, LNFP I, LNDFH-I, LNFP II, LNFP III, LNFP V, F-LNnH, DF-LNH I, DF-LNH II, DF-LNH I, DF-para-LNH, DF-para-LNnH, 3′-SL, 6′-SL, FSL, F-LST a, F-LST b, F-LST c, LST a, LST b, LST c and DSLNT. The HMO blends as described herein are obtained at the end of fermentation and not by mixing purified HMOs or HMOs produced by different fermentation batches. An HMO blend is the composition of HMOs produced during or at the end of fermentation, the HMO blend at the end of fermentation may also be termed the final HMO blend. The blend of HMOs may be subjected to downstream purification, however with the purpose to maintain a blend of HMO's with similar ratios as in the blend of HMOs obtained after fermentation. It is envisioned that no additional HMOs are added to the blend following the purification.

In one or more exemplary embodiments, the “HMO blend” referred to herein relates to a mixture of two or more HMOs and/or HMO precursors selected from the group consisting of LNT, LNT-II, LNnH, para-LNH, 2′-FL, DFL, and LNFP I. Preferably, the HMO blend, or the major components of the HMO blend are produced from a single production strain.

Blend with LNFP-I and 2′-FL as the Predominant HMO's

This disclosure highlights two principal ways of achieving unique and diverse blends of HMOs with LNFP-I and 2′-FL as the predominant HMOs, namely strain engineering strategies and fermentation process strategies. The strain engineering strategies to achieve this goal comprise the manipulation of the following genetic traits of the HMO producer cell
1. Choice of the introduced α-1,2-fucosyltransferase
2. Extent of the expression of the colanic acid gene cluster
3. Extent of the expression or copy number of the β-1,3-N-acetyl-glucosaminyltransferase
4. Introduction of sugar transporter of the Major Facilitator Superfamily (MFS)
5. Change in the regulatory landscape of the cell e.g. deletion of the glpR regulator

The fermentation process strategies in this disclosure include modulation of the fermentation temperature and/or lactose levels in the fermentation broth to achieve a specific HMO blend profile with a given strain derived from strain engineering, in a highly predictable manner.

### Blend Ratios

The HMO products produced by the methods disclosed herein can also be described by their ratios. The “ratio” as described herein is understood as the ratio between two amounts of HMOs, such as but not limited to the amount of one divided by the amount of the other or the amount of one divided by the total amount.

In one embodiment of the invention, following fermentation with a strain described herein the HMO blend has molar % of LNFP-I between 90-30% and 2′-FL between 70-10%, such as molar % of LNFP-I between 90-40% and 2′-FL between 60-10%, such as a molar % of 2′-FL between 25% to 70% and LNFP-I between 30% to 60% of total HMO of total HMO.

The molar % blend ratios supported by fermentation data (temperature modulation) from the Examples shows exemplary HMO blend composition ranges which could be as follows: LNFP-I [47-63], 2′-FL [31-51] and LNT [1-5] relative to the sum of all HMOs, or in the combination LNFP-I/2′-FL/LNT between 63/31/5 and 47/51/1 (all in molar %), such as but not limited to a molar % of LNFP-I between 80-30% and 2′-FL between 70-20%, when modulating the temperature.

In some embodiments of the invention the fermentation is conducted between 25 and 34° C., preferably between 30 to 32° C. and the predominant HMOs in the blend has molar % of LNFP-I between 30-60% and 2′-FL between 40-70%.

In some embodiments of the invention the fermentation is conducted between 25 and 28° C. and the predominant HMOs in the blend has molar % of LNFP-I between 45-55% and 2′-FL between 45-55%.

In some embodiments of the invention the fermentation is conducted between 28 and 34° C. the predominant HMOs in the blend has molar % of LNFP-I between 30-45% and 2′-FL between 55-70%.

For lactose modulation from the Examples shows exemplary the following ranges for the high lactose process in molar %: LNFP-I/HMO [60-68], LNT/HMO [21-27], LNT-II/HMO [6-9], 2′-FL/HMO [4-6]. And the following ranges were found for the low lactose process in molar %: LNFP-I/HMO [66-70], 2′-FL/HMO [25-30], LNT/HMO [3-4], LNT-II/HMO [1-1.5].

Here, e.g., the low lactose process supports the blend with LNFP-I and 2′-FL being the two most abundant HMOs.

Thus, in one or more exemplary embodiments, the molar % of LNFP-I may be between 90-40% and 2′-FL between 60-10%, when modulating the lactose.

Thus, in one or more exemplary embodiments, the molar % of LNFP-I may be between 90-30% and 2′-FL between 70-10%, when modulating the lactose.

In some embodiment the lactose during fermentation is low, such as below 20 g/L, preferably below 15 g/L, such as between 0.5 and 15 g/L, preferably below 10 g/L, such as between 1 and 10 g/L, and the molar % of 2′-FL is above 20%, preferably above 30% of the total HMO in the blend.

In one or more exemplary embodiments, the ratio between LNFP-I/2′-FL/LNT can be 15:8:2, 15:7:2, 15:6:2, or 15:5:2

The ratios below are based on the material produced in regulatory batches, thus in mass ratio, instead of molar ratio.

In one or more exemplary embodiments, the mass ratio between LNFP-I/2′-FL/LNT can be 60:30:1, 40:20:1, 30:15:1, 10:5:1, 8:4:1, 6:3:1.

It is described herein that, the mass ratio between LNFP-I/2′-FL/LNT can be 80:40:1, 80:30:1, 80:20:1, 80:15:1, 80:10:1, 80:5:1, 80:4:1.

In one or more exemplary embodiments, the mass ratio between LNFP-I/2′-FL/LNT can be 60:30:1, 60:30:2, 60:30:3, 60:30:4, 60:30:5.

In one or more exemplary embodiments, the mass ratio between LNFP-I/2′-FL/LNT can be 60:15:1, 60:15:2, 60:15:3, 60:15:4, 60:15:5.

In one or more exemplary embodiments, the mass ratio between LNFP-I/2′-FL/LNT can be within the following ranges [80-50]: [25-15]: [0-5].

In one or more exemplary embodiments, the mass ratio between (LNFP-I+2′-FL)/LNT can be within the following ranges [95-75]: [0-5].

### Functional Enzymes

Several genetic engineering approaches have been applied to change the abundance of the different HMOs being produced by cells that over-express the colanic acid gene cluster and express a functional enzyme selected from the group consisting of a heterologous β-1,3-N-acetyl-glucosaminyltransferase, a β-1,3-galactosyltransferase and an α-1,2-fucosyltransferase.

In general, as seen in the Examples below, we observed that most genetic manipulations affected the ratio between LNFP-I and 2′-FL, and to a lesser extend the relative abundance of precursor sugars, such as LNT and LNT-II, in the final HMO blend. The final HMO blend is understood as the mixture of HMOs produced by the genetically modified cell at the end of fermentation.

Significant differences in the LNFP-I to 2′-FL ratio of the acquired final HMO blends could be initially obtained by simply introducing different heterologous α-1,2-fucosyltransferases from diverse bacterial species.

The testing of different glycosyltransferases of this type resulted in various final HMO blends, where LNFP-I and 2′-FL were the predominant HMOs. The specificity of the heterologous α-1,2-fucosyltransferase for lactose and LNT mainly determined whether LNFP-I or 2′-FL was the most abundant HMO in the blends.

### Choice of the Introduced Functional Enzyme

For instance, the higher specificity of the α-1,2-fucosyltransferase FutC from Helicobacter pylori (GenBank ID: WP_080473865.1, but with two additional amino acids (LG) at the C-terminus or SEQ ID NO: 6) for lactose led to HMO blends, where 2′-FL was the most abundant HMO.

On the contrary, the alpha-1,2-fucosyltransferase Smob enzyme from Sulfuriflexus mobilis (GenBank ID: WP_126455392.1 or SEQ ID NO: 8) resulted in blends, where LNFP-I was the predominant HMO, 2′-FL was the second most abundant HMO and LNT-II was formed only in limited amounts.

There were also enzymes that led to HMO blends with almost equimolar concentrations of LNFP-I and 2′-FL, which is indicative of their almost even specificity for either key substrate, i.e., LNT and lactose.

Examples of such enzymes are the α-1,2-fucosyltransferases FucT54 from Sideroxydans lithotrophicus ES-11 (GenBank ID: WP_013031010.1 or SEQ ID NO: 49) and mtun from Methylobacter tundripaludum (GenBank ID: WP_031437198.1 or SEQ ID NO: 7).

Apart from the choice of α-1,2-fucosyltransferase, several other types of genetic modifications of cells expressing FutC could specifically affect the ratio between LNFP-I and 2′-FL

Specific genetic changes in futC-expressing cells could thus be linked to given HMO blends, where either LNFP-I or 2′-FL was the most abundant HMO and 2′-FL or LNFP-I was, respectively, the second most abundant HMO.

To be able to synthesize HMO blends, the recombinant cell comprises at least one recombinant nucleic acid, which encodes a functional enzyme with glycosyltransferase activity. The glycosylation activity is to be understood as the enzymatic activity that is necessary for synthesizing an oligosaccharide, from an internalized mono- or disaccharide via consecutive glycosylation steps, wherein the internalized acceptor, e.g. lactose, is glycosylated, in a first glycosylation step, to a trisaccharide, then that trisaccharide is glycosylated, in a second glycosylation step, to a tetrasaccharide, and so forth. The glycosylation steps are mediated by respective glycosyltransferases.

In this regard, the phrase “a glycosyltransferase which is able to transfer a glycosyl residue of an activated sugar nucleotide to an intermediate in the biosynthetic pathway to said oligosaccharide from said acceptor” or “a glycosyltransferase which can transfer a glycosyl residue of an activated sugar nucleotide to an intermediate in the biosynthetic pathway to said oligosaccharide from said acceptor” refers to the second, third etc. glycosylation step. The term “an intermediate in the biosynthetic pathway” in relation to an oligosaccharide or more specifically an HMO, is to be understood as an intermediate oligosaccharide or HMO in the reaction steps needed to produce the desired oligosaccharide or HMO.

For example, if lacto-N-tetraose is made from lactose, a first glycosyl transferase (a β-1,3-N-acetyl-glucosaminyl transferase transfers GlcNAc of UDP-GlcNAc to the internalized lactose to form lacto-N-triose II (LNT-II), then a second glycosyl transferase (a β-1,3-galactosyl transferase) transfers Gal of UDP-Gal to the previously formed LNT-II to form Lacto-N-tetraose (LNT), and a third glycosyl transferase (an α-1,2-fucosyltransferase) transfers Fuc of GDP-Fuc to the previously formed LNT thus forming Lacto-N-fucopentaose I (LNFP-I). In this regard, LNT-II and LNT are considered to be intermediates in the biosynthetic pathway to LNFP-I from lactose (see also FIG. 12). The same or a second glycosyl fucosyltransferase can transfer Fuc of GDP-Fuc to LNFP-I to form Lacto-N-difucohexaose I (LNDFH-I).

The glycosyl transferase gene may be integrated into the genome (by chromosomal integration) of the genetically engineered cell, or alternatively, it may be comprised in a construct that may be integrated into the genome of the genetically engineered cell or inserted into a plasmid DNA and expressed as plasmid-borne glycosyl transferases.

If two or more glycosyl transferases are needed for the production of an HMO blend, e.g. 2′-FL or LNFP-I, two or more recombinant nucleic acids encoding different enzymes with glycosyl transferase activity may be integrated in the genome, included in a construct and/or expressed from a plasmid, e.g. an α-1,2-fucosyltransferase (a first recombinant nucleic acid encoding a first glycosyl transferase) potentially in combination with an a β-1,3-N-acetylglucosaminyltransferase (a second recombinant nucleic acid encoding a second glycosyl transferase) and a β-1,3-galactosyltransferase (a third recombinant nucleic acid encoding a third glycosyl transferase) for the production of 2′-FL and/or LNFP-I, where the first, second and third recombinant nucleic acid can independently from each other be integrated chromosomally or in one or more constructs and/or plasmids.

In one or more exemplary embodiments, both the first, second and third recombinant nucleic acids are stably integrated into the chromosome of the genetically engineered cell. The integration can be at one or more sites in the genome of the host cell. If integrated at one genomic site in the host cell the recombinant nucleic acids can either be under the control of a single regulatory element forming an operon or under control of individual regulatory elements. Alternatively, the recombinant nucleic acids can be integrated in several places in the genome of the host cell under the control of individual regulatory elements.

In another presently exemplary embodiment at least one of the first, second and third glycosyltransferase is/are plasmid-borne.

### Heterologous Expression

In the present disclosure, the term heterologous means that a nucleic acid encoding a protein has been introduced into a cell that does not normally make (i.e., express) that protein, such that the cell is capable of expressing the protein and is termed a genetically modified cell. Thus, heterologous refers to the fact that the expressed protein was initially cloned from or derived from a cell type or a species different from the recipient/host cell. The nucleic acid encoding the desired protein must be within a format that encourages the recipient cell to express the cDNA as a protein (i.e., it is put in an expression vector). Methods for transferring foreign genetic material into a recipient cell include transfection and transduction as well as crisper/cas. The choice of recipient cell type is often based on an experimental need to examine the protein's function in detail, and the most prevalent recipients, known as heterologous expression systems, are chosen usually because they are easy to transfer DNA into or because they allow for a simpler assessment of the protein's function.

As shown in the Examples, the order of abundance of the first and second most abundant HMO in the final blend can be inverted from 2′-FL>LNFP-I to LNFP-I>2′-FL, which is achieved by simultaneously increasing the copy number of the IgtA (coding a β-1,3-N-acetyl-glucosaminyltransferase) and galTK (coding a β-1,3-galactosyltransferase) genes in futC-expressing cells.

Heterologous β-1,3-N-acetyl-glucosaminyltransferase

A heterologous β-1,3-N-acetyl-glucosaminyltransferase is any protein which comprises the ability of transferring the GlcNAc of UDP-GlcNAc to lactose. The β-1,3-N-acetyl-glucosaminyltransferase used herein does not originate in the species of the genetically engineered cell i.e. the gene encoding the β-1,3-galactosyltransferase is of heterologous origin. Examples of heterologous β-1,3-N-acetyl-glucosaminyltransferases are LgtA, PmnagT and HDO466, as exemplified in SEQ ID NO: 1, 2 and 3, respectively.

### IgtA

The IgtA gene is a gene encoding a β-1,3-N-acetyl-glucosaminyl-transferase, and homologues of the gene are found in several bacterial species, wherein the gene is involved in the synthesis of the lacto-N-neo-tetraose structural element of the bacterial lipooligosaccharides.

In one or more exemplary embodiments, the IgtA gene is as shown in SEQ ID NO: 40 or is a functional homologue thereof having a nucleic acid sequence which is at least 70% identical to SEQ ID NO: 40, such as at least 75%, 80%, 85%, 90%, 95%, 98% or 100% identical to SEQ ID NO: 40.

In one or more exemplary embodiments, the IgtA gene encodes the protein of SEQ ID NO: 1 or a functional homologue thereof having an amino acid sequence which is at least 70% identical to SEQ ID NO: 1, such as at least 75%, 80%, 85%, 90%, 95%, 98% or 100% identical to SEQ ID NO: 1.

### PmnagT

In one or more exemplary embodiments, the PmnagT gene is as shown in SEQ ID NO: 41, or a functional homologue thereof having a nucleic acid sequence which is at least 70% identical to SEQ ID NO: 41, such as at least 75%, 80%, 85%, 90%, 95%, 98% or 100% identical to SEQ ID NO: 41.

In one or more exemplary embodiments, the PmnagT gene encodes the protein of SEQ ID NO: 2 or a functional homologue thereof having an amino acid sequence which is at least 70% identical to SEQ ID NO: 2, such as at least 75%, 80%, 85%, 90%, 95%, 98% or 100% identical to SEQ ID NO: 2.

### HDO466

In one or more exemplary embodiments, the HDO466 gene is as shown in SEQ ID NO: 42, or a functional homologue thereof having a nucleic acid sequence which is at least 70% identical to SEQ ID NO: 42, such as at least 75%, 80%, 85%, 90%, 95%, 98% or 100% identical to SEQ ID NO: 42.

In one or more exemplary embodiments, the HDO466 gene encodes the protein of SEQ ID NO: 3 or a functional homologue thereof having an amino acid sequence which is at least 70% identical to SEQ ID NO: 3, such as at least 75%, 80%, 85%, 90%, 95%, 98% or 100% identical to SEQ ID NO: 3.

In one or more exemplary embodiments, the heterologous β-1,3-N-acetyl-glucosaminyltransferases and the HMO blends that can be generated using these enzymes are shown in the below matrix.

| | Protein Sequence ID | | |
|---|---|---|---|
| Gene | (GenBank) | Description | HMO example |
| lgtA | WP_033911473.1 | β-1,3-N- | LNT-II, LNT, LNnT, LNFP-I, LNFP-II, LNFP- |
| | | acetylglucosaminyl- | III, LNFP-V, LNFP-VI, LNDFH-I, LNDFH-II, |
| | | transferase | LNDFH-III, pLNH, F-pLNH I, pLNnH, LST a, |
| | | | LST b, LST c, DSLNT |
| HD0466 | WP_010944479.1 | β-1,3-N- | LNT-II, LNT, LNnT, LNFP-I, LNFP-II, LNFP- |
| | | acetylglucosaminyl- | III, LNFP-V, LNFP-VI, LNDFH-I, LNDFH-II, |
| | | transferase | LNDFH-III, pLNH, F-pLNH I, pLNnH, LST a, |
| | | | LST b, LST c, DSLNT |
| PmnagT | WP_014390683.1 | Putative β-1,3-N- | LNT-II, LNT, LNnT, LNFP-I, LNFP-II, LNFP- |
| | | acetylglucosaminyl- | III, LNFP-V, LNFP-VI, LNDFH-I, LNDFH-II, |
| | | transferase | LNDFH-III, pLNH, F-pLNH I, pLNnH, LST a, |
| | | | LST b, LST c, DSLNT |

Heterologous β-1,3-galactosyltransferase

A heterologous β-1,3-galactosyltransferase is any protein that comprise the ability of transferring the Gal of UDP-Gal to a GlcNAc moiety. The β-1,3-galactosyltransferase used herein does not originate in the species of the genetically engineered cell i.e. the gene encoding the β-1,3-galactosyltransferase is of heterologous origin.

Examples of Heterologous β-1,3-galactosyltransferases are GalTK, and Cvb3galT. Exemplified in SEQ ID NO: 4 and 5, Respectively

galTK

In one or more exemplary embodiments, the galTK gene is as shown in SEQ ID NO: 43, or a functional homologue thereof having a nucleic acid sequence which is at least 70% identical to SEQ ID NO: 43, such as at least 75%, 80%, 85%, 90%, 95%, 98% or 100% identical to SEQ ID NO: 43.

In one or more exemplary embodiments, the galTK gene encodes the protein of SEQ ID NO: 4 or a functional homologue thereof having an amino acid sequence which is at least 70% identical to SEQ ID NO: 4, such as at least 75%, 80%, 85%, 90%, 95%, 98% or 100% identical to SEQ ID NO: 4.

cvb3galT

In one or more exemplary embodiments, the cvb3galT gene is as shown in SEQ ID NO: 44, or a functional homologue thereof having a nucleic acid sequence which is at least 70% identical to SEQ ID NO: 44, such as at least 75%, 80%, 85%, 90%, 95%, 98% or 100% identical to SEQ ID NO: 44.

In one or more exemplary embodiments, the cvb3galT gene encodes the protein of SEQ ID NO: 5 or a functional homologue thereof having an amino acid sequence which is at least 70% identical to SEQ ID NO: 5, such as at least 75%, 80%, 85%, 90%, 95%, 98% or 100% identical to SEQ ID NO: 5.

Some non-limiting embodiments of heterologous β-1,3-galactosyltransferases are shown in the below matrix.

| | Protein Sequence ID | | |
|---|---|---|---|
| Gene | (GenBank) | Description | HMO example |
| galTK | homologous to | β-1,3- | LNT-II, LNT, LNFP-I, LNFP-II, LNFP-V, |
| | BD182026.1 | galactosyltransferase | LNDFH-I, LNDFH-II, pLNH, F-pLNH I, |
| | | | LST a, LST b, DSLNT |
| cvb3galT | WP_080969100.1 | β-1,3- | LNT-II, LNT, LNFP-I, LNFP-II, LNFP-V, |
| | | galactosyltransferase | LNDFH-I, LNDFH-II, pLNH, F-pLNH I, |
| | | | LST a, LST b, DSLNT |

Heterologous α-1,2-fucosyltransferase

A α-1,2-fucosylosyl-transferase is responsible for adding a fucose onto the galactose residue of the 0-antigen repeating unit via an α-1,2 linkage. In one or more exemplary embodiments, the heterologous α-1,2-fucosyltransferase protein is any one of SEQ ID NO: 6 or 7 or 8 or a functional homologue thereof having an amino acid sequence which is at least 80% identical to any one of SEQ ID NO: 6, 7 or 8, such as at least 85%, 90%, 95%, 98% or 100% identical to SEQ ID NO: 6, 7 or 8.

Examples of suitable heterologous α-1,2-fucosyltransferase and their effect on HMO blends are shown in the matrix below.

| | Protein Sequence ID | | | Stand-alone |
|---|---|---|---|---|
| Gene | (GenBank) | Description | HMO example | functionality |
| futC | WP_080473865.1 (with | α-1,2- | 2′-FL, DFL, | High 2′-FL levels |
| | two additional amino | fucosyltransferase | LNFP-I | |
| | acids (LG) at the C- | | | |
| | terminus) | | | |
| Mtun | WP_031437198.1 | α-1,2- | 2′-FL, DFL, | Similar 2′-FL and |
| | | fucosyltransferase | LNFP-I | LNFP-I levels |
| FucT54 | WP_013031010.1 | α-1,2- | 2′-FL, DFL, | Similar 2′-FL and |
| | | fucosyltransferase | LNFP-I | LNFP-I levels |
| smob | WP_126455392.1 | α-1,2- | 2′-FL, LNFP-I | High LNFP-I levels |
| | | fucosyltransferase | | |

futC

In one or more exemplary embodiments, the futC gene is as shown in SEQ ID NO: 45, or a functional homologue thereof having a nucleic acid sequence which is at least 70% identical to SEQ ID NO: 45, such as at least 75%, 80%, 85%, 90%, 95%, 98% or 100% identical to SEQ ID NO: 45.

In one or more exemplary embodiments, the futC gene encodes the protein of SEQ ID NO: 6 or a functional homologue thereof having an amino acid sequence which is at least 70% identical to SEQ ID NO: 6, such as at least 75%, 80%, 85%, 90%, 95%, 98% or 100% identical to SEQ ID NO: 6.

### Mtun

In one or more exemplary embodiments, the mtun gene is as shown in SEQ ID NO: 46, or a functional homologue thereof having a nucleic acid sequence which is at least 70% identical to SEQ ID NO: 46, such as at least 75%, 80%, 85%, 90%, 95%, 98% or 100% identical to SEQ ID NO: 46.

In one or more exemplary embodiments, the mtun gene encodes the protein of SEQ ID NO: 7 or a functional homologue thereof having an amino acid sequence which is at least 70% identical to SEQ ID NO: 7, such as at least 75%, 80%, 85%, 90%, 95%, 98% or 100% identical to SEQ ID NO: 7.

### FucT54

In one or more exemplary embodiments, the FucT54 gene encodes the protein of SEQ ID NO: 49 or a functional homologue thereof having an amino acid sequence which is at least 70% identical to SEQ ID NO: 49, such as at least 75%, 80%, 85%, 90%, 95%, 98% or 100% identical to SEQ ID NO: 49. In WO2019/008133, FucT54 has been shown to produce some LNFP-I, however the ability to produce 2′-FL has not been investigated.

smob

The smob gene is as shown in SEQ ID NO: 47, or a functional homologue thereof having a nucleic acid sequence which is at least 70% identical to SEQ ID NO: 47, such as at least 75%, 80%, 85%, 90%, 95%, 98% or 100% identical to SEQ ID NO: 47.

The smob gene encodes the protein of SEQ ID NO: 8 or a functional homologue thereof having an amino acid sequence which is at least 70% identical to SEQ ID NO: 8, such as at least 75%, 80%, 85%, 90%, 95%, 98% or 100% identical to SEQ ID NO: 8.

The heterologous α-1,2-fucosyltransferase protein is SEQ ID NO: 8 [smob]. This is particularly useful when a higher level of LNFP-I is desired, and the cell may optimally further comprise a gene product that acts as a sugar transporter.

In one or more exemplary embodiments, the heterologous α-1,2-fucosyltransferase protein is SEQ ID NO: 7 [mtun] or SEQ ID NO: 49 [fucT54]. These are particularly useful HMO blends with if equimolar concentrations of LNFP-I and 2′-FL is desired.

In one or more exemplary embodiments, the heterologous α-1,2-fucosyltransferase protein is SEQ ID NO: 6 [futC]. This is particularly useful when a higher level of 2′FL is desired and the cell further comprises a gene product that acts as a sugar transporter.

### Colanic Acid Gene Cluster

The colanic acid gene cluster of Escherichia coli K-12 is responsible for production of the extracellular polysaccharide colanic acid, a major oligosaccharide of the bacterial cell wall. The colonic acid (CA) gene cluster is composed of the following genes, gmd, wcaG, wcaH, wcal, manB and manC which also contribute to the GDP-fucose biosynthetic pathway, which is important in the generation of HMO's since GDP-fucose acts as a donor for the fucosylation of the glycosyl units in the HMO. An example of the CA gene cluster is shown in SEQ ID NO: 52.

Since the colanic acid gene cluster as well as the heterologous genes that encode a β-1,3-N-acetyl-glucosaminyltransferase, a β-1,3-galactosyltransferase and an α-1,2-fucosyltransferase were introduced in the genetically modified cells used herein, in the form of PglpF-driven expression cassettes, the deletion of the glpR gene (which codes the DNA-binding transcriptional repressor GlpR) could eliminate the GlpR-imposed repression of transcription from all PglpF promoters in the cell and in this manner enhance gene expression from all PglpF-based cassettes. Thus, the HMO content of the final blend can be affected in multiple ways. In the framework of the present disclosure, it was observed that deleting the glpR gene from the genetic background of futC-expressing cells can increase the LNFP-I to 2′-FL ratios in the final HMO blend.

In one or more exemplary embodiments, the colanic acid gene cluster may be expressed from its native genomic locus to produce functional proteins encoded by the gene cluster thereby contributing to the GDP-fucose biosynthetic pathway. The expression may be actively modulated. The expression can be modulated by swapping the native promoter with a promoter of interest, and/or increasing the copy number of the colanic acid genes coding said protein(s) by expressing the gene cluster from another genomic locus, or episomally expressing the colanic acid gene cluster. As shown in the Examples such means improves the function of e.g., the heterologous α-1,2-fucosyltransferase protein, see Example 2.

Thus, in one or more exemplary embodiments, the expression of the colanic acid gene cluster in iv) is modulated by swapping the native promoter with a promoter of interest, and/or increasing the copy number of the colanic acid genes coding said protein(s) by expressing the gene cluster from another genomic locus, or episomally expressing the colanic acid gene cluster.

In one or more exemplary embodiments, the colanic acid gene cluster in iv) may be expressed functionally.

### Expresses Functionally

In the present context, the term “expresses functionally” in relation to the colanic acid gene cluster should be understood as follows: the expression of the colanic acid gene cluster should provide the enzymes required for a functional GDP-fucose biosynthetic pathway.

The expression can be modulated by swapping the native promoter with a promoter of interest. The expression can also be modulated by increasing the copy number of the colanic acid genes encoding said protein(s). Episomally expressing the colanic acid gene cluster also affects the expression.

Thus, in one or more exemplary embodiments, the expression of the colanic acid gene cluster in v) is modulated by swapping the native promoter with a promoter of interest, and/or increasing the copy number of the colanic acid genes encoding said protein(s), or episomally expressing the colanic acid gene cluster.

In one or more exemplary embodiments, controlling the expression of the colanic acid gene cluster is modulated by swapping the native promoter with a promoter of interest, and/or increasing the copy number of the colanic acid genes coding said protein(s) by expressing the cluster from a different locus on the chromosome, or episomally expressing the colanic acid gene cluster. The individual genes in the CA gene cluster are described below.

### Gmd

The gmd gene encodes the protein GDP-mannose-4,6-dehydratase (UniProt accession nr P0AC88), which catalyzes the conversion of GDP-D-mannose to GDP-4-dehydro-6-deoxy-D-mannose. The protein is involved in the reaction that synthesizes GDP-L-fucose from GDP-alpha-D-mannose. In one or more exemplary embodiments, the gmd gene is over-expressed.

wcaG

The wcaG gene, also known as fcl, encodes the protein GDP-L-fucose synthase (EC 1.1.1.271, (UniProt accession nr P32055), which catalyses the two-step NADP-dependent conversion of GDP-4-dehydro-6-deoxy-D-mannose to GDP-fucose, involving an epimerase and a reductase reaction.

In one or more exemplary embodiments, the wcaG gene is over-expressed.

wcaH

The wcaH gene encodes the protein GDP-mannose mannosyl hydrolase (EC 3.6.1.-, (UniProt accession nr P32056), that hydrolyzes both GDP-mannose and GDP-glucose.

In one or more exemplary embodiments, the wcaH gene is over-expressed.

wcal

The wcal gene encodes the colanic acid biosynthesis glycosyltransferase Wcal (UniProt accession nr P32057), and it catalyses the transfer of unmodified fucose to UPP-Glc (α-D-glucopyranosyl-diphosphoundecaprenol-glucose).

In one or more exemplary embodiments, the wcal gene is over-expressed.

manB

The manB gene encodes the protein phosphomannomutase (EC 5.4.2.8, (UniProt accession nr P24175), which is involved in the biosynthesis of GDP-mannose by catalysing conversion α-D-mannose-1-phosphate into D-mannose-6-phosphate. Thus, the expression level of manB regulates the formation of GDP-mannose.

In one or more exemplary embodiments, the manB gene is over-expressed.

manC

The manC gene encodes the protein mannose-1-phosphate guanylyltransferase (EC:2.7.7.13, (UniProt accession nr P24174), that is involved in the biosynthesis of GDP-mannose through synthesis of GDP-mannose from GTP and α-D-mannose-1-phosphate.

In one or more exemplary embodiments, the manC gene is over-expressed.

### Native Genomic Locus

In relation to the present disclosure, the term “native genomic locus”, in relation to the colanic acid gene cluster, relates to the original and natural position of the gene cluster in the genome of the genetically engineered cell.

### Sequence Identity

The term “sequence identity of [a certain] %” in the context of two or more nucleic acid or amino acid sequences means that the two or more sequences have nucleic acids or amino acid residues in common in the given percent, when compared and aligned for maximum correspondence over a comparison window or designated sequences of nucleic acids or amino acids (i.e. the sequences have at least 90 percent (%) identity). Percent identity of nucleic acid or amino acid sequences can be measured using a BLAST 2.0 sequence comparison algorithm with default parameters, or by manual alignment and visual inspection (see e.g. http://www.ncbi.nlm.nih.gov/BLAST/). This definition also applies to the complement of a test sequence and to sequences that have deletions and/or additions, as well as those that have substitutions. An example of an algorithm that is suitable for determining percent identity, sequence similarity and for alignment is the BLAST 2.2.20+ algorithm, which is described in Altschul et al. Nucl. Acids Res. 25, 3389 (1997). BLAST 2.2.20+ is used to determine percent sequence identity for the nucleic acids and proteins of the disclosure. Software for performing BLAST analyses is publicly available through the National Center for Biotechnology Information (http://www.ncbi.nlm.nih.gov/). Examples of commonly used sequence alignment algorithms are
CLUSTAL Omega (http://www.ebi.ac.uk/Tools/msa/clustalol),
EMBOSS Needle (http://www.ebi.ac.uk/Tools/psa/emboss_needle/),
MAFFT (http://mafft.cbrc.jp/alignment/server/), or
MUSCLE (http://www.ebi.ac.uk/Tools/msa/muscle/).

Preferably, the sequence identity between two amino acid sequences is determined using the Needleman-Wunsch algorithm (Needleman and Wunsch, 1970, J. Mo/. Biol. 48: 443-453) as implemented in the Needle program of the EMBOSS package (EMBOSS: The European Molecular Biology Open Software Suite, Rice et al., 2000, Trends Genet. 16: 276-277,), preferably version 5.0.0 or later (available at https://www.ebi.ac.uk/Tools/psa/emboss needle/). The parameters used are gap open penalty of 10, gap extension penalty of 0.5, and the EBLOSUM62 (EMBOSS version of 30 BLOSUM62) substitution matrix. The output of Needle labeled “longest identity” (obtained using the -nobrief option) is used as the percent identity and is calculated as follows: (Identical Residues×100)/(Length of Alignment−Total Number of Gaps in Alignment).

Preferably, the sequence identity between two nucleotide sequences is determined using the Needleman-Wunsch algorithm (Needleman and Wunsch, 1 970, supra) as implemented in the Needle program of the EMBOSS package (EMBOSS: The European Molecular Biology Open Software Suite, Rice et al., 2000, Trends Genet. 16: 276-277), 10 preferably version 5.0.0 or later. The parameters used are gap open penalty of 10, gap extension penalty of 0.5, and the DNAFULL (EMBOSS version of NCBI NUC4.4) substitution matrix. The output of Needle labeled “longest identity” (obtained using the-nobrief option) is used as the percent identity and is calculated as follows: (Identical Deoxyribonucleotides×100)/(Length of Alignment−Total Number of Gaps in Alignment).

### Functional Homologue

A functional homologue of a protein/nucleic acid sequence as described herein is a protein/nucleic acid sequence with alterations in the genetic code, which retain its original functionality. A functional homologue may be obtained by mutagenesis. The functional homologue should have a remaining functionality of at least 50%, such as 60%, 70%, 80%, 90% or 100% compared to the functionality of the protein/nucleic acid sequence.

A functional homologue of any one of the disclosed amino acid or nucleic acid sequences can also have a higher functionality. A functional homologue of any one of SEQ ID NOs: 1-47, should ideally be able to participate in the HMO production, in terms of HMO yield, purity, reduction in biomass formation, viability of the genetically engineered cell, robustness of the genetically engineered cell according to the disclosure, or reduction in consumables.

### Controlling the Expression

In the present context the term “controlling the expression” relates to gene expression where the transcription of a gene into mRNA and its subsequent translation into protein is controlled. Gene expression is primarily controlled at the level of transcription, largely as a result of binding of proteins to specific sites on DNA, such as but not limited to regulatory elements.

As described above, engineering strategy can be applied in multiple ways by:
1) the copy number
2) controlling the expression of any copy of these genes at the transcriptional or the translational level
3) deletion of regulators that repress the expression of key genes in the HMO production process
4) the over-expression of regulators that activate and/or enhance the expression of key genes in the HMO production process

Increasing the gene copy number and/or the expression of genes coding the enzymes that are directly involved in the LNFP-I and 2′-FL biosynthetic pathways, including the synthesis of the activated sugars GDP-fucose, UDP-GlcNAc and UDP-Gal (donor sugars) and the decoration of lactose, LNT-II, LNT and LNFP-I (acceptor sugars) to form, respectively, LNT-II, 2′-FL, LNT, LNFP-I and LNDFH-I is desired.

### Over-Expression

A variety of molecular mechanisms ensures that genes are expressed at the appropriate level and under conditions of relevance to the applied production process. For instance, the regulation of transcription can be summarized into the following routes of influence; genetic (direct interaction of a control factor with the gene of interest), modulation and/or interaction of a control factor with the transcriptional machinery and epigenetic (non-sequence changes in DNA structure that influence transcription).

It is known that a reduction in gene expression below a critical threshold for any gene will result in a mutant phenotype, since such a defect essentially mimics either a partial or complete loss of function of the target gene, whereas increased expression of a native gene can be both beneficial or disruptive to a cell or organism.

Over-expression of a gene may be achieved directly by transcriptional activators that bind to key gene regulatory sequences to promote transcription or enhancers that constitute sequence elements positively affecting transcription, also termed regulatory elements as described below. Similarly, direct over-expression of a gene can be achieved by simply increasing its copy number in the genome, or replacing its native promoter with a promoter of higher strength or even modifying the sequence controlling the binding of the corresponding mRNA to the ribosomes, i.e. the Shine-Dalgarno sequence being present upstream of the gene's coding sequence.

Moreover, over-expression of a gene may also be achieved indirectly through the partial or full inactivation of transcriptional repressors that normally bind key regulatory sequences around the coding sequence of the gene of interest and thereby inhibit its transcription.

Thus, in one or more exemplary embodiments, the over-expression of the heterologous β-1,3-N-acetyl-glucosaminyltransferase, β-1,3-galactosyltransferase and α-1,2-fucosyltransferase protein(s) in steps i), ii) and iii) in the methods described herein is provided by increasing the copy number of the genes coding said protein(s) and/or by choosing an appropriate element for controlling the expression of these genes and/or inactivating a repressor that binds to regulatory elements around the coding sequences of the genes coding said protein(s).

### Increasing the Copy Number

Copy number variation is a type of structural variation: specifically, it is a type of multiplication of a considerable number of base pairs, which if representing a protein encoding gene will result in an increase of the number of genes encoding the same protein. Such variation can occur naturally in many species but can also be introduced by genetically modifying a host cell.

In one or more exemplary embodiments, expression is controlled by increasing the copy number of the desired gene(s). Copy numbers can be increased either by introducing a plasmid which has a high copy number in the cell or by introducing an additional copy of the gene into the genome of the host cell.

For example, the final blend can be inverted from 2′-FL>LNFP-I to LNFP-I>2′-FL, by simultaneously increasing the copy number of the IgtA (coding a β-1,3-N-acetyl-glucosaminyltransferase) and galTK (coding a β-1,3-galactosyltransferase) genes in futC-expressing cells, see Example 3.

Thus, in one or more exemplary embodiments, the aim is to increase the copy number of the genes coding the β-1,3-N-acetyl-glucosaminyltransferase and the β-1,3-galactosyltransferase, exemplified e.g. by IgtA and galTK, in combination with the α-1,2-fucosylosyl-transferase of SEQ ID NO: 6 [FutC] or SEQ ID NO: 7 [mtun] or SEQ ID NO: 49 [FucT54] or a homologue thereof.

### Regulatory Element

The genetically engineered cell according to the methods described herein may comprise regulatory elements enabling the controlled over-expression of endogenous or heterologous and/or synthetic nucleic acid sequences.

The term “regulatory element”, comprises promoter sequences, signal sequence, and/or arrays of transcription factor binding sites that affect transcription and/or translation of a nucleic acid sequence operably linked to the regulatory element.

Regulatory elements are found at transcriptional and post-transcriptional levels and further enable molecular networks at those levels. For example, at the post-transcriptional level, the biochemical signals controlling mRNA stability, translation and subcellular localization are processed by regulatory elements. RNA binding proteins are another class of post-transcriptional regulatory elements and are further classified as sequence elements or structural elements. Specific sequence motifs that may serve as regulatory elements are also associated with mRNA modifications. A variety of DNA regulatory elements are involved in the regulation of gene expression and rely on the biochemical interactions involving DNA, the cellular proteins that make up chromatin, gene activators and repressors, and transcription factors.

In general, the transcriptional and translational regulatory sequences include, but are not limited to, promoter sequences, ribosomal binding sites, transcriptional start and stop sequences, translational start and stop sequences, binding sites for gene regulators and enhancer sequences.

Promoters and enhancers are the primary genomic regulatory components of gene expression. Promoters are DNA regions within 1-2 kilobases (kb) of a gene's transcription start site (TSS); they contain short regulatory elements (DNA motifs) necessary to assemble RNA polymerase transcriptional machinery. In bacterial and archaeal species is common to have a Shine-Dalgarno sequence downstream of the promoter, typically around 8 bases from the start codon. In addition, DNA regulatory elements located more distal to the TSS can contribute significantly to transcription. Such regions, often termed enhancers, are position-independent DNA regulatory elements that interact with site-specific transcription factors to establish cell type identity and regulate gene expression. Enhancers may act independently of their sequence context and at distances of several to many hundreds of kb from their target genes through a process known as looping. Because of these features, it is difficult to identify suitable enhancers and link them to their target genes based on DNA sequence alone.

The promoter, together with other transcriptional and translational regulatory nucleic acid sequences (also termed “control sequences”) is necessary to express a given gene or group of genes (an operon).

Identification of suitable promoter sequences that promotes expression of the specific gene of interest is a tedious task, which in many cases require laborious efforts. In relation to the present disclosure regulatory elements may or may not be post-translational regulators or it may or may not be translational regulators.

By choosing an appropriate regulatory element (e.g. promoter, enhancers and/or Shine-Dalgarno sequence) it is possible to affect the expression of a heterologous gene. The strength of a regulatory element, such as a promoter or Shine-Dalgarno sequence can be assed using a lacZ enzyme assay where β-galactosidase activity is assayed as described previously (see e.g. Miller J. H. Experiments in molecular genetics, Cold spring Harbor Laboratory Press, N Y, 1972). Briefly the cells are diluted in Z-buffer and permeabilized with sodium dodecyl sulfate (0.1%) and chloroform. The assays is performed at 30° C. Samples are preheated, the assay initiated by addition of 200 μl ortho-nitro-phenyl-β-galactosidase (4 mg/ml) and stopped by addition of 500 μl of 1 M Na₂CO₃ when the sample had turned slightly yellow. The release of ortho-nitrophenol is subsequently determined as the change in optical density at 420 nm. The specific activities are reported in Miller Units (MU) [A420/(min*ml*A600)]. A regulatory element with an activity above 10,000 MU is considered strong and a regulatory element with an activity below 3,000 MU is considered weak, what is in between has intermediate strength. An example of a strong regulatory element is the PglpF promoter with an activity of approximately 14.000 MU and an example of a weak promoter is Plac which when induced with IPTG has an activity of approximately 2300 MU.

Thus, in one embodiment of the invention the regulatory element comprises one or more elements capable of enhancing the expression, i.e. over-expression of the one or more heterologous nucleic acid sequence(s) according to the invention. In particular, the regulation of the expression levels of β-1,3-N-acetyl-glucosaminyltransferase and/or β-1,3-galactosyltransferase can affect the formation of 2′-FL. In one embodiment regulatory elements of more than 10,000 MU, such as more than 12,000 MU, such as more than 15,000 MU is controlling the expression of 1,3-N-acetyl-glucosaminyltransferase and/or β-1,3-galactosyltransferase. Using strong promoters to control the expression of 1,3-N-acetyl-glucosaminyltransferase and/or β-1,3-galactosyltransferase will result in an increase ratio of LNFP-I to 2′-FL, since this will drive the pathway towards LNFP-I (See FIG. 12).

In another embodiment of the invention the regulatory element comprises one or more elements allowing appropriate control of the expression of the one or more heterologous nucleic acid sequence(s) according to the invention. In particular, the regulation of the expression levels of β-1,3-N-acetyl-glucosaminyltransferase and/or β-1,3-galactosyltransferase can affect the formation of 2′-FL. In one embodiment regulatory elements of less than 10,000 MU, such as less than 8,000 MU, such as less than 6,000 MU such as less than 4,000 MU such as less than 2,000 MU is controlling the expression of 1,3-N-acetyl-glucosaminyltransferase and/or β-1,3-galactosyltransferase. Using promoters of intermediate or weak strength to control the expression of 1,3-N-acetyl-glucosaminyltransferase and/or β-1,3-galactosyltransferase will result in a decreased ratio of LNFP-I to 2′-FL, since this reduces the rate at which the intermediate products LNT-II and LNT are produced thereby allowing more 2′-FL to be produced from the lactose present in the system (See FIG. 12).

In that regard the regulatory element, regulating the expression of nucleic acid sequences and/or genes encoding one or more glycosyltransferases and/or sucrose-hydrolyzing enzymes, and/or a PTS-dependent sucrose utilization system and/or one or more native or heterologous MFS transporter proteins according to the invention, may be a promoter sequence.

In carrying out the methods as disclosed herein, different, or identical promoter sequences may be used to drive transcription of different genes of interest integrated in the genome of the host cell or on episomal DNA.

### Native

In relation to the present disclosure, the term “native” refers to nucleic acid sequences originating from the genome of the original, genetically engineered cell according to the method of the invention, but prior to any genetic modification. In that regard a nucleic acid sequence may be considered native if it originates from the E. coli K-12 strain, is not of heterologous origin and not a recombined nucleic acid sequence, with respect to the genetically engineered cell.

### Heterologous Regulatory Element

A regulatory element may be endogenous or heterologous, and/or recombinant and/or synthetic nucleic acid sequences. In the present context, the term “heterologous regulatory element” is to be understood as a regulatory element that is not endogenous to the original, genetically engineered cell described herein.

The heterologous regulatory element may also be a recombinant regulatory element, wherein two or more non-operably linked native regulatory element(s) are recombined into a heterologous and/or synthetic regulatory element. The heterologous regulatory element, may be introduced into the genetically engineered cell using methods known to the person skilled in the art.

### Promoter Sequences

The regulatory element or elements regulating the expression of the genes and/or nucleic acid sequence(s), may comprise one or more promoter sequence(s), wherein the promoter sequence, is operably linked to the nucleic acid sequence of the gene of interest in that sense regulating the expression of the nucleic acid sequence of the gene of interest.

In one or more exemplary embodiments, the heterologous regulatory element is a promoter sequence.

In general, a promoter may comprise native, heterologous and/or synthetic nucleic acid sequences, and may be a recombinant nucleic acid sequence, recombining two or more nucleic acid sequences or same or different origin as described above, thereby generating a homologous, heterologous or synthetic nucleic promoter sequence, and/or a homologous, heterologous or synthetic nucleic regulatory element.

In one or more exemplary embodiments, the regulatory element of the genes and/or heterologous nucleic acid sequences of the genetically engineered cell comprises more than one native or heterologous promoter sequence.

In one or more exemplary embodiments, the regulatory element of the genetically engineered cell comprises a single promoter sequence.

In one or more exemplary embodiments, the regulatory element of the genes and/or heterologous nucleic acid sequences of the genetically engineered cell comprises two or more regulatory elements with identical promoter sequences.

In one or more exemplary embodiments, regulatory element of the genes and/or heterologous nucleic acid sequences of the genetically engineered cell comprises two or more regulatory elements with non-identical promoter sequences.

The regulatory architectures i.e., gene-by-gene distributions of transcription-factor-binding sites and identities of the transcription factors that bind those sites can be used multiple different growth conditions and there are more than 100 genes from across the E. coli genome, which acts as regulatory elements.

Thus, any promoter sequence enabling transcription and/or regulation of the level of transcription, of one or more heterologous or native nucleic acid sequences that encode one or more proteins as described herein may be suitable.

In one or more exemplary embodiments, the regulatory element is selected from the group consisting of PBAD, Pxyl, PsacB, PxylA, PrpR, PnitA, PT7, Ptac, PL, PR, PnisA, Pb, Pscr, PgatY_70UTR, PglpF, PglpF_SD1, PglpF_SD10, PglpF_SD2, PglpF_SD3, PglpF_SD4, PglpF_SD5, PglpF_SD6, PglpF_SD7, PglpF_SD8, PglpF_SD9, PglpF_B28, PglpF_B29, Plac_16UTR, Plac, PmglB_70UTR and PmglB_70UTR_SD4.

A wide selection of promoter sequences derived from the PglpF, PlgpA, PlgpT, PgatY, PmglB and Plac promoter systems are described in detail WO2019123324 and WO2020255054 (hereby incorporated by reference).

In one or more exemplary embodiments, the regulatory element is a promoter selected from the group consisting of SEQ ID NO: 13 (PglpF), SEQ ID NO: 12 (PgatY_70UTR), SEQ ID NO: 27 (Plac), SEQ ID NO: 9 (PmglB_70UTR), SEQ ID NO: 11 (Pscr), or a variant thereof. In particular, a variant of PglpF or Plac as described in WO2019123324 or a variant of PmglB_70UTR as described in WO2020255054 is desired.

In one or more exemplary embodiments, wherein the expression of the heterologous β-1,3-N-acetyl-glucosaminyltransferase and/or heterologous β-1,3-galactosyltransferase is obtained from a single copy and/or the regulatory element for expression of the heterologous β-1,3-N-acetyl-glucosaminyltransferase and/or heterologous β-1,3-galactosyltransferase has low or intermediate strength. The regulatory element with medium or low strength can be selected from the group consisting of PglpF_SD9 (SEQ ID NO: 23), PglpF_SD7 (SEQ ID NO: 21), PglpF_SD6 (SEQ ID NO: 20), PglpF_B28 (SEQ ID NO: 24), PglpF_B29 (SEQ ID NO: 25), Pscr (SEQ ID NO: 11 and Plac (SEQ ID NO: 27).

In one or more exemplary embodiments, wherein the expression of i) and/or ii) is obtained from two or more copies and/or the regulatory element for expression of i) and/or ii) has high strength. The regulatory element with high strength can be is selected from the group consisting of PglpF (SEQ ID NO: 13) PglpF_SD10 (SEQ ID NO: 15), PglpF_SD8 (SEQ ID NO: 22), PglpF_SD5 (SEQ ID NO: 19), PglpF_SD4 (SEQ ID NO: 18), PgatY_70UTR (SEQ ID NO: 12), PmglB_70UTR (SEQ ID NO: 9) and PmglB_70UTR_SD4 (SEQ ID NO: 9).

In one or more exemplary embodiments, the regulatory element is selected from the group consisting of Pscr, PgatY 70UTR, PglpF, PglpF_SD1, PglpF_SD2, PglpF_SD3, PglpF_SD4, PglpF_SD5, PglpF_SD6, PglpF_SD7, PglpF_SD8, PglpF_SD9, PglpF_SD10, PglpF_B28, PglpF_B29, Plac and Plac_16UTR.

In one or more exemplary embodiments, the regulatory element is selected from the group consisting of PglpF, Pscr, Plac, PglpF_B29, and PglpF_B28.

In a preferred exemplary embodiment of the invention, the promoter sequence comprised in the regulatory element for the regulation of the expression of the genes and/or heterologous nucleic acid sequences of the genetically engineered cell, encompasses the glpFKX operon promoter sequence, PglpF.

In one or more exemplary embodiments, the regulatory element is selected from the group consisting PglpF (SEQ ID NO: 13) or a variant thereof selected from PglpF_SD10 (SEQ ID NO: 15), PglpF_SD9 (SEQ ID NO: 23), PglpF_SD8 (SEQ ID NO: 22), PglpF_SD7 (SEQ ID NO: 21), PglpF_SD6 (SEQ ID NO: 20), PglpF_SD5 (SEQ ID NO: 19), PglpF_SD4 (SEQ ID NO: 18), PglpF_B28 (SEQ ID NO: 24) and PglpF_B29 (SEQ ID NO: 25).

In one or more exemplary embodiments, the promoter sequence comprised in the regulatory element for the regulation of the expression of the genes and/or heterologous nucleic acid sequences of the genetically engineered cell, encompasses the lac operon promoter sequence, Plac.

In one or more exemplary embodiments, the genetically engineered cell originates from the MDO strain (see “materials and methods”) and over-expresses a nucleic acid encoding the colanic acid gene cluster by a simple promoter swapping in front of the native colanic acid locus and/or the introduction of a second copy of this gene cluster at a different genomic locus [Plac(CA)::PglpF_B28 (CA)].

In one or more exemplary embodiments, the regulatory element for the regulation of the expression of a recombinant gene included in the construct of the disclosure is the mg/BAC; galactose/methyl-galactoside ABC transporter periplasmic binding protein promoter PmglB or variants thereof such as but not limited to PmglB_70UTR of SEQ ID NO: 9, or PmglB_70UTR_SD4 of SEQ ID NO: 10. Further PmglB variants are described in as described in WO2020255054.

In one or more exemplary embodiments, the regulatory element for the regulation of the expression of a recombinant gene included in the construct of the disclosure is the gatYZABCD; tagatose-1,6-bisP aldolase promoter PgatY or variants thereof.

### Pscr

In one or more exemplary embodiments, the heterologous regulatory element is Pscr or variants thereof such as but not limited to SEQ ID NO: 11.

### PgatY_70UTR

In one or more exemplary embodiments, the heterologous regulatory element is PgatY 70UTR or variants thereof such as but not limited to SEQ ID NO: 12.

### PglpF

In one or more exemplary embodiments, the heterologous regulatory element is PglpF or variants thereof such as but not limited to SEQ ID NO: 13.

### PglpF_SD1

In one or more exemplary embodiments, the heterologous regulatory element is PglpF_SD1 or variants thereof such as but not limited to SEQ ID NO: 14.

### PglpF_SD10

In one or more exemplary embodiments, the heterologous regulatory element is PglpF_SD10 or variants thereof such as but not limited to SEQ ID NO: 15.

### PglpF_SD2

In one or more exemplary embodiments, the heterologous regulatory element is PglpF_SD2 or variants thereof such as but not limited to SEQ ID NO: 16.

### PglpF_SD3

In one or more exemplary embodiments, the heterologous regulatory element is PglpF_SD3 or variants thereof such as but not limited to SEQ ID NO: 17.

### PglpF_SD4

In one or more exemplary embodiments, the heterologous regulatory element is PglpF_SD4 or variants thereof such as but not limited to SEQ ID NO: 18.

### PglpF_SD5

In one or more exemplary embodiments, the heterologous regulatory element is PglpF_SD5 or variants thereof such as but not limited to SEQ ID NO: 19.

### PglpF_SD6

In one or more exemplary embodiments, the heterologous regulatory element is PglpF_SD6 or variants thereof such as but not limited to SEQ ID NO: 20.

### PglpF_SD7

In one or more exemplary embodiments, the heterologous regulatory element is PglpF_SD7 or variants thereof such as but not limited to SEQ ID NO: 21.

### PglpF_SD8

In one or more exemplary embodiments, the heterologous regulatory element is PglpF_SD8 or variants thereof such as but not limited to SEQ ID NO: 22.

### PglpF_SD9

In one or more exemplary embodiments, the heterologous regulatory element is PglpF_SD9 or variants thereof such as but not limited to SEQ ID NO: 23.

### PglpF_B28

In one or more exemplary embodiments, the heterologous regulatory element is PglpF_B28 or variants thereof such as but not limited to SEQ ID NO: 24.

### PglpF_B29

In one or more exemplary embodiments, the heterologous regulatory element is PglpF_B29 or variants thereof such as but not limited to SEQ ID NO: 25.

### Plac_16UTR

In one or more exemplary embodiments, the heterologous regulatory element is Plac_16UTR or variants thereof such as but not limited to SEQ ID NO: 26.

### Plac

In one or more exemplary embodiments, the heterologous regulatory element is Plac or variants thereof such as but not limited to SEQ ID NO: 27.

### PmglB_70UTR

In one or more exemplary embodiments, the heterologous regulatory element is PmglB_70UTR or variants thereof such as but not limited to SEQ ID NO: 9.

### PmglB_70UTR_SD4

In one or more exemplary embodiments, the heterologous regulatory element is PmglB_70UTR_SD4 or variants thereof such as but not limited to SEQ ID NO: 10.

### Episomal Element

The term “episomal element” refers to an extrachromosomal nucleic acid sequence, that can replicate autonomously or integrate into the genome of the genetically engineered cell. Thus, an episomal nucleic acid sequences may be a plasmid that can integrate into the chromosome of the genetically engineered cell, i.e. not all plasmids are episomal elements.

In one or more exemplary embodiments, episomal nucleic acid sequences may be a plasmid that is not integrated into the chromosome. In these embodiments, the episomal element refers to plasmid DNA sequences that carry an expression cassette of interest, with the cassette consisting of a promoter sequence, the coding sequence of the gene of interest and a terminator sequence.

In one or more exemplary embodiments, episomal nucleic acid sequences may be a plasmid with only a part of it being integrated into the chromosome. In these embodiments, the expression cassette resembles the one described above but it further comprises two DNA segments that are homologous to the DNA regions up- and downstream of the locus that the gene of interest is to be integrated.

### Activators

In one or more exemplary embodiment(s), the genetically engineered cell disclosed herein comprises an over-expressed gene product that enhances the expression of the gene(s) encoding the enzyme(s) required to facilitate the production of a human milk oligosaccharide(s) (HMOs), such as but not limited to LNFP-I, 2′-FL, LNT II and LNT.

In one or more exemplary embodiments, the cell of the present disclosure may comprise an over-expressed gene product that binds to the regulatory element of v) or regions upstream of the regulatory element of v) and enhances the expression of the proteins of i) to iii) or the colonic acid gene cluster of iv).

In one or more exemplary embodiments, the cell of the present disclosure may comprise an over-expressed gene product that binds to the regulatory element of v) or regions upstream of the regulatory element of v) and enhances the expression of the proteins of i) to iii) or the colonic acid gene cluster of iv), and wherein the heterologous α-1,2-fucosyltransferase protein is SEQ ID NO: 6.

### CRP

In one or more exemplary embodiments, said gene product is the cAMP DNA-binding transcriptional dual regulator CRP.

CRP belongs to the CRP-FNR superfamily of transcription factors. CRP regulates the expression of several of the E. coli genes, many of which are involved in catabolism of secondary carbon sources. Upon activation by cyclic-AMP, (cAMP) CRP binds directly to specific promoter sequences, the binding recruits the RNA polymerase through direct interaction, which in turn activates the transcription of the nucleic acid sequence following the promoter sequence leading to expression of the gene of interest. Thus, over-expression of CRP may lead to an enhanced expression of a gene/nucleic acid sequence of interest. Amongst other functions, CRP exerts its function on the PglpF promoters, where it contrary to the repressor GlpR, activates promoter sequences of the PglpF family. In this way, over-expression of CRP in the genetically engineered cell of the present disclosure, promotes expression of genes that are regulated by promoters of the PglpF family.

Thus, in one or more exemplary embodiments, the crp gene is over-expressed. The crp gene may encode a protein which is 100% identical to the amino acid sequence having the GenBank accession ID NP_417816 or a functional homologue thereof with is at least 70% identical, such as 80%, such as 90% such as 95% such as 98% identical to GenBank accession ID NP_417816.

Genetic engineering of GlpR and/or CRP as suggested in the present disclosure in 2′-FL producing strains is beneficial for the overall production of 2′-FL by these strains.

### Repressors

As shown in Example 5, the deletion of the glpR gene coding the DNA-binding transcriptional repressor GlpR is used as a genetic tool to obtain a specific target composition of a HMO mixture comprising up to four HMOs, including LNFP-I, 2′-FL, LNT II and LNT (in order of abundance).

In one or more exemplary embodiments, the method according to the present disclosure comprise a cell further comprising non-functional (or absent) gene product that binds to the regulatory element of v) or regions upstream of the regulatory element of v) and represses the expression of the proteins of i) to iii) or the colonic acid gene cluster of iv).

In one or more exemplary embodiments the method according to the present disclosure comprises a cell wherein a gene product that binds to the regulatory element of v) or regions upstream of the regulatory element of v) and represses the expression of any one of the proteins of i), ii) or iii) or the colonic acid gene cluster of iv), has been deleted or made non-functional.

In one or more exemplary preferred embodiments, the method according to the present disclosure comprise a cell further comprising a non-functional (or absent) gene product that binds to the regulatory element of v) or regions upstream of the regulatory element of v) and represses the expression of the proteins of i) to iii) or the colonic acid gene cluster of iv), and wherein the heterologous α-1,2-fucosyltransferase protein is SEQ ID NO: 6.

In one or more exemplary embodiments, said gene product is the DNA-binding transcriptional repressor GlpR.

### GlpR

GlpR belongs to the DeoR family of transcriptional regulators and acts as the repressor of the glycerol-3-phosphate regulon, which is organized in different operons. This regulator is part of the glpEGR operon, yet it can also be constitutively expressed as an independent (glpR) transcription unit. In addition, the operons regulated are induced when Escherichia coli is grown in the presence of inducer, glycerol, or glycerol-3-phosphate (G3P), and the absence of glucose. In the absence of inducer, this repressor binds in tandem to inverted repeat sequences that consist of 20-nucleic acid-long DNA target sites.

The term “non-functional or absent” in relation to the glpR gene refers to the inactivation of the glpR gene by complete or partial deletion of the corresponding nucleic acid sequence from the bacterial genome (e.g. SEQ ID NO: 48 or variants thereof encoding glpR capable of downregulating glpF derived promoters). The glpR gene can also be rendered non-functional by introducing mutations into the coding sequence which introduces stop codons, frameshifts or amino acid mutations that affects the DNA binding to the regulatory element. The glpR gene encodes the DNA-binding transcriptional repressor GlpR. In this way promoter sequences of the PglpF family are upregulated in the genetically engineered cell, due to deletion of the repressor gene that would otherwise downregulate the PglpF promoters.

In one or more exemplary embodiments, the glpR gene is deleted.

### Sugar Efflux Transporter

Over the past decade several new and efficient sugar efflux transporter proteins have been identified, each having specificity for different recombinantly produced HMOs and development of recombinant cells expressing said proteins are advantageous for high scale industrial HMO manufacturing. Sugar transport relates to the transport of a sugar, such as, but not limited to, an oligosaccharide.

For example, engineering futC-expressing cells by introducing one of several selected sugar efflux transporter proteins as well as increasing the expression levels of the colanic acid gene cluster proved to be two efficient genetic modifications that can markedly inverse the order of abundance of the first and second most abundant HMO of the final HMO blend from LNFP-I>2′-FL to 2′-FL>LNFP-I.

Thus, the genetically engineered cell(s) described herein, may also comprise a recombinant nucleic acid encoding a sugar efflux transporter. A sugar efflux transporter may for example enhance the level of an HMO in a method as described herein.

Influx and/or efflux transport of one/or more HMOs, from the cytoplasm or periplasm of a genetically engineered cell as described herein to the production medium and/or from the production medium to the cytoplasm or periplasm is disclosed.

A polypeptide, expressed in the genetically engineered cell as disclosed herein, capable of transporting one or more HMOs from the cytoplasm or periplasm to the production medium and/or from the production medium to the cytoplasm or periplasm of a genetically engineered cell, is a polypeptide capable of sugar transport.

Thus, in the present context, sugar transport can mean efflux and/or influx transport of sugar, such as, but not limited to, an HMO.

Thus, in one or more exemplary embodiments, the genetically engineered cell according to the method described herein further comprises a gene product that acts as a sugar efflux transporter. The gene product that acts as a sugar efflux transporter may be encoded by a recombinant nucleic acid sequence that is expressed in the genetically engineered cell. The recombinant nucleic acid sequence encoding a sugar efflux transporter, may be integrated into the genome of the genetically engineered cell. It may be plasmid borne, or it may be part of an episomal expression element.

MFS transporters Exemplary sugar efflux transporters are a subspecies of the Major Facilitator Superfamily proteins. The MFS transporters facilitate the transport of molecules, such as but not limited to sugars like oligosaccharides, across the cellular membranes.

By the term “Major Facilitator Superfamily (MFS)” is meant a large and exceptionally diverse family of the secondary active transporter class, which is responsible for transporting a range of different substrates, including sugars, drugs, hydrophobic molecules, peptides, organic ions, etc.

The term “MFS transporter” in the present context means, a protein that facilitates transport of an oligosaccharide, preferably, an HMO, through the cell membrane, preferably transport of an HMO/oligosaccharide synthesized by the genetically engineered cell as described herein from the cell cytosol to the cell medium. Additionally, or alternatively, the MFS transporter may also facilitate efflux of molecules that are not considered HMO or oligosaccharides, such as lactose, glucose, cell metabolites and/or toxins.

In Example 4, it is demonstrated how the introduction of selected heterologous genes coding sugar efflux transporter proteins in the genetic background of futC-expressing cells can markedly inverse the order of the abundance of the first and second most abundant HMO of the final HMO blend from LNFP-I>2′-FL to 2′-FL>LNFP-I. The only difference between these strains, as shown in Table 4, is the transporter gene that is integrated at a selected genomic locus of the host. Over-expression of such heterologous genes is believed to enhance 2′-FL and/or LNFP-I export from the cell interior to the extracellular environment, and thereby affect HMO production in multiple manners.

In one or more exemplary embodiments, the genetically engineered cell further comprises a recombinant nucleic acid encoding one or more sugar transport protein(s) as shown in Table 5.

In one or more exemplary embodiments, the sugar efflux transporter and/or MFS transport protein is selected from the group consisting of Bad, Nec, YberC, Fred, Vag and Marc.

In one or more presently preferred exemplary embodiments, the sugar efflux transporter is Nec or YberC.

### Bad

The MFS transporter protein identified herein as “Bad protein” or “Bad transporter” or “Bad”, interchangeably, has the amino acid sequence of SEQ ID NO: 28; The amino acid sequence identified herein as SEQ ID NO: 28 is an amino acid sequence which is 100% identical to the amino acid sequence having the GenBank accession ID WP_017489914.1.

In one or more exemplary embodiments, the sugar efflux transporter and/or MFS transport protein is Bad. In a further embodiment the sugar efflux transporter has the amino acid sequence of SEQ ID NO: 28 or is a functional homologue having an amino acid sequence which is at least 70% identical, such as at least 80% identical, such as at least 85% identical, such as at least 90% identical, such as at least 95% identical or such as at least 99% identical to any one of SEQ ID NO: 28.

### Nec

The MFS transporter protein having the amino acid sequence of SEQ ID NO: 29 is identified herein as “Nec protein” or “Nec transporter” or “Nec”, interchangeably; a nucleic acid sequence encoding Nec protein is identified herein as “Nec coding nucleic acid/DNA” or “nec gene” or “nec”; The amino acid sequence identified herein as SEQ ID NO: 29 is the amino acid sequence which is 100% identical to the amino acid sequence having the GenBank accession ID WP_092672081.1.

In one or more exemplary embodiments, the sugar efflux transporter and/or MFS transport protein is Nec. In a further embodiment the sugar efflux transporter has the amino acid sequence of SEQ ID NO: 29 or is a functional homologue having an amino acid sequence which is at least 70% identical, such as at least 80% identical, such as at least 85% identical, such as at least 90% identical, such as at least 95% identical or such as at least 99% identical to any one of SEQ ID NO: 29.

### YberC

The MFS transporter protein having the amino acid sequence of SEQ ID NO: 30 is identified herein as “YberC protein” or “YberC transporter” or “YberC”, interchangeably; a nucleic acid sequence encoding YberC protein is identified herein as “YberC coding nucleic acid/DNA” or “yberC gene” or “yberC”; The amino acid sequence identified herein as SEQ ID NO: 30 is the amino acid sequence which is 100% identical to the amino acid sequence having the GenBank accession ID EEQ08298.1.

In one or more exemplary embodiments, the sugar efflux transporter and/or MFS transport protein is YberC. In a further embodiment the sugar efflux transporter has the amino acid sequence of SEQ ID NO: 30 or is a functional homologue having an amino acid sequence which is at least 70% identical, such as at least 80% identical, such as at least 85% identical, such as at least 90% identical, such as at least 95% identical or such as at least 99% identical to any one of SEQ ID NO: 30.

### Fred

The MFS transporter protein having the amino acid sequence of SEQ ID NO: 31 is identified herein as “Fred protein” or “Fred transporter” or “Fred”, interchangeably; a nucleic acid sequence encoding freed protein is identified herein as “Fred coding nucleic acid/DNA” or “fred gene” or “fred”; The amino acid sequence identified herein as SEQ ID NO: 31 is the amino acid sequence which is 100% identical to the amino acid sequence having the GenBank accession ID WP_087817556.1.

In one or more exemplary embodiments, the sugar efflux transporter and/or MFS transport protein is Fred. In a further embodiment the sugar efflux transporter has the amino acid sequence of SEQ ID NO: 31 or is a functional homologue having an amino acid sequence which is at least 70% identical, such as at least 80% identical, such as at least 85% identical, such as at least 90% identical, such as at least 95% identical or such as at least 99% identical to any one of SEQ ID NO: 31.

### Vag

The MFS transporter protein having the amino acid sequence of SEQ ID NO: 32 is identified herein as “Vag protein” or “Vag transporter” or “Vag”, interchangeably; a nucleic acid sequence encoding Vag protein is identified herein as “Vag coding nucleic acid/DNA” or “vag gene” or “vag”; The amino acid sequence identified herein as SEQ ID NO: 32 is the amino acid sequence which is 100% identical to the amino acid sequence having the GenBank accession ID WP_048785139.1.

In one or more exemplary embodiments, the sugar efflux transporter and/or MFS transport protein is Vag. In a further embodiment the sugar efflux transporter has the amino acid sequence of SEQ ID NO: 32 or is a functional homologue having an amino acid sequence which is at least 70% identical, such as at least 80% identical, such as at least 85% identical, such as at least 90% identical, such as at least 95% identical or such as at least 99% identical to any one of SEQ ID NO: 32.

### Marc

The MFS transporter protein having the amino acid sequence of SEQ ID NO: 33 is identified herein as “Marc protein” or “Marc transporter” or “Marc”, interchangeably; a nucleic acid sequence encoding marc protein is identified herein as “Marc coding nucleic acid/DNA” or “marc gene” or “Marc”; The amino acid sequence identified herein as SEQ ID NO: 33 is the amino acid sequence which is 100% identical to the amino acid sequence having the GenBank accession ID WP_060448169.1.

In one or more exemplary embodiments, the sugar efflux transporter and/or MFS transport protein is Marc. In a further embodiment the sugar efflux transporter has the amino acid sequence of SEQ ID NO: 33 or is a functional homologue having an amino acid sequence which is at least 70% identical, such as at least 80% identical, such as at least 85% identical, such as at least 90% identical, such as at least 95% identical or such as at least 99% identical to any one of SEQ ID NO: 33.

In one or more exemplary embodiments, the sugar efflux transporter and/or MFS transport protein selected from the group consisting of Bad, Nec, YberC, Fred, Vag, and Marc, may be a functional homologue.

In one or more exemplary embodiments, a sugar efflux transporter functional homologue having an amino acid sequence which is at least 70% identical, such as at least 80% identical, such as at least 85% identical, such as at least 90% identical, such as at least 95% identical or such as at least 99% identical to any one of SEQ ID NOs: 28, 29, 30, 31, 32 or 33.

### Culturing

In the present context, culturing refers to the process by which cells are grown under controlled conditions, generally outside their natural environment, thus a method used to cultivate, propagate and grow a large number of cells.

### Cell Culture Medium

In the present context, a growth medium or culture medium is a liquid or gel designed to support the growth of microorganisms, cells, or small plants. The medium comprises an appropriate source of energy and may comprise compounds which regulate the cell cycle. The culture medium may be semi-defined, i.e. containing complex media compounds (e.g. yeast extract, soy peptone, casamino acids, etc.), or it may be chemically defined, without any complex compounds. The terms growth culture, culture medium and production medium are used interchangeably. Exemplary suitable media are provided in experimental examples.

In one or more exemplary embodiments, the culturing media is minimal media.

In one or more exemplary embodiments, the culturing media is supplemented with one or more energy and carbon sources selected form the group containing glycerol, sucrose, glucose and fructose.

In one or more exemplary embodiments, the culturing media is supplemented with one or more energy and carbon sources selected form the group containing glycerol, sucrose and glucose.

In one or more exemplary embodiments, the culturing media is supplemented with glycerol, sucrose and/or glucose.

In one or more exemplary embodiments, the culturing media is supplemented with glycerol and/or glucose.

In one or more exemplary embodiments, the culturing media is supplemented with sucrose and/or glucose.

In one or more exemplary embodiments, the culturing media is supplemented with glycerol and/or sucrose.

In one or more exemplary embodiments, the culturing media is supplemented only with sucrose.

In one or more exemplary embodiments, the culturing media contains sucrose as the sole carbon and energy source.

### Fermentation Temperature

Example 6 deals with the disclosure of how fermentation temperature can be advantageously used to modulate the composition of the HMO blend produced by strain MP21, and disclose that a particular strain, namely MP21, shows that the molar ratio of the two most abundant HMOs in this blend varies between e.g. 1.5:1 to 2:1 for LNFP-I:2′-FL, while the third most abundant HMO, namely LNT, can vary between approximately 5% and 1% by molar of the total HMO mixture in the same temperature interval.

In one or more exemplary embodiment(s), the fermentation temperature during the culturing of the genetically engineered cell in step (b) in the exemplary methods described above may be fixed to 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C.

The fermentation process described herein also include modulation of the fermentation temperature and/or lactose levels in the fermentation broth to achieve the HMO blend profile with a given strain derived from strain engineering, in a highly predictable manner. Hence, variation of fermentation temperature between for example 25 and 34° C., such as for example between 25 and 32° C. allowed to modulate the composition of a LNFP-I/2′-FL/LNT blend.

Thus, in one or more exemplary embodiments, the fermentation temperature during the culturing of the genetically engineered cell in step (b) in the exemplary methods described above is modulated. The modulation of the temperature during the culturing of the genetically engineered cell may be between 2° and 40° C., such as between 20-39° C., 20-38° C., 20-37° C., 20-36° C., 20-35° C., 20-34° C., 20-33° C., 20-32° C., 20-31° C., 20-30° C., 21-40° C., 22-40° C., 23-40° C., 24-40° C., 25-40° C., 26-40° C., 27-40° C., 28-40° C., 29-40° C., 30-40° C., 21-39° C., 22-38° C., 23-37° C., 24-36° C., 25-35° C., 25-34° C., 25-33° C., 25-32° C., 25-31° C., 25-30° C., 26-30° C., 27-30° C., 28-30° C., and 29-30° C.

As shown in Example 6, at 15 min after glucose feed start, the fermentation temperature setpoint was lowered from 33° C. to the respective setpoints under investigation, as shown in Tables 8 and 9. These temperature drops were conducted with a linear ramp over 3 hours. End-of-fermentation was at approximately 95-98 hours, when the target composition of the HMO mix and lactose had been reached. FIG. 6 depicts the development of the three major HMOs, namely LNFP-I, 2′-FL and LNT, produced by strain MP21 in fermentations at different production temperatures, and of the acceptor lactose. The LNFP-I/HMOL ratio increases to around 60% in all cases, 2′-FL and LNT show a highly temperature-dependent behaviour, where HMOL is the total sum of HMOs and lactose. Thus, at the end-of-fermentation, 2′-FL/HMOL ratio ranges from 15% to 35% while LNT/HMOL ratio ranges from 1% to 4%. Furthermore, while 2′-FL/HMOL increases proportionally with temperature in the range investigated (25-32° C.), LNT/HMOL behaves inversely proportional with temperature i.e., the lower the production temperature, the higher the LNT/HMOL ratio.

During process development studies for 2′-FL and 2′-FL/DFL products, we observed an impact of fermentation temperature and lactose levels (and the type of carbon source) on the DFL formation in 2′-FL producing strains.

In this case low temperature and low lactose levels favoured lower DFL formation, while here looking more closely into these parameters for LNFP-I producing strains. On the one hand, the situation in any LNFP-I producing strain is much more complex due to three different glycosyltransferases and sugar nucleotide pathways needed for the biosynthesis of LNFP-I compared to only one glycosyltransferase and sugar nucleotide pathway in 2′-FL strains. Yet, some of the observed effects was observed, arguably since LNFP-I strains and 2′-FL strains share the same alpha-1,2-fusosyltransferase.

Thus, in one or more exemplary embodiments, the ratio of 2′-FL/HMOL shows a proportional increase with temperature.

Thus, in one or more exemplary embodiments, the ratio of LNT/HMOL shows a proportional decrease with temperature.

### The Level of Lactose

As shown in Example 7, the level of lactose during fermentation showed a very big impact on the composition of a 4-HMO blend for one particular family of LNFP-I producing strains. Thus, in one or more exemplary embodiments, the level of lactose during the culturing of the genetically engineered cell in step (b) in the exemplary methods described above is modulated.

Low levels of lactose are when during the fermentation the lactose is below 20 g/L, preferably below 15 g/L, such as between 0.5 and 15 g/L, preferably below 10 g/L, such as between 1 and 10 g/L.

High levels of lactose is between 30-80 g/L for the first 40 h of the fermentation, following this the lactose will be allowed to deplete in order to reduce the lactose levels at end of fermentation and thereby reduce the downstream purification need to provide lactose free products if that is desired.

As depicted in FIG. 7, the two fermentation processes are otherwise identical with regard to medium composition, glucose feed profile and fermentation process parameters such as temperature, pH and dissolved oxygen. However, it was possible only by variation of the lactose levels from low (0.5-20 g/L) to high (30-80 g/L) to inverse the order of abundance of the second and third most abundant HMO quite remarkably from 2′-FL>>LNT to LNT>>2′-FL.

### HMO Product Profile at a High Lactose Level is LNFP-I>2′-FL>LNT>LNT-11

Thus, in one or more exemplary embodiments, the level of lactose during the culturing of the genetically engineered cell is modulated from low to high.

Thus, in one or more exemplary embodiments, the level of lactose during the culturing of the genetically engineered cell is modulated from low to high.

In one or more exemplary embodiments, a high level of lactose level relates to 30-80 g/L, such as but not limited to 30-40 g/L, 30-50 g/L, 30-60 g/L, 30-70 g/L, 40-50 g/L, 40-60 g/L, 40-70 g/L, 40-80 g/L, 50-60 g/L, 50-70 g/L, 50-80 g/L, 60-70 g/L, 60-80 g/L, 35-50 g/L, 35-60 g/L, 35-70 g/L, 35-75 g/L, 35-80 g/L, 45-55 g/L, 45-75 g/L, 55-65 g/L, 55-75 g/L, 55-80 g/L, 65-75 g/L, or 65-80 g/L.

Thus, in one or more exemplary embodiments, a low level of lactose level relates to 0-15 g/L, such as but not limited to 0-5 g/L, 0-7.5 g/L, 0-10 g/L, 0-12.5 g/L, 2.5-5 g/L, 2.5-7.5 g/L, 2.5-10 g/L, 2.5-12.5 g/L, 2.5-15 g/L, 5-7.5 g/L, 5-10 g/L, 5-12.5 g/L, 5-15 g/L, 7.5-10 g/L, 7.5-12.5 g/L, 7.5-15 g/L, 10-12.5 g/L, 10-15 g/L, or 12.5-15 g/L.

No other fermentation parameters besides temperature and lactose concentration were found to have such big impact on the HMO blend compositions for LNFP-I producing strains.

For many LNFP-I producing strains, derived by the above strain engineering strategies, two main fermentation parameters were surprisingly found to significantly impact the composition of the resulting HMO blend profiles in a predictable and unique manner, enabling us to deliver profiles with a pre-determined composition within narrow ranges for each of the HMOs contained in the blends.

Hence, variation of fermentation temperature between 25 and 32° C. allows us to modulate the composition of a LNFP-I/2′-FL/LNT blend. One study with a particular strain (see example 6), namely MP21, shows that the molar ratio of the two most abundant HMOs in this blend varies between 1.5:1 to 2:1 for LNFP-I:2′-FL, while the third most abundant HMO, namely LNT, can vary between approximately 5% and 1% by molar of the total HMO mixture in the same temperature interval.

### Sucrose Fermentation

In one or more exemplary embodiments, the genetically engineered cell may comprise a PTS-dependent sucrose utilization transport system and/or a recombinant nucleic acid sequence encoding a heterologous polypeptide capable of hydrolysing sucrose into fructose and glucose.

Such cells are capable of utilizing sucrose as carbon and energy source. For example, the culturing step according to step b) of the method(s) disclosed herein comprises a two-step sucrose feeding, with a second feeding phase by continuously adding to the culture an amount of sucrose that is less than that added continuously in a first feeding phase so as to slow the cell growth and increase the content of product produced in the high cell density culture.

The feeding rate of sucrose added continuously to the cell culture during the second feeding phase may be around 30-40% less than that of sucrose added continuously during the first feeding phase.

During both feeding phases, lactose can be added continuously, preferably with sucrose in the same feeding solution, or sequentially.

Optionally, the culturing further comprises a third feeding phase when considerable amount of unused acceptor remained after the second phase in the extracellular fraction.

Then the addition of sucrose is continued without adding the acceptor, preferably with around the same feeding rate set for the second feeding phase until consumption of the acceptor.

In one or more exemplary embodiments, the genetically engineered cell may comprise one or more heterologous nucleic acid sequence encoding one or more heterologous polypeptide(s) which enables utilization of sucrose as sole carbon and energy source of said genetically engineered cell.

In one or more exemplary embodiments, the genetically engineered cell comprises a PTS-dependent sucrose utilization system, further comprising the scrYA and scrBR operons.

In one or more exemplary embodiments, the polypeptide encoded by the scrYA operon are polypeptides with an amino acid sequence according to SEQ ID NO: 34 or SEQ ID NO: 35[scrY and scrA, respectively] or a functional homologue of any one of SEQ ID NO: 34 or SEQ ID NO: 35 [scrY and scrA, respectively], having an amino acid sequence which is at least 80% identical to any one of SEQ ID NO: 34 or SEQ ID NO: 35 [scrY and scrA, respectively].

In one or more exemplary embodiments the polypeptide encoded by the scrBR operon are polypeptides with an amino acid sequence according to SEQ ID NO: 36 or SEQ ID NO: 37 [scrB and scrR, respectively] or a functional homologue of any one of SEQ ID NO: 36 or SEQ ID NO:37 [scrB and scrR, respectively], having an amino acid sequence which is at least 80% identical to any one of SEQ ID NO: 36 or SEQ ID NO:37 [scrB and scrR, respectively]. Further details on the PTS-dependent sucrose utilization system are disclosed in WO2015/197082 (hereby incorporated by reference).

In one or more exemplary embodiments, the polypeptide capable of hydrolyzing sucrose into fructose and glucose is selected from the group consisting of SEQ ID NO: 38 or SEQ ID NO: 39 [SacC_Agal and Bff, respectively], or a functional homologue of any one of SEQ ID NO: 38 or SEQ ID NO: 39 [SacC_Agal and Bff, respectively], having an amino acid sequence which is at least 80% identical to any one of SEQ ID NO: 38 or SEQ ID NO: 39 [SacC_Agal and Bff, respectively].

### Harvesting

The term “harvesting” in the context relates to collecting the produced HMO(s) following the termination of fermentation. In one or more exemplary embodiments it may include collecting the HMO(s) included in both the biomass (i.e. the host cells) and cultivation media, i.e. before/without separation of the fermentation broth from the biomass. In other embodiments, the produced HMOs may be collected separately from the biomass and fermentation broth, i.e. after/following the separation of biomass from cultivation medium (i.e. fermentation broth).

The separation of cells from the medium can be carried out with any of the methods well known to the skilled person in the art, such as any suitable type of centrifugation or filtration. The separation of cells from the medium can follow immediately after harvesting the fermentation broth or be carried out at a later stage after storing the fermentation broth at appropriate conditions. Recovery of the produced HMO(s) from the remaining biomass (or total fermentation) include extraction thereof from the biomass (i.e., the production cells).

After recovery from fermentation, HMO(s) are available for further processing and purification.

### Genetically Engineered Cell

The genetically engineered cells described herein a heterologous β-1,3-N-acetyl-glucosaminyltransferase, β-1,3-galactosyltransferase and α-1,2-fucosyltransferase as described above and as shown in the Examples. In some embodiments the one or more of the heterologous β-1,3-N-acetyl-glucosaminyltransferase, β-1,3-galactosyltransferase and α-1,2-fucosyltransferase are overexpressed.

The present disclosure describes a genetically engineered cell for use in a method for producing oligosaccharides. Said genetically engineered cell has been genetically engineered to express
i. a heterologous β-1,3-N-acetyl-glucosaminyltransferase protein as shown in SEQ ID NO: 1 or SEQ ID NO: 2 or SEQ ID NO: 3, or a functional homologue thereof having an amino acid sequence which is at least 80% identical to SEQ ID NO: 1, 2 or 3; and
ii. a heterologous β-1,3-galactosyltransferase protein as shown in SEQ ID NO: 4 or SEQ ID NO: 5, or a functional homologue thereof having an amino acid sequence which is at least 80% identical to SEQ ID NO: 4 or 5; and
iii. a heterologous α-1,2-fucosyltransferase protein as shown in any one of SEQ ID NO: 6 or SEQ ID NO: 7 or SEQ ID NO: 49 or SEQ ID NO: 8 or a functional homologue thereof having an amino acid sequence which is at least 80% identical to any one of SEQ ID NO: 6, 7, 49 or 8, and
iv. the colanic acid gene cluster, and
v. a native or heterologous regulatory element for controlling the expression of the proteins of i), ii) and iii) or the colanic acid gene cluster in iv)

An aspect of the present invention relates to a genetically engineered cell comprising a recombinant nucleic acid sequence encoding
i. a heterologous β-1,3-N-acetyl-glucosaminyltransferase protein as shown in SEQ ID NO: 1 or 2 or 3, or a functional homologue thereof having an amino acid sequence which is at least 80% identical to SEQ ID NO: 1, 2 or 3; and
ii. a heterologous β-1,3-galactosyltransferase protein as shown in SEQ ID NO: 4 or 5, or a functional homologue thereof having an amino acid sequence which is at least 80% identical to SEQ ID NO: 4 or 5; and
iii. a heterologous α-1,2-fucosyltransferase protein as shown in any one of SEQ ID NO: 6 or SEQ ID NO: 7 or SEQ ID NO: 49 or a functional homologue thereof having an amino acid sequence which is at least 80% identical to any one of SEQ ID NO: 6, 7 or 49, and
iv. the colanic acid gene cluster, and
v. a native or heterologous regulatory or episomal element for controlling the expression of any of i)-iv).

Preferably, the genetically engineered cell further comprises a recombinant nucleic acid sequence encoding a sugar efflux transporter capable of exporting 2′FL and/or LNFP-I out of the cell.

The recombinant nucleic acid sequence encoding the sugar efflux transporter can be selected from the group consisting of:
a. a nucleic acid sequence encoding SEQ ID NO: 28 or a functional homologue thereof having an amino acid sequence which is at least 70% identical to SEQ ID NO: 28,
b. a nucleic acid sequence encoding SEQ ID NO: 29 or a functional homologue thereof having an amino acid sequence which is at least 70% identical to SEQ ID NO: 29,
c. a nucleic acid sequence encoding SEQ ID NO: 30 or a functional homologue thereof having an amino acid sequence which is at least 70% identical to SEQ ID NO: 30,
d. a nucleic acid sequence encoding SEQ ID NO: 31 or a functional homologue thereof having an amino acid sequence which is at least 70% identical to SEQ ID NO: 31,
   e. a nucleic acid sequence encoding SEQ ID NO: 32 or a functional homologue thereof having an amino acid sequence which is at least 70% identical to SEQ ID NO: 32, and
f. a nucleic acid sequence encoding SEQ ID NO: 33 or a functional homologue thereof having an amino acid sequence which is at least 70% identical to SEQ ID NO: 33.

Preferably, the genetically engineered cell overexpresses the colanic acid gene cluster by increasing the copy number and/or by choosing an appropriate regulatory element.

A “genetically modified” or genetically engineered” cell is used interchangeably herein and is understood as a cell whose genetic material has been altered by human intervention using a genetic engineering technique, such a technique is for example but not limited to transformation or transfection e.g., with a heterologous polynucleotide sequence, Crisper/Cas editing and/or random mutagenesis. In the present context, the terms α“genetically modified cell” and “a host cell” are used interchangeably.

In the present invention the “genetically modified cell” is preferably a host cell which has been transformed or transfected by an exogenous polynucleotide sequence.

In one or more exemplary embodiments, the cell is capable of producing one or more HMO(s) selected from the group consisting of 2′-FL, LNT-II, LNT, LNFP-I, and DFL.

In one or more exemplary embodiments, the genetically engineered cell is capable of producing one or more HMO(s) selected from the group consisting of 2′-FL, LNT-II, LNT and LNFP-I.

In one or more exemplary embodiments, the predominant HMO produced by the genetically engineered cell is LNFP-I and/or 2′-FL. In some embodiments the LNFP-I and/or 2′-FL fraction of the total amount of HMO produces is at least 70%, such as at least 80%, such as at least 85%, such as at least 95%, such as at least 98%.

In one or more exemplary embodiments, the HMO blend has a molar % of 2′-FL of between 25 to 70% and a molar % of LNFP-I of between 30% to 60% of the total HMO produced by the cell.

The genetically engineered cell may be any cell useful for HMO production including mammalian cell lines. Preferably, the host cell is a unicellular microorganism of eucaryotic or prokaryotic origin.

Appropriate microbial cells that may function as a host cell include yeast cells, bacterial cells, archaebacterial cells, algae cells, and fungal cells.

The genetically engineered cell (host cell) may be e.g., a bacterial or yeast cell. In one preferred embodiment, the genetically engineered cell is preferably a prokaryotic cell, such as a bacterial cell.

### Host Cells

Regarding the bacterial host cells, there are, in principle, no limitations, they may be eubacteria (gram-positive or gram-negative) or archaebacteria, as long as they allow genetic manipulation for insertion of a gene or regulatory element of interest and can be cultivated on a manufacturing scale. Preferably, the host cell has the property to allow cultivation to high cell densities.

Non-limiting examples of bacterial host cells that are suitable for recombinant industrial production of an HMO(s) according to the invention could be Erwinia herbicola (Pantoea agglomerans), Citrobacter freundii, Pantoea citrea, Pectobacterium carotovorum, or Xanthomonas campestris. Bacteria of the genus Bacillus may also be used, including Bacillus subtilis, Bacillus licheniformis, Bacillus coagulans, Bacillus thermophilus, Bacillus laterosporus, Bacillus megaterium, Bacillus mycoides, Bacillus pumilus, Bacillus lentus, Bacillus cereus, and Bacillus circulans. Similarly, bacteria of the genera Lactobacillus and Lactococcus may be engineered using the methods of this invention, including but not limited to Lactobacillus acidophilus, Lactobacillus salivarius, Lactobacillus plantarum, Lactobacillus helveticus, Lactobacillus delbrueckii, Lactobacillus rhamnosus, Lactobacillus bulgaricus, Lactobacillus crispatus, Lactobacillus gasseri, Lactobacillus casei, Lactobacillus reuteri, Lactobacillus jensenii, and Lactococcus lactis. Streptococcus thermophiles and Proprionibacterium freudenreichii are also suitable bacterial species for the invention described herein. Also included as part of this invention are strains, engineered as described here, from the genera Enterococcus (e.g., Enterococcus faecium and Enterococcus thermophiles), Bifidobacterium (e.g., Bifidobacterium longum, Bifidobacterium infantis, and Bifidobacterium bifidum), Sporolactobacillus spp., Micromomospora spp., Micrococcus spp., Rhodococcus spp., and Pseudomonas (e.g., Pseudomonas fluorescens and Pseudomonas aeruginosa).

Non-limiting examples of fungal host cells that are suitable for recombinant industrial production of an HMO(s) according to the invention could be yeast cells, such as Komagataella phaffii, Kluyveromyces lactis, Yarrowia lipolytica, Pichia pastoris, and Saccaromyces cerevisiae or filamentous fungi such as Aspargillus sp, Fusarium sp or Thricoderma sp, exemplary species are A. niger, A. nidulans, A. oryzae, F. solani, F. graminearum and T. reesei.

In one or more exemplary embodiments, the genetically engineered cell is S. cerevisiae or P pastoris.

In one or more exemplary embodiments, the genetically engineered cell is Pichia pastoris.

In one or more exemplary embodiments, the genetically engineered cell is S. cerevisiae.

In one or more exemplary embodiments, the genetically engineered cell is selected from the group consisting of E. coli, C. glutamicum, L. lactis, B. subtilis, S. lividans, P. pastoris, and S. cerevisiae.

In one or more exemplary embodiments, the genetically engineered cell is selected from the group consisting of B. subtilis, S. cerevisiae and Escherichia coli.

In one or more exemplary embodiments, the genetically engineered cell is B. subtilis.

In one or more exemplary embodiments, the genetically engineered cell is Escherichia coli.

In one or more exemplary embodiments, the invention relates to a genetically engineered cell, wherein the cell is derived from the E. coli K-12 or DE3 strain.

### Nucleic Acid Construct

The present disclosure describes the provision of a nucleic acid construct comprising recombinant nucleic acid sequence encoding one or more of the proteins selected from the group consisting of:
i. a heterologous β-1,3-N-acetyl-glucosaminyltransferase protein as shown in SEQ ID NO: 1 or SEQ ID NO: 2 or SEQ ID NO: 3, or a functional homologue thereof having an amino acid sequence which is at least 80% identical to SEQ ID NO: 1, 2 or 3; and
ii. a heterologous β-1,3-galactosyltransferase protein as shown in SEQ ID NO: 4 or SEQ ID NO: 5, or a functional homologue thereof having an amino acid sequence which is at least 80% identical to SEQ ID NO: 4 or 5; and
iii. a heterologous α-1,2-fucosyltransferase protein as shown in any one of SEQ ID NO: 6 or SEQ ID NO: 7 or SEQ ID NO: 8 or a functional homologue thereof having an amino acid sequence which is at least 80% identical to any one of SEQ ID NO: 6, 7 or 8, and
iv. the colanic acid gene cluster, and
   said nucleic acid construct further comprises at least one native or heterologous regulatory element for controlling the expression of the genes present in the nucleic acid construct, i.e. one or more of i)-iv).

In aspect of the present disclosure a nucleic acid construct comprises recombinant nucleic acid sequence encoding
i. a heterologous β-1,3-N-acetyl-glucosaminyltransferase protein as shown in SEQ ID NO: 1 or SEQ ID NO: 2 or SEQ ID NO: 3, or a functional homologue thereof having an amino acid sequence which is at least 80% identical to SEQ ID NO: 1, 2 or 3; and
ii. a heterologous β-1,3-galactosyltransferase protein as shown in SEQ ID NO: 4 or SEQ ID NO: 5, or a functional homologue thereof having an amino acid sequence which is at least 80% identical to SEQ ID NO: 4 or 5; and
iii. a heterologous α-1,2-fucosyltransferase protein as shown in SEQ ID NO: 6, 7 or 49 or a functional homologue thereof having an amino acid sequence which is at least 80% identical to any one of SEQ ID NO: 6, 7, or 49 and
iv. the colanic acid gene cluster, and
v. a native or heterologous regulatory element for controlling the expression of and of i)-iv)

The nucleic acid construct may comprise at least one regulatory element that facilitates the functional expression of the colanic acid gene cluster from its native genomic locus. Specifically, the colanic acid gene cluster may be overexpressed by increasing the copy number and/or by choosing an appropriate regulatory element.

In one embodiment the regulatory element is a recombinant promoter sequence derived from the promoter sequence of the lac operon or a g/p operon and one or more of the coding sequence of i) to iv) and the promoter sequence is operably linked.

An embodiment of the present invention is a nucleic acid construct comprising recombinant nucleic acid sequence encoding one or more of the protein(s) selected from the group consisting of:
i. a heterologous β-1,3-N-acetyl-glucosaminyltransferase protein as shown in SEQ ID NO: 1, or a functional homologue thereof having an amino acid sequence which is at least 80% identical to SEQ ID NO: 1,
ii. a heterologous β-1,3-galactosyltransferase protein as shown in SEQ ID NO: 4, or a functional homologue thereof having an amino acid sequence which is at least 80% identical to SEQ ID NO: 4, and
iii. a heterologous α-1,2-fucosyltransferase protein as shown in any one of SEQ ID NO: 8 or a functional homologue thereof having an amino acid sequence which is at least 80% identical to any one of SEQ ID NO: 8,
iv. the colanic acid gene cluster, and wherein the nucleic acid construct further comprises a native or heterologous regulatory or episomal element for controlling the expression of the genes encoding one or more of i)-iv), present in the nucleic acid construct.

The nucleic acid construct can be a recombinant nucleic acid sequence. By the term “recombinant nucleic acid sequence”, “recombinant gene/nucleic acid/DNA encoding” or “coding nucleic acid sequence” used interchangeably is meant an artificial nucleic acid sequence (i.e. produced in vitro using standard laboratory methods for making nucleic acid sequences) that comprises a set of consecutive, non-overlapping triplets (codons) which is transcribed into mRNA and translated into a protein when under the control of the appropriate control sequences, i.e. a promoter sequence.

The boundaries of the coding sequence are generally determined by a ribosome binding site located just upstream of the open reading frame at the 5′end of the mRNA, a transcriptional start codon (AUG, GUG or UUG), and a translational stop codon (UAA, UGA or UAG). A coding sequence can include, but is not limited to, genomic DNA, cDNA, synthetic, and recombinant nucleic acid sequences.

The term “nucleic acid” includes RNA, DNA and cDNA molecules. It is understood that, as a result of the degeneracy of the genetic code, a multitude of nucleic acid sequences encoding a given protein may be produced.

### A Recombinant Nucleic Acid Sequence

The recombinant nucleic sequence may be a coding DNA sequence e.g., a gene, or non-coding DNA sequence e.g., a regulatory DNA, such as a promoter sequence.

Accordingly, in one exemplified embodiment the invention relates to a nucleic acid construct comprising a coding nucleic sequence, i.e. recombinant DNA sequence of a gene of interest, e.g. a fucosyltransferase gene, and a non-coding regulatory DNA sequence, e.g. a promoter DNA sequence, e.g. a recombinant promoter sequence derived from the promoter sequence of lac operon or an g/p operon, or a promoter sequence derived from another genomic promoter DNA sequence, or a synthetic promoter sequence, wherein the coding and promoter sequences are operably linked.

The term “operably linked” refers to a functional relationship between two or more nucleic acid (e.g., DNA) segments. operably linked refers to the functional relationship of a transcriptional regulatory sequence to a transcribed sequence. For example, a promoter sequence is operably linked to a coding sequence if it stimulates or modulates the transcription of the coding sequence in an appropriate host cell or other expression system.

Generally, promoter sequences that are operably linked to a transcribed sequence are physically contiguous to the transcribed sequence, i.e., they are cis-acting.

In one exemplified embodiment, the nucleic acid construct of the invention may be a part of the vector DNA, in another embodiment the construct it is an expression cassette/cartridge that is integrated in the genome of a host cell.

Accordingly, the term “nucleic acid construct” means an artificially constructed segment of nucleic acid, in particular a DNA segment, which is intended to be ‘transplanted’ into a target cell, e.g. a bacterial cell, to modify expression of a gene of the genome or express a gene/coding DNA sequence which may be included in the construct.

Integration of the nucleic acid construct of interest comprised in the construct (expression cassette) into the bacterial genome can be achieved by conventional methods, e.g. by using linear cartridges that contain flanking sequences homologous to a specific site on the chromosome, as described for the attTn7-site (Waddell C. S. and Craig N. L., Genes Dev. (1988) February; 2(2):137-49.); methods for genomic integration of nucleic acid sequences in which recombination is mediated by the Red recombinase function of the phage λ or the RecE/RecT recombinase function of the Rac prophage (Murphy, J Bacteriol. (1998); 180(8):2063-7; Zhang et al., Nature Genetics (1998) 20: 123-128 Muyrers et al., EMBO Rep. (2000) 1(3): 239-243); methods based on Red/ET recombination (Wenzel et al., Chem Biol. (2005), 12(3):349-56.; Vetcher et al., Appl Environ Microbiol. (2005); 71(4):1829-35); or positive clones, i.e. clones that carry the expression cassette, can be selected e.g. by means of a marker gene, or loss or gain of gene function.

### Use of a Genetically Engineered

The disclosure also relates to any commercial use of the genetically engineered cell or the nucleic acid construct described herein.

Thus, in one or more exemplary embodiments, the genetically engineered cell or the nucleic acid construct according to the invention is used in the manufacturing of one or more HMOs. The one or more HMOs can be selected from the group consisting of 2′-FL, LNT-II, LNT, LNFP-I and DFL. In a presently preferred embodiment, the one or more HMOs is/are selected from the group consisting of 2′-FL, LNT-II, LNT, and LNFP-I.

In one or more exemplary embodiments, the genetically engineered cell and/or the nucleic acid construct is used in the manufacturing of more than one HMO(s), wherein the one or more HMOs is/are selected from the group consisting 2′-FL, LNT and LNFP-I.

In another exemplified embodiment, the genetically engineered cell and/or the nucleic acid construct according to the invention, is used in the manufacturing of more than one HMO(s), wherein the HMOs are 2′-FL and LNFP-I.

In another exemplified embodiment, the genetically engineered cell and/or the nucleic acid construct according to the invention, is used in the manufacturing of more than one HMO(s), wherein the predominant HMO blend has a molar % of 2′-FL of between 25 to 70% and a molar % of LNFP-I of between 30% to 60% of the total HMO produced.

### Manufacturing of HMOs

To produce one or more HMOs, the genetically engineered cell as described herein are cultivated according to the procedures known in the art in the presence of a suitable carbon source, e.g., glucose, glycerol, lactose, etc., and the produced HMO is harvested from the cultivation media and the microbial biomass formed during the cultivation process. Thereafter, the HMOs are purified according to the procedures known in the art, e.g., such as described in WO2015/188834, WO2017/182965 or WO2017/152918, and the purified HMOs are used as nutraceuticals, pharmaceuticals, or for any other purpose, e.g., for research.

Manufacturing of HMOs is typically accomplished by performing cultivation in larger volumes. The term “manufacturing” and “manufacturing scale” in the meaning of the invention defines a fermentation with a minimum volume of 5 L culture broth. Usually, a “manufacturing scale” process is defined by being capable of processing large volumes of a preparation containing the product of interest and yielding amounts of the protein of interest that meet, e.g., in the case of a therapeutic compound or composition, the demands for clinical trials as well as for market supply. In addition to the large volume, a manufacturing scale method, as opposed to simple lab scale methods like shake flask cultivation, is characterized by the use of the technical system of a bioreactor (fermenter) which is equipped with devices for agitation, aeration, nutrient feeding, monitoring and control of process parameters (pH, temperature, dissolved oxygen tension, back pressure, etc.). To a large extent, the behavior of an expression system in a lab scale method, such as shake flasks, benchtop bioreactors or the deep well format described in the examples of the disclosure, does allow to predict the behavior of that system in the complex environment of a bioreactor.

With regard to the suitable cell medium used in the fermentation process, there are no limitations. The culture medium may be semi-defined, i.e., containing complex media compounds (e.g., yeast extract, soy peptone, casamino acids, etc.), or it may be chemically defined, without any complex compounds. Where sucrose is used as the carbon and energy source, a minimal medium might be preferable.

### Manufactured Product

The term “manufactured product” according to the use of the genetically engineered cell or the nucleic acid construct refer to the one or more HMOs indented as the one or more product HMO. The various products are described above.

Advantageously, the methods disclosed herein provides both a decreased ratio of by-product to product and an increased overall yield of the product (and/or HMOs in total). This, less by-product formation in relation to product formation facilitates an elevated product production and increases efficiency of both the production and product recovery process, providing superior manufacturing procedure of HMOs.

The manufactured product may be a powder, a composition, a suspension, or a gel comprising one or more HMOs.

TABLE 1
Genotypes of the strains MP1, MP2, MP3 and MP4

| | | Heterologous Protein | SEQ ID NO in |
|---|---|---|---|
| Strain | | Sequence ID | the present |
| ID | Genotype | (Gen Bank) | application |
| MP1 | MDO, x1 GlcNAcT¹, x1 GalTK², x1 CA³, | WP_033911473.1 | SEQ ID NO: 1¹ |
| | x1 futC⁴ | BD182026.1 (modified)⁵ | SEQ ID NO: 4² |
| | | WP_080473865.1 | SEQ ID NO: 6⁴ |
| MP2 | MDO, x1 GlcNAcT¹, x1 GalTK², x1 CA³, | WP_033911473.1 | SEQ ID NO: 1¹ |
| | x1 smob⁴ | BD182026.1 (modified)⁵ | SEQ ID NO: 4² |
| | | WP_126455392.1 | SEQ ID NO: 8⁴ |
| MP3 | MDO, x1 GlcNAcT¹, x1 GalTK², x1 CA³, | WP_033911473.1 | SEQ ID NO: 1¹ |
| | x1 fucT54⁴ | BD182026.1 (modified)⁵ | SEQ ID NO: 4² |
| | | WP_013031010.1 | SEQ ID NO: 49⁴ |
| MP4 | MDO, x1 GlcNAcT¹, x1 GalTK², x1 CA³, | WP_033911473.1 | SEQ ID NO: 1¹ |
| | x1 mtun⁴ | BD182026.1 (modified)⁵ | SEQ ID NO: 4² |
| | | WP_031437198.1 | SEQ ID NO: 7⁴ |

¹GlcNAcT: lgtA gene coding for β-1,3-N-acetyl-glucosamintransferase (SEQ ID NO: 1)
²GalTK: gene coding for β-1,3-galactosyltransferase (SEQ ID NO: 4)
³CA: extra colanic acid gene cluster (gmd-wcaG-wcaH-wcaI-manC-manB, SEQ ID NO: 52) under the control of a PglpF promoter at a locus that is different than the native locus
⁴FutC: gene coding for α-1,2-fucosyltransferase (SEQ ID NO: 4)
⁵BD182026.1 is the nucleotide sequence encoding SEQ ID NO: 1 in U.S. Pat. No. 6,974,687
The SEQ ID's in the present application may be modified sequences of the public references, for the strain constructions the sequences in the SEQ ID NO's have been used.

TABLE 2
Genotypes of the strains MP5, MP6, MP7, MP8 and MP9

| | | Heterologous Protein | SEQ ID NO in |
|---|---|---|---|
| Strain | | Sequence ID | the present |
| ID | Genotype | (GenBank) | application |
| MP5 | MDO, x1 GlcNAcT¹, x1 GalTK², x1 CA³, x1 | WP_033911473.1 | SEQ ID NO: 1¹ |
| | Plac(CA)::PglpF-B28⁴, x1 futC⁵ | BD182026.1⁷ | SEQ ID NO: 4² |
| | | WP_080473865.1 | SEQ ID NO: 6⁵ |
| MP6 | MDO, x1 GlcNAcT¹, x1 GalTK², x1 | WP_033911473.1 | SEQ ID NO: 1¹ |
| | Plac(CA)::PglpF-B28⁴, x1 futC⁵ | BD182026.1⁷ | SEQ ID NO: 4² |
| | | WP_080473865.1 | SEQ ID NO: 6⁵ |
| MP7 | MDO, x1 GlcNAcT¹, x1 GalTK², x1 | WP_033911473.1 | SEQ ID NO: 1¹ |
| | Plac(CA)::PglpF-B29⁴, x1 futC⁵ | BD182026.1⁷ | SEQ ID NO: 4² |
| | | WP_080473865.1 | SEQ ID NO: 6⁵ |
| MP8 | MDO, x2 GlcNAcT¹, x1 GalTK², x1 CA³, x1 | WP_033911473.1 | SEQ ID NO: 1¹ |
| | smob⁶ | BD182026.1 (⁷ | SEQ ID NO: 4² |
| | | WP_126455392.1 | SEQ ID NO: 8⁶ |
| MP9 | MDO, x2 GlcNAcT¹, x1 GalTK², x1 | WP_033911473.1 | SEQ ID NO: 1¹ |
| | Plac(CA)::PglpF-B29⁴, x1 smob⁶ | BD182026.1⁷ | SEQ ID NO: 4² |
| | | WP_126455392.1 | SEQ ID NO: 8⁶ |

¹GlcNAcT: lgtA gene coding for β-1,3-N-acetyloglucosamine transferase (SEQ ID NO: 1)
²GalTK: gene coding for β-1,3-galactosyltransferase (SEQ ID NO: 4)
³CA: extra colanic acid gene cluster (gmd-wcaG-wcaH-wcaI-manC-manB SEQ ID NO: 52) under control of a PglpF promoter at a locus that is different than the native locus
⁴Plac(CA): promoter in front of the native CA gene cluster (i.e., Plac) of the MDO platform strain. In strains MP5 and MP6, the Plac is replaced by PglpF_B28, while in strains MP7 and MP9, it was replaced by PglpF_B29. MP5 expresses both the native CA cluster and an additional CA cluster whereas MP6, 7 and 9 just have a stronger promoter in front of the native CA cluster
⁵futC: gene coding for α-1,2-fucosyltransferase (SEQ ID NO: 6)
⁶smob: gene coding for α-1,2-fucosyltransferase (SEQ ID NO: 8)
⁷BD182026.1 is the nucleotide sequence encoding SEQ ID NO: 1 in U.S. Pat. No. 6,974,687
The SEQ ID's in the present application may be modified sequences of the public references, for the strain constructions the sequences in the SEQ ID NO's have been used

TABLE 3
Genotypes of the strains MP10 and MP11

| | | Heterologous Protein | SEQ ID NO in |
|---|---|---|---|
| Strain | | Sequence ID | the present |
| ID | Genotype | (GenBank) | application |
| MP10 | MDO, x1 GlcNAcT¹, x1 GalTK², x1 CA³, | WP_033911473.1 | SEQ ID NO: 1¹ |
| | x1 futC⁴ | BD182026.1⁵ | SEQ ID NO: 4² |
| | | WP_080473865.1 | SEQ ID NO: 6⁴ |
| MP11 | MDO, x2 GlcNAcT¹, x2 GalTK², x1 CA³, | WP_033911473.1 | SEQ ID NO: 1¹ |
| | x1 futC | BD182026.1⁵ | SEQ ID NO: 4² |
| | | WP_080473865.1 | SEQ ID NO: 6⁴ |

¹GlcNAcT: lgtA gene coding for β-1,3-N-acetyloglucosamine transferase (SEQ ID NO: 1)
²GalTK: gene coding for β-1,3-galactosyltransferase (SEQ ID NO: 4)
³CA: extra colanic acid gene cluster (gmd-wcaG-wcaH-wcaI-manC-manB SEQ ID NO: 52) under control of a PglpF promoter at a locus that is different than the native locus
⁴futC: gene coding for α-1,2-fucosyltransferase (SEQ ID NO: 6)
⁵BD182026.1 is the nucleotide sequence encoding SEQ ID NO: 1 in U.S. Pat. No. 6,974,687
The SEQ ID's in the present application may be modified sequences of the public references, for the strain constructions the sequences in the SEQ ID NO's have been used.

TABLE 4
Genotypes of the strains MP12, MP13, MP14, MP15, MP16, MP17 and MP18

| | | Heterologous Protein | SEQ ID NO in |
|---|---|---|---|
| Strain | | Sequence ID | the present |
| ID | Genotype | (GenBank) | application |
| MP12 | MDO, x2 GlcNAcT¹, x1 GalTK², x1 | WP_033911473.1 | SEQ ID NO: 1¹ |
| | Plac(CA)::PglpF-B28⁴, x1 futC⁵ | BD182026.1⁷ | SEQ ID NO: 4² |
| | | WP_080473865.1 | SEQ ID NO: 6⁵ |
| MP13 | MDO, x2 GlcNAcT¹, x1 GalTK², x1 | WP_033911473.1 | SEQ ID NO: 1¹ |
| | Plac(CA)::PglpF-B28⁴, x1 futC⁵, x1 nec⁶ | BD182026.1⁷ | SEQ ID NO: 4² |
| | | WP_080473865.1 | SEQ ID NO: 6⁵ |
| | | WP_092672081.1 | SEQ ID NO: 29⁶ |
| MP14 | MDO, x2 GlcNAcT¹, x1 GalTK², x1 | WP_033911473.1 | SEQ ID NO: 1¹ |
| | Plac(CA)::PglpF-B28⁴, x1 futC⁵, x1 vag⁶ | BD182026.1⁷ | SEQ ID NO: 4² |
| | | WP_080473865.1 | SEQ ID NO: 6⁵ |
| | | WP_048785139.1 | SEQ ID NO: 32⁶ |
| MP15 | MDO, x2 GlcNAcT¹, x1 GalTK², x1 | WP_033911473.1 | SEQ ID NO: 1¹ |
| | Plac(CA)::PglpF-B28⁴, x1 futC⁵, x1 yberC⁶ | BD182026.1⁷ | SEQ ID NO: 4² |
| | | WP_080473865.1 | SEQ ID NO: 6⁵ |
| | | EEQ08298.1 | SEQ ID NO: 30⁶ |
| MP16 | MDO, x2 GlcNAcT¹, x1 GalTK², x1 | WP_033911473.1 | SEQ ID NO: 1¹ |
| | Plac(CA)::PglpF-B28⁴, x1 futC⁵, x1 fred⁶ | BD182026.1⁷ | SEQ ID NO: 4² |
| | | WP_080473865.1 | SEQ ID NO: 6⁵ |
| | | WP_087817556.1 | SEQ ID NO: 31⁶ |
| MP17 | MDO, x2 GlcNAcT¹, x1 GalTK², x1 | WP_033911473.1 | SEQ ID NO: 1¹ |
| | Plac(CA)::PglpF-B28⁴, x1 futC⁵, x1 bad⁶ | BD182026.1⁷ | SEQ ID NO: 4² |
| | | WP_080473865.1 | SEQ ID NO: 6⁵ |
| | | WP_017489914.1 | SEQ ID NO: 28⁶ |
| MP18 | MDO, x2 GlcNAcT¹, x1 GalTK², x1 | WP_033911473.1 | SEQ ID NO: 1¹ |
| | Plac(CA)::PglpF-B28⁴, x1 futC⁵, x1 marc⁶ | BD182026.1⁷ | SEQ ID NO: 4² |
| | | WP_080473865.1 | SEQ ID NO: 6⁵ |
| | | WP_060448169.1 | SEQ ID NO: 33⁶ |

¹GlcNAcT: lgtA gene coding for β-1,3-N-acetyloglucosamine transferase (SEQ ID NO: 1)
²GalTK: gene coding for β-1,3-galactosyltransferase (SEQ ID NO: 4)
⁴Plac(CA): promoter in front of the native CA gene cluster (i.e., Plac) of the MDO platform strain. In all strains described in Table 4, the Plac promoter at the native CA locus was replaced by the PglpF_B28 promoter
⁵futC: gene coding for α-1,2-fucosyltransferase (SEQ ID NO: 6)
⁶gene coding for a heterologous sugar efflux transporter, MFS ((SEQ ID NO: 28-33)
⁷BD182026.1 is the nucleotide sequence encoding SEQ ID NO: 1 in U.S. Pat. No. 6,974,687
The SEQ ID's in the present application may be modified sequences of the public references, for the strain constructions the sequences in the SEQ ID NO's have been used.

TABLE 5
Transporter proteins of the Major Facilitator Superfamily
(MFS) that were introduced in LNFP-I production
strains MP12 to MP18 in table 4 above.

| MFS | | | |
|---|---|---|---|
| transporter | | | Promoter used |
| ID | Origin | GenBank ID | for expression |
| Bad | Rouxiella badensis | WP_017489914.1 | Plac |
| | | SEQ ID NO: 28 | SEQ ID NO: 27 |
| Nec | Rosenbergiella | WP_092672081.1 | Plac |
| | nectarea | SEQ ID NO: 29 | SEQ ID NO: 27 |
| YberC | Yersinia bercovieri | EEQ08298.1 | PglpF |
| | | SEQ ID NO: 30 | SEQ ID NO: 13 |
| Fred | Yersinia | WP_087817556.1 | PglpF |
| | frederiksenii | SEQ ID NO: 31 | SEQ ID NO: 13 |
| Vag | Pantoea vagans | WP_048785139.1 | PglpF |
| | | SEQ ID NO: 32 | SEQ ID NO: 13 |
| Marc | Serratia marcescens | WP_060448169.1 | Plac |
| | | SEQ ID NO: 33 | SEQ ID NO: 27 |

TABLE 6
Genotypes of the strains MP19 and MP20

| | | Heterologous | SEQ ID NO in |
|---|---|---|---|
| Strain | | Protein Sequence ID | the present |
| ID | Genotype | (GenBank) | application |
| MP19 | MDO, x2 GlcNAcT¹, x1 GalTK², x1 CA³, x1 | WP_033911473.1 | SEQ ID NO: 1¹ |
| | futC⁴ | BD182026.1⁵ | SEQ ID NO: 4² |
| | | WP_080473865.1 | SEQ ID NO: 6⁴ |
| MP20 | MDO, x2 GlcNAcT¹, x1 GalTK², x1 CA³, x1 | WP_033911473.1 | SEQ ID NO: 1¹ |
| | futC⁴, ΔglpR⁶ | BD182026.1⁵ | SEQ ID NO: 4² |
| | | WP_080473865.1 | SEQ ID NO: 6⁴ |
| | | | ΔSEQ ID NO: 48 |

¹GlcNAcT: lgtA gene coding for β-1,3-N-acetyloglucosamine transferase (SEQ ID NO: 1)
²GalTK: gene coding for β-1,3-galactosyltransferase (SEQ ID NO: 4)
³CA: extra colanic acid gene cluster (gmd-wcaG-wcaH-wcaI-manC-manB, SEQ ID NO: 52) under control of a PglpF promoter at a locus that is different than the native locus
⁴futC: gene coding for α-1,2-fucosyltransferase (SEQ ID NO: 6)
⁵BD182026.1 is the nucleotide sequence encoding SEQ ID NO: 1 in U.S. Pat. No. 6,974,687
The SEQ ID's in the present application may be modified sequences of the public references, for the strain constructions the sequences in the SEQ ID NO's have been used.
⁶GlpR: deletion of glp repressor gene (SEQ ID NO: 48)

TABLE 7
Genotype of the strain MP21

| | | Heterologous Protein | |
|---|---|---|---|
| Strain | | Sequence ID | |
| ID | Genotype | (GenBank) | Comment |
| MP21 | MDO, x1 GlcNAcT¹, x1 GalTK², x1 CA³, | WP_033911473.1 | This strain is |
| | x1 futC⁴ | BD182026.1⁵ | identical with |
| | | WP_080473865.1 | MP1 |

¹GlcNAcT: lgtA gene coding for β-1,3-N-acetyloglucosamine transferase (SEQ ID NO: 1)
²GalTK: gene coding for β-1,3-galactosyltransferase (SEQ ID NO: 4)
³CA: extra colanic acid gene cluster (gmd-wcaG-wcaH-wcaI-manC-manB, (SEQ ID NO: 52)) under control of a PglpF promoter at a locus that is different than the native locus
⁴futC: gene coding for α-1,2-fucosyltransferase (SEQ ID NO: 6)
⁵BD182026.1 is the nucleotide sequence encoding SEQ ID NO: 1 in U.S. Pat. No. 6,974,687
The SEQ ID's in the present application may be modified sequences of the public references, for the strain constructions the sequences in the SEQ ID NO's have been used.

TABLE 8
HMO blend composition in total broth sample
at end-of-fermentation (94.7 h-98.0 h).

| | | LNFP-I/ | 2′-FL/ | LNT/ | Lactose/ |
|---|---|---|---|---|---|
| Fermentation | Temp | HMOL | HMOL | HMOL | HMOL |
| batch | (° C.) | (%) | (%) | (%) | (%) |
| GDF20128 | 25 | 42.4 | 21.1 | 3.4 | 32.7 |
| GDF20153 | 26 | 44.6 | 34.1 | 3.2 | 16.8 |
| GDF20154 | 28 | 44.5 | 42.3 | 2.4 | 9.6 |
| GDF20155 | 30 | 46.4 | 48.2 | 1.2 | 2.7 |
| GDF20142 | 31 | 46.2 | 49.0 | 0.8 | 2.5 |
| GDF20156 | 32 | 45.5 | 49.4 | 1.0 | 2.9 |

HMOL = sum of HMOs incl. LNFP-I, 2′-FL, LNT, LNT II, DFL and lactose. All ratios in % by molar.

TABLE 9
HMO blend composition in total broth sample
at end-of-fermentation (94.7 h-98.0 h).

| Fermentation | Temp | LNFP-I/HMO* | 2′-FL/HMO* | LNT/HMO* |
|---|---|---|---|---|
| batch | (° C.) | (%) | (%) | (%) |
| GDF20128 | 25 | 63.0 | 31.3 | 5.1 |
| GDF20153 | 26 | 53.6 | 41.0 | 3.8 |
| GDF20154 | 28 | 49.3 | 46.7 | 2.7 |
| GDF20155 | 30 | 47.6 | 49.6 | 1.3 |
| GDF20142 | 31 | 47.4 | 50.3 | 0.8 |
| GDF20156 | 32 | 46.8 | 50.9 | 1.1 |

*HMO = sum of HMOs incl. LNFP-I, 2′-FL, LNT, LNT II, DFL without lactose, assuming that lactose can be selectively removed in DSP operations and/or fermentation process can be designed to end with minimal amount of residual lactose. All ratios in % by molar.

TABLE 10
Genotypes of the strains MP19 and MP22

| | | Heterologous | SEQ ID NO in |
|---|---|---|---|
| Strain | | Protein Sequence ID | the present |
| ID | Genotype | (GenBank) | application |
| MP19 | MDO, x2 GlcNAcT¹, x1 | WP_033911473.1 | SEQ ID NO: 1¹ |
| | GalTK², x1 CA³, | BD182026.1⁵ | SEQ ID NO: 4² |
| | x1 futC⁴ | WP_080473865.1 | SEQ ID NO: 6⁴ |
| MP22 | MDO, x2 GlcNAcT¹, x1 | WP_033911473.1 | SEQ ID NO: 1¹ |
| | GalTK², x1 CA³, | BD182026.1⁵ | SEQ ID NO: 4² |
| | x1 futC⁴, | WP_080473865.1 | SEQ ID NO: 6⁴ |

¹GlcNAcT: lgtA gene coding for β-1,3-N-acetyloglucosamine transferase (SEQ ID NO: 1)
²GalTK: gene coding for β-1,3-galactosyltransferase (SEQ ID NO: 4)
³CA: extra colanic acid gene cluster (gmd-wcaG-wcaH-wcaI-manC-manB, SEQ ID NO: 52) at a locus that is different than the native locus
⁴futC: gene coding for α-1,2-fucosyltransferase (SEQ ID NO: 6)
⁵BD182026.1 is the nucleotide sequence encoding SEQ ID NO: 1 in U.S. Pat. No. 6,974,687
The SEQ ID's in the present application may be modified sequences of the public references, for the strain constructions the sequences in the SEQ ID NO's have been used.
MP19 and MP22 differ in the genomic integration site for the second copy of GlcNAcT

TABLE 11
HMO blend composition in total broth sample at fermentation timepoint 68.7 h.

| | | Process | | | | | |
|---|---|---|---|---|---|---|---|
| Fermentation | | ID | LNFP- | | | LNT | |
| Batch ID | | (lactose | I/HMO | 2′-FL/HMO | LNT/HMO | II/HMO | |
| GDF17xxx | Strain | high/low*) | (%) | (%) | (%) | (%) | Remarks |
| 262 | MP19 | L2F20 | 59.9 | 4.0 | 27.2 | 9.0 | Foaming |
| | | (high) | | | | | at 68.7 h |
| 263 | MP19 | L2F21 | 70.0 | 24.7 | 4.0 | 1.3 | Foaming |
| | | (low) | | | | | at 93.0 h |
| 264 | MP22 | L2F20 | 67.5 | 5.9 | 20.7 | 5.9 | Foaming |
| | | (high) | | | | | at 68.7 h |
| 265 | MP22 | L2F21 | 65.7 | 29.5 | 3.4 | 1.1 | No |
| | | (low) | | | | | foaming |

*High/low lactose refers to a concentration range of 30-80 g/L for the high lactose process L2F20 and 0-15 g/L for the low lactose process L2F21, respectively.
HMO = sum of HMOs incl. LNFP-I, 2′-FL, LNT, LNT-II, and DFL.

TABLE 12
Genotype of the strains MP8, MP23, MP24, MP25

| | | Heterologous Protein | SEQ ID NO in |
|---|---|---|---|
| Strain | | Sequence ID | the present |
| ID | Genotype | (GenBank) | application |
| MP8 | MDO, x2 GlcNAcT¹, x1 GalTK², x1 CA³, x1 | WP_033911473.1 | SEQ ID NO: 1¹ |
| | smob⁴ | BD182026.1⁵ | SEQ ID NO: 4² |
| | | WP_126455392.1 | SEQ ID NO: 8⁴ |
| MP23 | MDO, x2 GlcNAcT¹, x1 GalTK², x1 CA³, x1 | WP_033911473.1 | SEQ ID NO: 1¹ |
| | smob⁴, x1 PglpF-nec⁶ | BD182026.1⁵ | SEQ ID NO: 4² |
| | | WP_126455392.1 | SEQ ID NO: 8⁴ |
| | | WP_092672081.1 | SEQ ID NO: 29⁶ |
| MP24 | MDO, x2 GlcNAcT¹, x1 GalTK², x1 CA³, x1 | WP_033911473.1 | SEQ ID NO: 1¹ |
| | smob⁴, x1 Plac-yberC⁶ | BD182026.1⁵ | SEQ ID NO: 4² |
| | | WP_126455392.1 | SEQ ID NO: 8⁴ |
| | | EEQ08298.1 | SEQ ID NO: 30⁶ |
| MP25 | MDO, x2 GlcNAcT¹, x1 GalTK², x1 CA³, x1 | WP_033911473.1 | SEQ ID NO: 1¹ |
| | smob⁴, x1 Plac-nec⁶ | BD182026.1⁵ | SEQ ID NO: 4² |
| | | WP_126455392.1 | SEQ ID NO: 8⁴ |
| | | WP_092672081.1 | SEQ ID NO: 29⁶ |

¹GlcNAcT: β-1,3-N-acetyloglucosamine transferase (SEQ ID NO: 1)
²GalTK: β-1,3-galactosyltransferase (SEQ ID NO: 4)
³CA: colanic acid gene cluster (gmd-wcaG-wcaH-wcaI-manC-manB, SEQ ID NO: 52) under control of a PglpF promoter at a locus that is different than the native locus
⁴gene coding for α-1,2-fucosyltransferase (SEQ ID NO: 6)
⁵BD182026.1 is the nucleotide sequence encoding SEQ ID NO: 1 in U.S. Pat. No. 6,974,687
⁶gene coding for a heterologous sugar efflux transporter, MFS (SEQ ID NO: 29 and 30)
The SEQ ID's in the present application may be modified sequences of the public references, for the strain constructions the sequences in the SEQ ID NO's have been used.

### General

It should be understood that any feature and/or aspect discussed above in connections with the compounds according to the disclosure apply by analogy to the methods described herein.

The terms Lacto-N-triose, LNT-II, LNT II, LNT2 and LNT 2, are used interchangeably.

The following figures and examples are provided below to illustrate the present disclosure. They are intended to be illustrative and are not to be construed as limiting in any way.

### BRIEF DESCRIPTION OF THE FIGURES

FIG. 1

Blends generated by strains expressing different α-1,2-fucosyltransferases: (a) HMO content of the blends (in % mM), (b) total HMO formation (in mM).

FIG. 2

The change in the HMO content (in mM %) of blends with varying expression levels of the colanic acid gene cluster in (a) smob-expressing cells and (b) futC-expressing cells, and the total HMO formation (in mM) in the corresponding blends (c).

FIG. 3

The effect of the copy number of the glycosyltransferases required for LNT synthesis on the HMO content (in % mM) of the final blend generated by strains (a) MP10 and (b) MP11

FIG. 4

The relative change in the total HMO, LNFP-I and 2′-FL content (in % mM) of the final blend generated by strains expressing different heterologous MFS transporters relative to the strain that does not have such a transporter.

FIG. 5

The effect of glpR +/−phenotype on the HMO content (in % mM) of the final blend generated by strains (a) MP19 and (b) MP20.

FIG. 6a-d

Time profiles of HMO blend composition in total broth samples throughout the whole fermentation runs at six different temperatures between 25° C. and 32° C. HMOL=sum of HMOs incl. LNFP-I, 2′-FL, LNT, LNT-II, DFL and lactose. DFL and LNT-II are below LNT, typically <1 g/L and not shown.

FIG. 7

Time profiles for the lactose monohydrate concentration in the fermentation broth throughout the four runs at either high lactose (process L2F20) or low lactose (process L2F21) condition using the two strains MP19 and MP22.

FIG. 8

Time profiles of the LNFP-I/HMO ratio in the fermentation broth throughout the four runs at either high lactose (process L2F20) or low lactose (process L2F21) condition using the two strains MP19 and MP22.

HMO=sum of HMOs incl. LNFP-I, 2′-FL, LNT, LNT-II and DFL. DFL is <0.3 g/L.

FIG. 9a-c

Time profiles of the ratios 2′-FL/HMO, LNT/HMO and LNT-II/HMO in the fermentation broth throughout the four runs at either high lactose (process L2F20) or low lactose (process L2F21) condition using the two strains MP19 and MP22. HMO=sum of HMOs incl. LNFP-I, 2′-FL, LNT, LNT-II and DFL. DFL is <0.3 g/L.

FIG. 10

Fraction of LNFP-I detected in the supernatant (in % of total LNFP-I) in cultures of smob-expressing cells that do not express a MFS transporter (strain MP8) and smob-expressing cells that bear a genomic copy of the nec (strains MP23 and MP25) or yberC (strain MP24) genes.

FIG. 11

Ratios of LNFP-I to 2′-FL in the final HMO blend for smob-expressing cells that do not express a MFS transporter (strain MP8) and cells that express a genomic copy of the nec (strains MP23 and MP25) or yberC (strain MP24) genes.

FIG. 12

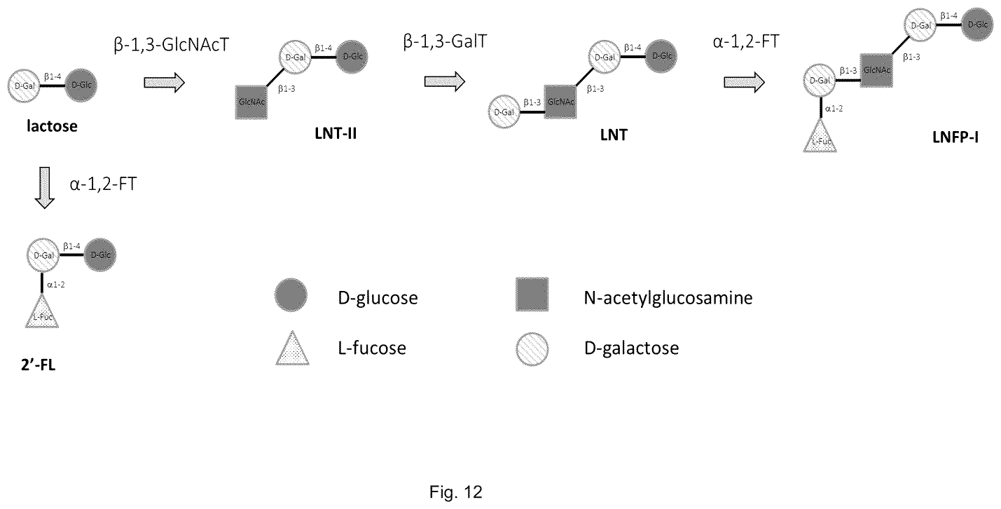

Pathways for producing LNFP-I and 2′-FL respectively from lactose. 2′FL is produced in a single step from lactose in the presence of the enzyme α-1,2-fucosyltransferase (α-1,2-ft) adding fucose to the lactose. Production of LNFP-I is a 3 step process where a β-1,3-N-acetyl-glucosaminyltransferase (β-1,3-GlcNacT) adds N-acetylglucosamine to lactose to form LNT-II to which a β-1,3-galactosyltransferase (β-1,3-GalT) adds galactose forming LNT on which an α-1,2-fucosyltransferase (α-1,2-ft) adds a fucose to form LNFP-I. As illustrated in example 1 different α-1,2-fucosyltransferase may have different substrate specificities, i.e. FutC seem to have higher specificity for lactose whereas smob seems to have higher specificity for LNT as substrate.

### SEQUENCE ID'S

The current application contains a sequence listing in text format and electronical format which is hereby incorporated by reference as are the sequences listed in the corrected sequence list in the priority application DK PA 2021 70247. Below is a summary of the sequences.
SEQUENCE ID NO 1 [lgtA PROTEIN—β-1,3-N-acetyl-glucosaminyltransferase]
SEQUENCE ID NO 2 [PmnagT PROTEIN—β-1,3-N-acetyl-glucosaminyltransferase]
SEQUENCE ID NO 3 [HDO466—β-1,3-N-acetyl-glucosaminyltransferase]
SEQUENCE ID NO 4 [galTK—β-1,3-galactosyltransferase]
SEQUENCE ID NO 5 [cvb3galT—β-1,3-galactosyltransferase]
SEQUENCE ID NO 6 [futC—α-1,2-fucosyltransferase]
SEQUENCE ID NO 7 [mtun—α-1,2-fucosyltransferase]
SEQUENCE ID NO 8 [smob—α-1,2-fucosyltransferase]
SEQ ID NO: 9 [pmglB_70UTR]
SEQ ID NO: 10 [pmglB_70UTR_SD4]
SEQ ID NO: 11 [Pscr]
SEQ ID NO: 12 [PgatY_70UTR]
SEQ ID NO: 13 [PglpF]
SEQ ID NO: 14 [PglpF_SD1]
SEQ ID NO: 15 [PglpF_SD10]
SEQ ID NO: 16 [PglpF_SD2]
SEQ ID NO: 17 [PglpF_SD3]
SEQ ID NO: 18 [PglpF_SD4]
SEQ ID NO: 19 [PglpF_SD5]
SEQ ID NO: 20 [PglpF_SD6]
SEQ ID NO: 21 [PglpF_SD7]
SEQ ID NO: 22 [PglpF_SD8]
SEQ ID NO: 23 [PglpF_SD9]
SEQ ID NO: 24 [PglpF_B28]
SEQ ID NO: 25 [PglpF_B29]
SEQ ID NO: 26 [Plac_16UTR]
SEQ ID NO: 27 [Plac]
SEQ ID NO: 28 [Bad]
SEQ ID NO: 29 [Nec]
SEQ ID NO: 30 [YberC]
SEQ ID NO: 31 [Fred]
SEQ ID NO: 32 [Vag]
SEQ ID NO: 33 [Marc]
SEQ ID NO: 34 [ScrY]
SEQ ID NO: 35 [ScrA]
SEQ ID NO: 36 [ScrB CAA47974.1]
SEQ ID NO: 37 [ScrR]
SEQ ID NO: 38 [SacC_Agal]
SEQ ID NO: 39 [Bff]
SEQ ID NO: 40 [Igta gene]
SEQ ID NO: 41 [PmnagT gene]
SEQ ID NO: 42 [HDO466 gene]
SEQ ID NO: 43 [galtk gene]
SEQ ID NO: 44 [cvb3galT]
SEQ ID NO: 45 [futC gene encoding α-1,2-fucosyltransferase]
SEQ ID NO: 46 [mtun gene encoding α-1,2-fucosyltransferase]
SEQ ID NO: 47 [smob gene encoding α-1,2-fucosyltransferase]
SEQ ID NO: 48 [DNA-binding transcriptional repressor GlpR]
SEQ ID NO: 49 [fucT54 α-1,2-fucosyltransferase]
SEQ ID NO: 50 Oligo 048, galK.for
SEQ ID NO: 51 Oligo 049, galK.rev
SEQ ID NO: 52 CA gene cluster

### Items

1. A method for the production of a human milk oligosaccharide (HMO) blend with LNFP-I and 2′-FL as the predominant HMO's, the method comprising the steps of:
a) providing a genetically engineered cell capable of producing HMOs, wherein said cell
   i) comprises a heterologous β-1,3-N-acetyl-glucosaminyltransferase protein as shown in SEQ ID NO: 1 or 2 or 3, or a functional homologue thereof having an amino acid sequence which is at least 80% identical to SEQ ID NO: 1 or 2 or 3; and
   ii) comprises a heterologous β-1,3-galactosyltransferase protein as shown in SEQ ID NO: 4 or 5, or a functional homologue thereof having an amino acid sequence which is at least 80% identical to SEQ ID NO: 4 or 5; and
   iii) comprises a heterologous α-1,2-fucosyltransferase protein as shown in any one of SEQ ID NO: 6 or 7 or 49 or 8 or a functional homologue thereof having an amino acid sequence which is at least 80% identical to any one of SEQ ID NO: 6, 7 or 49 or 8, and
   iv) expresses functionally the colanic acid gene cluster, and
   v) comprises a native or heterologous regulatory element for controlling the expression of and of i)-iv)
b) culturing the cell according to (a) in a suitable cell culture medium to express said proteins and to produce an HMO blend; and
c) harvesting the human milk oligosaccharide (HMO) blend produced in step (b).

2. The method according to item 1, wherein the overexpression of the protein(s) in i), ii) and iii) is provided by increasing the copy number of the genes coding said protein(s) and/or by choosing an appropriate regulatory element for the colonic acid gene cluster in iv).

3. The method according to any of the preceding items wherein the expression of the colanic acid gene cluster in iv) is modulated by swapping the native promoter with a promoter of interest, and/or increasing the copy number of the colanic acid genes coding said protein(s) by expressing the gene cluster from another genomic locus, or episomally expressing the colanic acid gene cluster.

4. The method according to any of the preceding items, wherein the heterologous regulatory element is selected from the group of promoters consisting of SEQ ID NO: 13 (PglpF), SEQ ID NO: 12 (PgatY_70UTR), SEQ ID NO: 27 (Plac), SEQ ID NO: 9 (PmglB_70UTR), SEQ ID NO: 11 (Pscr), or a variant thereof.

5. The method according to any of the preceding items, wherein the heterologous regulatory element is selected from the group consisting of PBAD, Pxyl, PsacB, PxylA, PrpR, PnitA, PT7, Ptac, PL, PR, PnisA, Pb, Pscr, PgatY_70UTR, PglpF, PglpF_SD1, PglpF_SD10, PglpF_SD2, PglpF_SD3, PglpF_SD4, PglpF_SD5, PglpF_SD6, PglpF_SD7, PglpF_SD8, PglpF_SD9, PglpF_B28, Plac_16UTR, Plac, PmglB_70UTR and PmglB_70UTR_SD4.

6. The method according to any of the preceding items, wherein the heterologous regulatory element is selected from the group consisting of PglpF, Pscr, Plac, PglpF_B29, and PglpF_B28.

7. The method according to any one of the preceding items, wherein the expression of i) and ii) is obtained from a single copy and/or the regulatory element for expression of i) and ii) has low or intermediate strength.

8. The method according to item 7, wherein the regulatory element is selected from the group consisting of PglpF_SD9 (SEQ ID NO: 23), PglpF_SD7 (SEQ ID NO: 21), PglpF_SD6 (SEQ ID NO: 20), PglpF_B28 (SEQ ID NO: 24), PglpF_B29 (SEQ ID NO: 25), Pscr (SEQ ID NO: 11 and Plac (SEQ ID NO: 27).

9. The method according to any of the preceding claims, wherein the regulatory element in is a strong regulatory element.

10. The method according to item 9, wherein the regulatory element is selected from the group consisting of PglpF (SEQ ID NO: 13) PglpF_SD10 (SEQ ID NO: 15), PglpF_SD8 (SEQ ID NO: 22), PglpF_SD5 (SEQ ID NO: 19), PglpF_SD4 (SEQ ID NO: 18), PgatY_70UTR (SEQ ID NO: 12), PmglB_70UTR (SEQ ID NO: 9) and PmglB_70UTR_SD4 (SEQ ID NO: 9).

11. The method according to any one of the preceding items, wherein the heterologous β-1,3-N-acetyl-glucosaminyltransferase protein is SEQ ID NO: 1, or a functional homologue thereof having an amino acid sequence which is at least 80% identical to SEQ ID NO: 1.

12. The method according to anyone of the preceding items, wherein the heterologous β-1,3-galactosyltransferase protein is SEQ ID NO: 4 or a functional homologue thereof having an amino acid sequence which is at least 80% identical to SEQ ID NO: 4.

13. The method according to any of the preceding items, wherein the expression of the colonic acid gene cluster in iv) is modulated by swapping the native promoter with a promoter of interest, and/or increasing the copy number of the colanic acid genes coding said protein(s) by expressing the gene cluster from another genomic locus, or episomally expressing the colonic acid gene cluster, and wherein the heterologous α-1,2-fucosyltransferase protein is SEQ ID NO: 6 or 8 or a functional homologue thereof having an amino acid sequence which is at least 80% identical to any one of SEQ ID NO: 6 or 8.

14. The method according to any of the preceding items, wherein the overexpression of the protein(s) in
i) and ii) is provided by the simultaneous increase in the copy number of the genes coding said protein(s), and wherein the heterologous α-1,2-fucosyltransferase protein is SEQ ID NO: 6 or a functional homologue thereof having an amino acid sequence which is at least 80% identical to any one of SEQ ID NO: 6.

15. The method according to any of the preceding items, wherein the HMO blend has molar % of 2′-FL between 25% to 70% and LNFP-I between 30% to 60%.

16. The method according to item 13 or 14, wherein the molar % of 2′-FL in the produced blend of HMOs is between 30% to 70%, such as between 40% and 55%, such as between 50% and 70%.

17. The method according to item 13 or 14, wherein the molar % of LNFP-I is between 30% to 60%, such as between 40% and 55%, such as between 30% and 45%.

18. The method according to any of items 1 to 12, wherein the heterologous α-1,2-fucosyltransferase protein is SEQ ID NO: 7 or 49 or a functional homologue thereof having an amino acid sequence which is at least 80% identical to any one of SEQ ID NO: 7 or 49.

19. The method according to item 18, wherein the molar % of 2′-FL in the produced blend of HMOs is between 40% to 60%, such as between 45% and 55%.

20. The method according to item 18, wherein the molar % of LNFP-I is between 40% to 60%, such as between 40% and 55.

21. The method according to any of the preceding items, wherein a gene product that binds to v) or regions upstream of v) and represses the expression of any one of i) to iv) has been deleted or made non-functional in the cell, and wherein the heterologous α-1,2-fucosyltransferase protein is SEQ ID NO: 6.

22. The method according to item 21, wherein said gene product is the DNA-binding transcriptional repressor GlpR.

23. The method according to any of the preceding items, wherein the cell further comprises a gene product that acts as a sugar efflux transporter.

24. The method according to item 23, wherein the sugar efflux transporter is selected from the group consisting of Bad, Nec, YberC, Fred, Vag, and Marc.

25. The method according to item 24, wherein the sugar efflux transporter is selected from the group consisting an amino acid sequence selected from
i) SEQ ID NO: 28 or a functional homologue thereof having an amino acid sequence which is at least 70% identical, such as at least 80% identical, such as at least 85% identical, such as at least 90% identical, such as at least 95% identical or such as at least 99% identical to SEQ ID NO: 28,
ii) SEQ ID NO: 29 or a functional homologue thereof having an amino acid sequence which is at least 70% identical, such as at least 80% identical, such as at least 85% identical, such as at least 90% identical, such as at least 95% identical or such as at least 99% identical to SEQ ID NO: 29,
iii) SEQ ID NO: 30 or a functional homologue thereof having an amino acid sequence which is at least 70% identical, such as at least 80% identical, such as at least 85% identical, such as at least 90% identical, such as at least 95% identical or such as at least 99% identical to SEQ ID NO: 30,
iv) SEQ ID NO: 31 or a functional homologue thereof having an amino acid sequence which is at least 70% identical, such as at least 80% identical, such as at least 85% identical, such as at least 90% identical, such as at least 95% identical or such as at least 99% identical to SEQ ID NO: 31,
v) SEQ ID NO: 32 or a functional homologue thereof having an amino acid sequence which is at least 70% identical, such as at least 80% identical, such as at least 85% identical, such as at least 90% identical, such as at least 95% identical or such as at least 99% identical to SEQ ID NO: 32, and
vi) SEQ ID NO: 33 or a functional homologue thereof having an amino acid sequence which is at least 70% identical, such as at least 80% identical, such as at least 85% identical, such as at least 90% identical, such as at least 95% identical or such as at least 99% identical to SEQ ID NO: 33.

26. The method according to item 24 or 25, wherein the sugar efflux transporter is preferably Nec or YberC.

27. The method according to item 26, wherein the heterologous α-1,2-fucosyltransferase protein is SEQ ID NO: 6 [futC] or SEQ ID NO: 7 [mtun] or SEQ ID NO: 49 [FucT54] or a functional homologue thereof having an amino acid sequence which is at least 80% identical to any one of SEQ ID NO: 6, 7 or 48.

28. The method according to item 27, wherein the molar % of 2′-FL in the produced blend of HMOs is between 30% to 70%, such as between 40% and 55%, such as between 50% and 60%.

29. The method according to any of the preceding items, wherein the fermentation temperature during the culturing of the genetically engineered cell in step (b) is modulated.

30. The method according to item 29, wherein the 2′-FL/HMOL ratio shows a proportional increase with increasing fermentation temperature, where the fermentation temperature is between 25 and 34° C., preferably between 30 to 32° C. between.

31. The method according to item 29, wherein the fermentation temperature during the culturing of the genetically engineered cell in step (b) is between 25 and 34° C., and wherein the molar % of 2′-FL is between 15% and 40% of the produced blend of HMOs.

32. The method according to item 30 wherein the heterologous α-1,2-fucosyltransferase protein is SEQ ID NO: 6 [futC].

33. The method according to item 29 or 30, wherein LNT/HMOL ratio shows a proportional decrease with increasing fermentation temperature.

34. The method according to any of the preceding items, wherein the level of lactose during the culturing of the genetically engineered cell in step (b) is modulated.

35. The method according to item 34, wherein the HMO product profile at a low lactose level, such as below 20 g/L, preferably below 15 g/L, such as between 0.5 and 15 g/L, preferably below 10 g/L, such as between 1 and 10 g/L, is LNFP-I>2′-FL>LNT>LNT-II.

36. The method according to item 34, wherein the HMO product profile at a high lactose level, such as between 30-80 g/L for the first 40 h of the fermentation, is LNFP-I>LNT>LNT-II>2′-FL.

37. The method according to any of the preceding items, wherein said genetically engineered cell comprises a one or more heterologous nucleic acid sequence(s) encoding one or more heterologous polypeptide(s) which enables utilization of sucrose as sole carbon and energy source of said genetically engineered cell.

38. The method according to item 37, wherein the sucrose utilization system is a polypeptide capable of hydrolysing sucrose into glucose and fructose, selected from the group consisting of SEQ ID NOs: 38 [SacC_Agal, glycoside hydrolase family 32 protein, WP_103853210.19Q ID NO; and 39 [Bff, beta-fructofuranosidase protein, BAD18121.1], or a functional homologue of any one of SEQ ID NOs: 11 and 12, having an amino acid sequence which is at least 80% identical, to any one of SEQ ID NOs: 38 or 39

39. A genetically engineered cell comprising a recombinant nucleic acid sequence encoding
i) a heterologous β-1,3-N-acetyl-glucosaminyltransferase protein as shown in SEQ ID NO: 1 or 2 or 3, or a functional homologue thereof having an amino acid sequence which is at least 80% identical to SEQ ID NO: 1-3; and
ii) a heterologous β-1,3-galactosyltransferase protein as shown in SEQ ID NO: 4 or 5, or a functional homologue thereof having an amino acid sequence which is at least 80% identical to SEQ ID NO: 4-5; and
iii) a heterologous α-1,2-fucosyltransferase protein as shown in any one of SEQ ID NO: 6 or 7 or 8 or a functional homologue thereof having an amino acid sequence which is at least 80% identical to any one of SEQ ID NO: 6-8, and
iv) the colanic acid gene cluster, and
v) a native or heterologous regulatory or episomal element for controlling the expression of and of i)-iv).

40. The genetically engineered cell according to item 39, which further comprises a recombinant nucleic acid sequence encoding a sugar efflux transporter capable of exporting 2′FL and/or LNFP-I out of the cell.

41. The genetically engineered cell according to item 40, wherein the recombinant nucleic acid sequence encoding a sugar efflux transporter is selected from the group consisting of:
i) a nucleic acid sequence encoding SEQ ID NO: 28 or a functional homologue thereof having an amino acid sequence which is at least 70% identical, such as at least 80% identical, such as at least 85% identical, such as at least 90% identical, such as at least 95% identical or such as at least 99% identical to SEQ ID NO: 28,
ii) a nucleic acid sequence encoding SEQ ID NO: 29 or a functional homologue thereof having an amino acid sequence which is at least 70% identical, such as at least 80% identical, such as at least 85% identical, such as at least 90% identical, such as at least 95% identical or such as at least 99% identical to SEQ ID NO: 29,
iii) a nucleic acid sequence encoding SEQ ID NO: 30 or a functional homologue thereof having an amino acid sequence which is at least 70% identical, such as at least 80% identical, such as at least 85% identical, such as at least 90% identical, such as at least 95% identical or such as at least 99% identical to SEQ ID NO: 30,
iv) a nucleic acid sequence encoding SEQ ID NO: 31 or a functional homologue thereof having an amino acid sequence which is at least 70% identical, such as at least 80% identical, such as at least 85% identical, such as at least 90% identical, such as at least 95% identical or such as at least 99% identical to SEQ ID NO: 31,
v) a nucleic acid sequence encoding SEQ ID NO: 32 or a functional homologue thereof having an amino acid sequence which is at least 70% identical, such as at least 80% identical, such as at least 85% identical, such as at least 90% identical, such as at least 95% identical or such as at least 99% identical to SEQ ID NO: 32, and
vi) a nucleic acid sequence encoding SEQ ID NO: 33 or a functional homologue thereof having an amino acid sequence which is at least 70% identical, such as at least 80% identical, such as at least 85% identical, such as at least 90% identical, such as at least 95% identical or such as at least 99% identical to SEQ ID NO: 33.

42. The genetically engineered cell according to item 39 to 41, wherein the colanic acid gene cluster is overexpressed by increasing the copy number and/or by choosing an appropriate regulatory element.

43. The genetically engineered cell according to item 39 or 40, wherein the heterologous β-1,3-N-acetyl-glucosaminyltransferase protein is SEQ ID NO: 1, or a functional homologue thereof having an amino acid sequence which is at least 80% identical to SEQ ID NO: 1.

44. The genetically engineered cell according to item 39 to 43, wherein the heterologous β-1,3-galactosyltransferase protein is SEQ ID NO: 4 or a functional homologue thereof having an amino acid sequence which is at least 80% identical to SEQ ID NO: 4.

45. The genetically engineered cell according to item 39 to 44, wherein the heterologous α-1,2-fucosyltransferase protein as shown in SEQ ID NO: 6, or a functional homologue thereof having an amino acid sequence which is at least 80% identical to SEQ ID NO: 6.

46. The genetically engineered cell according to item 39 to 44, wherein the heterologous α-1,2-fucosyltransferase protein as shown in SEQ ID NO: 7 or 49, or a functional homologue thereof having an amino acid sequence which is at least 80% identical to SEQ ID NO: 7 or 49.

47. A nucleic acid construct comprising a recombinant nucleic acid sequence encoding one or more of the proteins selected from the group consisting of:
i) a heterologous β-1,3-N-acetyl-glucosaminyltransferase protein as shown in SEQ ID NO: 1 or 2 or 3, or a functional homologue thereof having an amino acid sequence which is at least 80% identical to SEQ ID NO: 1-3; and
ii) a heterologous β-1,3-galactosyltransferase protein as shown in SEQ ID NO: 4 or 5, or a functional homologue thereof having an amino acid sequence which is at least 80% identical to SEQ ID NO: 4-5; and
iii) a heterologous α-1,2-fucosyltransferase protein as shown in any one of SEQ ID NO: 6 or 7 or 8 or a functional homologue thereof having an amino acid sequence which is at least 80% identical to any one of SEQ ID NO: 6-8, and
iv) the colanic acid gene cluster, and wherein said nucleic acid construct further comprises at least one native or heterologous regulatory element or episomal element for controlling the expression of the genes present in the nucleic acid construct, i.e., one or more of and of i)-iv).

48. The nucleic acid construct according to item 47, wherein the regulatory element is a recombinant promoter sequence derived from the promoter sequence of the lac operon or a g/p operon and one or more of the coding sequence of i) to iv) and the promoter sequence is operably linked.

49. Use of a genetically engineered cell according to any one of items 40 to 41, or the nucleic acid construct according to item 47 or 48, in the production of an HMO blend.

50. The use according to item 49, wherein the HMO blend comprises HMOs selected from the group consisting of 2′-FL, LNT-II, LNT, LNFP-I and DFL.

51. The use according to items 49 or 50, wherein the HMO blend predominantly contains 2′-FL and LNFP-I.

52. The use according to any of items 49-51, wherein the HMO blend has molar % of LNFP-I between 30 to 60% and 2′-FL between 30-70% of the total HMO.

### EXAMPLES

### Materials and Methods

### Construction of Strains

As background strains for the strains used in the examples below the bacterial strain MDO, was used. MDO is constructed from Escherichia coli K-12 DH1. The E. coli K-12 DH1 genotype is: F⁻, λ-, gyrA96, recA1, relA1, endA1, thi-1, hsdR17, supE44. In addition to the E. coli K-12 DH1 genotype MDO has the following modifications: lacZ: deletion of 1.5 kbp, lacA: deletion of 0.5 kbp, nanKETA: deletion of 3.3 kbp, me/A: deletion of 0.9 kbp, wcaJ: deletion of 0.5 kbp, mdoH: deletion of 0.5 kbp, and insertion of Plac promoter upstream of the gmd gene.

Methods of inserting gene(s) of interest into the genome of E. coli is well known to the person skilled in the art. The genotypes of the strains used in the present application are shown in tables 1, 2, 3, 4, 6, 7, 10 and 12.

As an example, the genomic insertion of an MFS transporter by replacing the GalK loci is described. An expression cassette containing a promoter linked to the fred gene and to a T1 transcriptional terminator sequence in the chromosomal DNA of E. coli K-12 DH1 MDO was performed by Gene Gorging essentially as described by Herring et al. (Herring et al 2003. Gene 311:153-163). Briefly, the donor plasmid and the helper plasmid were co-transformed into MDO and selected on LB plates containing 0.2% glucose, ampicillin (100 μg/mL) or kanamycin (50 mg/mL) and chloramphenicol (20 μg/mL). A single colony was inoculated in 1 mL LB containing chloramphenicol (20 μg/mL) and 10 μL of 20% L-arabinose and incubated at 37° C. with shaking for 7 to 8 hours. For integration in the galK loci of E. coli cells were then plated on M9-DOG plates and incubated at 37° C. for 48 hours. Single colonies formed on MM-DOG plates were re-streaked on LB plates containing 0.2% glucose and incubated for 24 hours at 37° C. Colonies that appeared white on MacConkey-galactose agar plates and were sensitive for both ampicillin and chloramphenicol were expected to have lost the donor and the helper plasmid and contain an insertion in the galK loci. Insertions in the galK site was identified by colony PCR using primers 048 (SEQ ID NO: 50) and 049 (SEQ ID NO: 51) and the inserted DNA was verified by sequencing (Eurofins Genomics, Germany).

Insertion of genetic cassettes at other loci in the E. coli chromosomal DNA can be done in a similar way using gene gorging (see for example Herring and Blattner 2004 J. Bacteriol. 186: 2673-81 and Warminget al 2005 Nucleic Acids Res. 33(4): e36) with different selection marker genes and different screening methods.

### Deep Well Assay Protocol for Strain Characterization

The strains disclosed in the present example were screened in 96 deep well plates using a 4-day protocol. During the first 24 hours, precultures were grown to high densities and subsequently transferred to a medium that allowed induction of gene expression and product formation. More specifically, during day 1, fresh precultures were prepared using a basal minimal medium supplemented with magnesium sulphate, thiamine and glucose. The precultures were incubated for 24 hours at 34° C. and 1000 rpm shaking and then further transferred to a new basal minimal medium (BMM, pH 7,5) in order to start the main culture. The new BMM was supplemented with magnesium sulphate, thiamine, a bolus of 20% glucose solution (50 ul per 100 mL) and a bolus of 10% lactose solution (5 ml per 100 ml). Moreover, 50% sucrose solution was provided as carbon source, accompanied by the addition of sucrose hydrolase (invertase), so that glucose was released at a rate suitable for C-limited growth. The main cultures were incubated for 72 hours at 28° C. and 1000 rpm shaking.

For the analysis of total broth, the 96-well plates were boiled at 100° C., subsequently centrifuged, and finally the supernatants were analysed by HPLC. For supernatant samples, the initial centrifugation of microtiter plates was followed by the removal of 0.1 mL supernatant for direct analysis by HPLC. For pellet samples, the cells were initially washed, then dissolved in deionized water and centrifuged.

Following centrifugation, the pellets were analysed for HMO content in the cell interior after resuspension, boiling, centrifugation and analysis of the final supernatant.

The millimolar content (mM) of the detected HMOs in each sample was calculated based on the reported analytical data, and the mM percentage (%) of each HMO in the final blend was calculated in relation to the total HMO (mM) concentration in the blend in order to easily compare the quantitative differences in the HMO blends generated by each strain.

### Example 1—Generation of Variations of HMO Blends of LNFP-I, 2′-FL and LNT by Testing Different Wild-Type α-1,2-fucosyltransferases

Description of the genotype of strains MP1, MP2, MP3 and MP4 tested in deep well assays Based on the previously reported platform strain (“MDO”), the modifications summarised in Table 1, were made to obtain the LNFP-I producing strains MP1, MP2, MP3 and MP4 used in this study, all being fully chromosomal strains. The strains can produce the tetrasaccharide HMO, LNT and fucosylate LNT further to obtain the pentasaccharide HMO, LNFP-I. The fucosyltransferase enzymes that can be used for this reaction are numerous, but in the framework of the present disclosure, we selected a small subset of α-1,2-fucosyltransferases from different bacterial species for testing their ability to fucosylate lactose and LNT. The selected enzymes include FutC from Helicobacter pylori (GenBank ID: WP_080473865.1, but with two additional amino acids (LG) at the C-terminus, SEQ ID NO: 6), Smob from Sulfuriflexus mobilis (GenBank ID: WP_126455392.1, SEQ ID NO: 8), FucT54 from Sideroxydans lithotrophicus ES-11 (GenBank ID: WP_013031010.1, SEQ ID NO: 49) and mtun from Methylobacter tundripaludum (GenBank ID: WP_031437198.1, SEQ ID NO: 7).

In the present Example, it was demonstrated how the choice of an α-1,2-fucosyltransferase can be used as a genetic tool to obtain defined and diverse target compositions of HMO blends consisting of almost exclusively LNFP-I and 2′-FL. This disclosure demonstrates how the choice of an α-1,2-fucosyltransferase can be advantageously used to modulate the composition of the HMO blend produced by strains MP1, MP2, MP3 and MP4. The only difference between these strains, as shown in Table 1, is the α-1,2-fucosyltransferase gene that was introduced at a selected genomic locus of the host to drive the in vivo decoration of lactose and LNT for the synthesis of HMOs, or precursor sugars thereof. The different enzymes showed different specificity for lactose and LNT, which was clearly reflected on the relative abundance of LNFP-I and 2′-FL in the acquired final HMO blend.

### Results of the Deep Well Assays

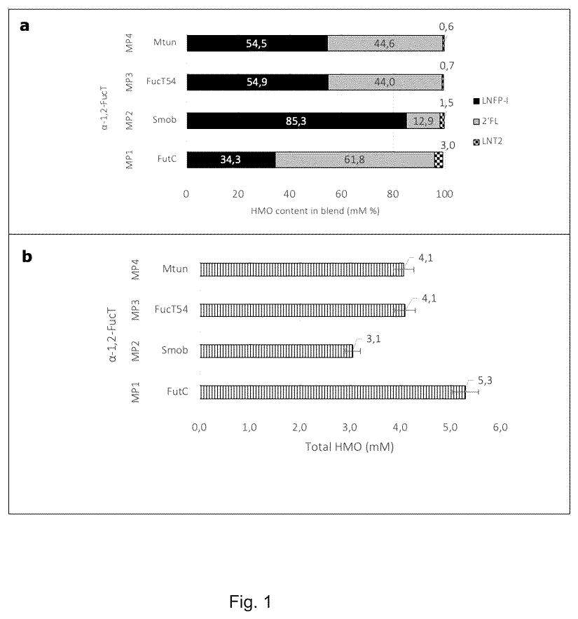

Strains were characterized in deep well assays as described in the “Materials and method section”. As shown in FIG. 1a, the choice of the α-1,2-fucosyltransferase resulted in marked differences in the HMO abundance of the final blend. Compared to the other enzymes, FutC seems to have higher specificity for lactose than LNT, a fact that led to HMO blends, where 2′-FL was the most abundant HMO. On the contrary, Smob resulted in blends, where LNFP-I was the predominant HMO and 2′-FL was the second most abundant sugar, while LNT-II was formed only in limited amounts. Smob can fucosylate LNT with an extraordinary specificity, while its specificity for lactose is seemingly very low. Finally, FucT54 and mtun led to HMO blends with almost equimolar concentrations of LNFP-I and 2′-FL, which is indicative of their almost even specificity for LNT and lactose (FIG. 1a).

The total HMO concentration in the blends generated by the strains under discussion differed significantly. There exists a strong correlation between high 2′-FL and total HMO concentrations, while higher LNFP-I content in the final HMO blend is accompanied by lower total HMO concentrations (FIG. 1b).

In conclusion, the choice of the α-1,2-fucosyltransferase, which can be introduced in the genetic background of a LNT production strain to produce LNFP-I, can significantly change the relevant HMO abundance of the mixture in such a manner that the final blend consists of either almost exclusively LNFP-I (MP2, Smob), or largely 2′-FL (MP1, FutC), or LNFP-I and 2′-FL in a ratio close to 1:1 (MP3, FucT54 and MP4, mtun).

### Example 2—Generation of Variations of HMO Blends of LNFP-I, 2′-FL and LNT by Increasing the Expression Levels of the Colanic Acid Gene Cluster

Description of the genotype of strains MP5, MP6, MP7, MP8 and MP9 tested in deep well assays Based on the previously reported platform strain (“MDO”), the modifications summarised in the table 2, were made to obtain the LNFP-I producing strains MP5, MP6, MP7, MP8 and MP9 used in this study, all being fully chromosomal strains. The strains can produce the tetrasaccharide HMO, LNT and fucosylate LNT further to obtain the pentasaccharide HMO, LNFP-I. The fucosyltransferase enzymes used for this reaction were either Smob (MP8 and MP9) or FutC (MP5, MP6 and MP7). As discussed in the Example 1, the two enzymes show different specificities for lactose and LNT and yield LNFP-I or 2′-FL as the predominant HMO in the final blend. Likewise, other HMOs, such as LNT and LNT-II are present in such HMO blends, but at lower concentrations.

In the present Example, it was demonstrated how increasing the expression level of the colanic acid gene cluster can be used as a genetic tool to either increase the LNFP-I to 2′-FL ratio in smob-expressing cells, or inverse the order of abundance of the first and second most abundant HMO of the final HMO blend from LNFP-I>2′-FL to 2′-FL>LNFP-I in futC-expressing cells. This disclosure demonstrates that the fine tuning of expression of the colanic acid genes can be advantageously used to modulate the composition of the HMO blends generated by smob- and futC-expressing cells.

### Results of the Deep Well Assays

Strains were characterized in deep well assays as described in the “Materials and method section”.

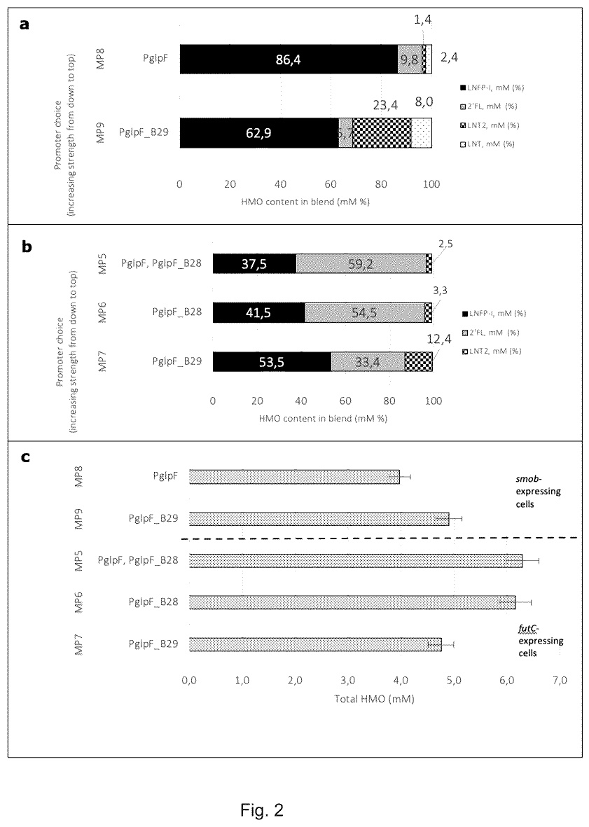

As shown in FIG. 2a-c, a single genetic modification, namely an increase in the expression levels of the colanic acid gene cluster, resulted in a drastic change in the HMO blend acquired by smob- and futC-expressing cells. Specifically, increasing the strength of the promoter that drives the expression of the colanic acid genes in smob-expressing cells (i.e., replacing the PglpF_B29 with the PglpF promoter) can increase the LNFP-I fraction of the HMO blend by almost 25% and on the same time reduce the LNT II+LNT fractions by almost 30% (FIG. 2a). Thus, this single simple promoter swapping in front of the native colanic acid locus can lead to the generation of a HMO blend that is largely consisting of LNFP-I (85%), with 2′-FL being the second most abundant HMO and making up only 10% of the total HMO content of the blend. However, it is noteworthy that the total HMO concentration in the blends generated by smob-expressing cells that differentially express the colanic acid gene cluster differs markedly. Specifically, the higher-level expression of the colanic acid gene cluster is seemingly linked to less total HMO formation (FIG. 2c): the strain MP9 produces almost 5 mM of HMOs, while the strain MP8 produces just 4 mM of total sugar.

Furthermore, as shown in FIG. 2b, the gradual increase in the promoter strength and/or the copy number of the colanic acid gene cluster (i.e., PglpF_B29<PglpF_B28<PglpF+PglpF_B28) in futC-expressing cells can gradually increase the 2′-FL fraction of the HMO blend by up to 25% and on the same time gradually reduce the LNFP-I fraction of the blend by a similar percentage (FIG. 2b). In detail, a simple promoter swapping in front of the native colanic acid locus and the introduction of a second copy of this gene cluster at a different genomic locus (i.e., PglpF_B29→PglpF_B28+PglpF) can invert the order of the abundance of the first and second most abundant HMO of the final HMO blend from LNFP-I>2′-FL to 2′-FL>LNFP-I in futC-expressing cells. It is noteworthy that the genetic modification under discussion, i.e., PglpF_B29→PglpF_B28+PglpF, has also largely improved the total HMO concentration in the final blend. Specifically, the genetic modification in the strain MP5 (PglpF+PglpF_B28) results in an almost 35% higher total HMO content compared to the one in the strain MP7 (PglpF_B29).

In conclusion, increasing the expression levels of the colanic acid gene cluster is a great genetic tool that enables the increase of the LNFP-I to 2′-FL ratio in smob-expressing cells, or the inversion of the order of the abundance of the first and second most abundant HMO of the final HMO blend from LNFP-I>2′-FL to 2′-FL>LNFP-I in futC-expressing cells.

Example 3—Generation of variations of HMO blends of LNFP-I, 2′-FL and LNT by increasing the copy number of glycosyltransferases involved in LNT formation Description of the genotype of strains MP10 and MP11 tested in deep well assays Based on the previously reported platform strain (“MDO”), the modifications summarised in table 3, were made to obtain the LNFP-I producing strains MP10 and MP11 used in this study, both being fully chromosomal strains. The strains can produce the tetrasaccharide HMO, LNT and fucosylate LNT further to obtain the pentasaccharide HMO, LNFP-I. The fucosyltransferase enzyme used for this reaction was the FutC enzyme from Helicobacter pylori (GenBank ID: WP_080473865.1, but with two additional amino acids (LG) at the C-terminus, SEQ ID NO: 6).

In the present Example, it was demonstrated how the fine tuning of the copy number of the genes coding the glycosyltransferases being involved in LNT biosynthesis can be used as a genetic tool to invert the order of the abundance of the first and second most abundant HMO of the acquired blend from 2′-FL>LNFP-I to LNFP-I>2′-FL in futC-expressing cells. This disclosure demonstrates how the simultaneous change in the copy number of the IgtA (coding a β-1,3-N-acetyl-glucosaminyltransferase) and galTK (coding a β-1,3-galactosyltransferase) genes in futC-expressing cells can be advantageously used as a means to modulate the composition of the HMO blend produced by strains MP10 and MP11. As shown in Table 3, the only difference between the two strains is the presence of an additional IgtA and galTK copy in the genetic background of the strain MP11 compared to the background of the strain MP10. The additional copies of the IgtA and galTK genes in MP11 are believed to boost LNT production and thereby increase LNFP-I and/or overall HMO production.

### Results of the Deep Well Assays

Strains were characterized in deep well as described in the “Materials and method section”.

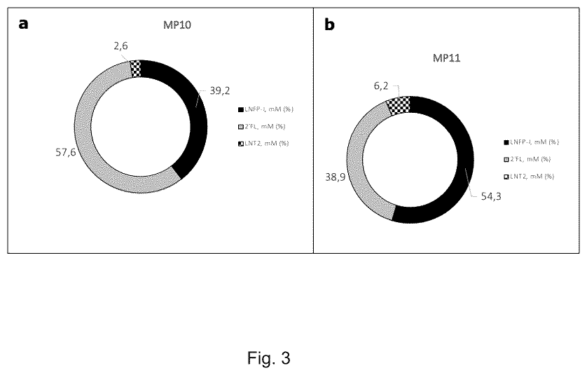

As shown in FIG. 3, the simultaneous increase in IgtA and galTK copy numbers in the LNFP-I production strain MP10 to generate the strain MP11, resulted in a marked change in the acquired HMO blend. Specifically, this modification was able to invert the abundancy of the two major HMOs of the final blends, i.e., LNFP-I and 2′-FL. The strain with a lower gene copy number, MP10, generated a blend consisting of 58% 2′-FL and 40% LNFP-I, while the strain with a higher gene copy number, MP11, provided a blend with the inverse HMO profile of 40% 2′-FL and 55% LNFP-I. The total HMO concentration in the final HMO blends generated by these strains differed significantly, with the strain MP10 (one copy of each gene) generating a blend with a 15% higher total HMO content compared to the one generated by the strain MP11 (two copies of each gene). Thus, the genetic modification under discussion leads to changes in both the LNFP-I and 2′-FL fractions in the final HMO blend, but also to the obtained total HMO concentrations (data not shown).

In conclusion, the simultaneous increase in the copy number of the genes coding the glycosyltransferases involved in LNT biosynthesis is an effective tool to invert the order of the abundance of the first and second most abundant HMO of the acquired HMO blend from 2′-FL>LNFP-I to LNFP-I>2′-FL in futC-expressing cells.

### Example 4—Generation of Variations of HMO Blends of LNFP-I, 2′-FL and LNT by Introducing Sugar Efflux Transporters of the Major Facilitator Superfamily (MFS)

### Description of the Genotype of Strains MP12, MP13, MP14, MP15, MP16, MP17 and MP18 Tested in Deep Well Assays

Based on the previously reported platform strain (“MDO”), the modifications summarised in Table 4, were made to obtain the LNFP-I producing strains MP12, MP13, MP14, MP15, MP16, MP17 and MP18 used in this study, all being fully chromosomal strains. The strains can produce the tetrasaccharide HMO, LNT and fucosylate LNT further to obtain the pentasaccharide HMO, LNFP-I. The fucosyltransferase enzyme used for this reaction was the FutC enzyme from Helicobacter pylori (GenBank ID: WP_080473865.1, but with two additional amino acids (LG) at the C-terminus, SEQ ID NO: 6). Notably, other HMOs, such as LNT and LNT II were present in the final HMO blends generated by the above-mentioned strains, but only at minimal concentrations.

In the present Example, it was demonstrated how the introduction of selected heterologous genes encoding sugar efflux transporter proteins (Table 5) in the genetic background of futC-expressing cells can markedly inverse the order of the abundance of the first and second most abundant HMO of the final HMO blend from LNFP-I>2′-FL to 2′-FL>LNFP-I. In this regard, the genetic tool presented here is equivalent to the one described in Example 2 above, where the increase in the expression of the colanic acid gene cluster in futC-expressing cells was shown to also invert the profile of the two most abundant HMOs in the blend from LNFP-I>2′-FL to 2′-FL>LNFP-I. The disclosure discussed here demonstrates how the introduction of genes coding the selected heterologous sugar efflux transporter proteins can be advantageously used to modulate the composition of the HMO blend produced by the strains MP12, MP13, MP14, MP15, MP16, MP17 and MP18. The only difference between these strains, as shown in Table 4, is the transporter gene that is integrated at a selected genomic locus of the host. Over-expression of such heterologous genes is believed to enhance 2′-FL and/or LNFP-I export from the cell interior to the extracellular environment, and thereby affect HMO production in multiple manners.

### Results of the Deep Well Assays

Strains were characterized in deep well assays as described in the “Materials and method section”.

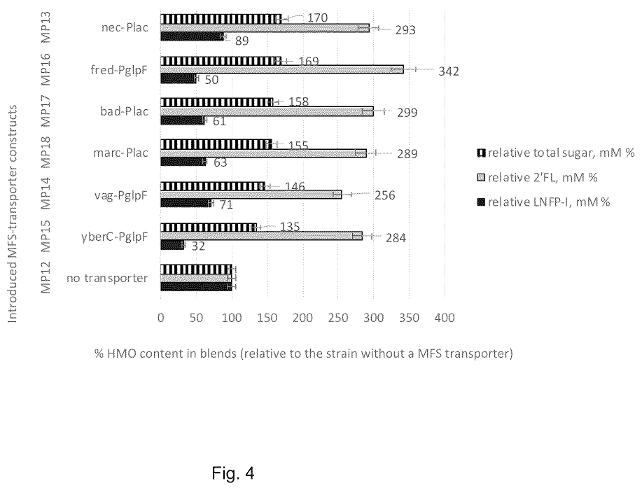

As shown in FIG. 4, the introduction of selected heterologous genes that code sugar efflux transporter proteins (Table 5) in the genetic background of futC-expressing cells resulted in a drastic change in the acquired HMO blend. It is noted that the promoter of choice to drive the expression of the cassettes bearing these transporter genes was either Plac or PglpF. However, in the present Example, data for only Plac- or PglpF-based constructs for each transporter is shown. In detail, the construct that had the most pronounced effect for each transporter was included in the data presented in FIG. 4.

In general, as shown in FIG. 4, the introduction of any of the selected MFS transporter genes clearly reduced the formation of LNFP-I and largely increased 2′-FL formation, presumably due to the efficient transporter-mediated export of 2′-FL—but not LNFP-I—out of cell. The large increase in the 2′-FL fraction in transporter-expressing cells was reflected on the total HMO concentration of the obtained blends, with the corresponding blends showing 35-70% higher HMO content relative to the blend of the host strain with no MFS transporter (MP12) (FIG. 4).

Depending on the transporter gene that was introduced into the genetic background of the production host, the LNFP-I concentration in the resulting blend varied significantly relative to the control (host) strain and represented 90%, 70%, 60%, 50%, or only 30% of the LNFP-I that was formed in the host cell that does not encode a heterologous MFS transporter. The largest reduction (70%) in LNFP-I concentration in the final blend was observed with the introduction of the PglpF-yberC construct, while minor losses (10%) in the LNFP-I content of the final blend were observed with the introduction of the Plac-nec construct.

On the contrary, the blends resulting from the introduction of transporter-constructs in the production host showed a 2.5 to 3.5-fold increase in 2′-FL concentration compared to the blend generated by cells that lack a heterologous transporter. The highest relative increase in 2′-FL concentration of a blend was obtained with the introduction of the PglpF-fred construct, while the lowest relative increase was obtained with the introduction of the PglpF-vag construct (FIG. 4).

As mentioned above, the total HMO concentration in the HMO blends that were generated by the strains expressing a heterologous sugar efflux transporter showed 35-70% higher HMO content compared to the blend of the host strain. The highest increase in HMO content relative to the host was observed with the introduction of the Plac-nec and PglpF-fred constructs, which are the ones that led to some of the highest relative increases in 2′-FL concentration as well (FIG. 4).

In conclusion, the introduction of selected heterologous genes coding sugar efflux transporter proteins of the MFS superfamily in the genetic background of futC-expressing cells can drastically inverse the order of the abundance of the first and second most abundant HMO of the final HMO blend from LNFP-I>2′-FL to 2′-FL>LNFP-I. Such genetic modifications can also lead to extensive changes in the total HMO concentrations obtained in the final blend, with the total HMO content increasing up to 70% in transporter-expressing cells, depending on the transporter-construct introduced in the LNFP-I production host.

### Example 5—Generation of Variations of HMO Blends of LNFP-I, 2′-FL and LNT by Deleting the glpR Gene that Represses PglpF-Driven Gene Expression

Description of the genotype of strains MP19 and MP20 tested in deep well assays Based on the previously reported platform strain (“MDO”), the modifications summarised in Table 6, were made to obtain the LNFP-I producing strains MP19 and MP20 used in this study, both being fully chromosomal strains. The strains are capable of producing the tetrasaccharide HMO, LNT and fucosylate LNT further to obtain the pentasaccharide HMO, LNFP-I. The fucosyltransferase enzyme used for this reaction, the FutC enzyme from Helicobacter pylori (GenBank ID: WP_080473865.1, but with two additional amino acids (LG) at the C-terminus, SEQ ID NO: 6) Notably, other HMOs, such as LNT and LNT II were present in the final HMO blends generated by the above-mentioned strains, but only at low concentrations.

In the present Example, it was demonstrated how the deletion of the glpR gene is used as a genetic tool to obtain a specific target composition of a HMO mixture comprising up to four HMOs, including LNFP-I, 2′-FL, LNT II and LNT (in order of abundance). This disclosure demonstrates how the deletion of the glpR gene can be advantageously used to modulate the composition of the HMO blend produced by strains MP19 and MP20. The only difference between the two strains, as shown in Table 6, is the knock-out of the glpR gene. The gene product of glpR is the DNA-binding transcriptional repressor GlpR, which acts as the repressor of the glycerol-3-phosphate regulon, which is organized in different operons. One of its targets is the PglpF promoter, which is originally found in front of the native E. coli gene glpF, which codes the glycerol facilitator GlpF. Since the colanic acid gene cluster and the heterologous genes coding MFS transporters or glycosyltransferases for HMO synthesis are under the control of the PglpF promoter, the deletion of the glpR gene eliminates the GlpR-imposed repression of transcription from all PglpF promoters in the cell and in this manner it can enhance gene expression from all PglpF-based cassettes that are present in the genome of the host, and thereby affect overall HMO production in multiple manners.

### Results of the Deep Well Assays

Strains were characterized in deep well assays as described in the “Materials and method section”.

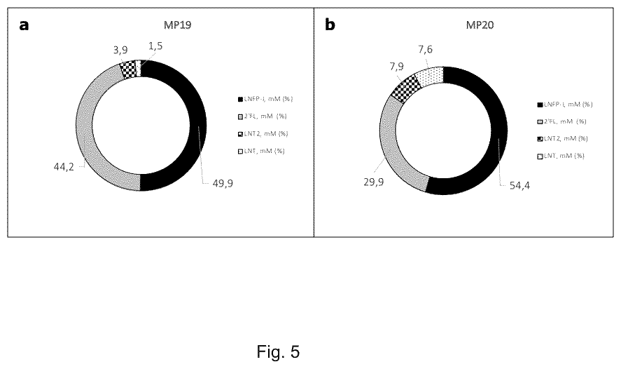

As shown in FIG. 5, a single genetic modification, namely the deletion of the glpR gene, resulted in a notable change in the distribution of HMO's in the acquired HMO blend. Deletion of the glpR gene changed markedly the formation of 2′-FL, LNT and LNT-II, while the formation of LNFP-I was marginally affected. Specifically, the glpR+strain, MP19, generated a blend consisting of 50% LNFP-I and 44% 2′-FL, while the glpR-strain, MP20, provided a blend with 15% less 2′-FL and just 5% more LNFP-I compared to MP19. Moreover, glpR deletion is seemingly associated with higher LNT and LNT-II fractions in the final HMO blend (FIG. 5).

The deletion of the glpR gene resulted in a minor loss in total HMO concentration (7%) in the blend acquired by the strain MP20 compared to the blend generated by the strain MP19, i.e., the strain MP19 produced 5.7 mM of total sugar while the strain MP20 produced 5.3 mM of HMOs (data not shown).

In conclusion, the deletion of the glpR gene changed the individual HMO abundance in the resulting blend in such a manner that the LNFP-I to 2′-FL ratio became higher (MP20) than the one in the blend of glpR+cells (MP19). This genetic modification also increased the abundance of LNT II and LNT in the resulting blend, but they both remained the least abundant sugars in the final blend.

### Example 6—Generation of Variations of HMO Blends of LNFP-I, 2′-FL and LNT by Fermentation Temperature Modulation

### Description of Genotype of the Strain Used in Fermentation

Based on the previously reported platform strain (“MDO”), the modifications summarised in Table 7, were made to obtain the LNFP-I producing strain used in this study, i.e., fully chromosomal strain MP21. The strain is capable of producing the tetrasaccharide HMO, LNT and fucosylate LNT further to obtain the pentasaccharide HMO, LNFP-I. The fucosyltransferase enzyme used for this reaction, namely the FutC enzyme α-1,2-fucosylosyl-transferase, derived from Helicobacter pylori (GenBank ID: WP_080473865.1, but with two additional amino acids (LG) at the C-terminus SEQ ID NO: 6), was found to be able to fucosylate both, LNT but also lactose as substrates, to yield, as predominant products of this strain, LNFP-I and 2′-FL, respectively. LNT will also accumulate, but to a lesser extent, leaving an HMO blend composed of LNFP-I, 2′-FL and LNT. To an even lesser extent also DFL (=2′,3-difucosyllactose) is obtained from fucosylation of 2′-FL, but only in considerable amounts if lactose availability is limited (see also example 7).

### Description of the Fermentation Process

The fermentations were carried out in 2 L fermenters bioreactors (Sartorius, Biostat B), starting with 900 mL of defined mineral culture medium, consisting of 30 g/kg carbon source (glucose), MgSO4×7H2O, KOH, H3PO4, trace element solution, citric acid, antifoam and thiamine. The trace metal solution (TMS) contained Mn, Cu, Fe, Zn as sulphate salts and citric acid. Fermentations were started by inoculation with 2% (v/v) of pre-cultures grown in a defined minimal medium. After depletion of the carbon source contained in the batch medium, a sterile feed solution containing glucose, MgSO4×7H2O, TMS and H3PO4 was fed continuously in a carbon-limited manner using a predetermined, non-linear profile.

Lactose monohydrate at 75 g/kg was added within a 30 min period, starting one hour after start of glucose feeding. The pH throughout fermentation was controlled at 6.8 by titration with 28% NH4OH solution. Aeration was at 1 vvm using air, and dissolved oxygen was controlled above 30% of air saturation. At 15 min after glucose feed start, the fermentation temperature setpoint was lowered from 33° C. to the respective setpoints under investigation, as shown in Tables 8 and 9. These temperature drops were conducted with a linear ramp over 3 hours. End-of-fermentation was at approximately 95-98 hours, when the target composition of the HMO mix and lactose had been reached.

Throughout the fermentation, samples were taken in order to determine the concentration of LNFP-I, 2′-FL, LNT, LNT II, DFL, lactose and other minor by-products using HPLC. Total broth samples were diluted three-fold in deionized water and boiled for 20 minutes. This was followed by centrifugation at 17000 g for 3 minutes, where after the resulting supernatant was analysed by HPLC. The above measurements were used to accurately calculate the ratios of each HMO relative to the sum of HMO with lactose (“HMOL”) and without lactose (“HMO”).

### Results of the Fermentation Runs

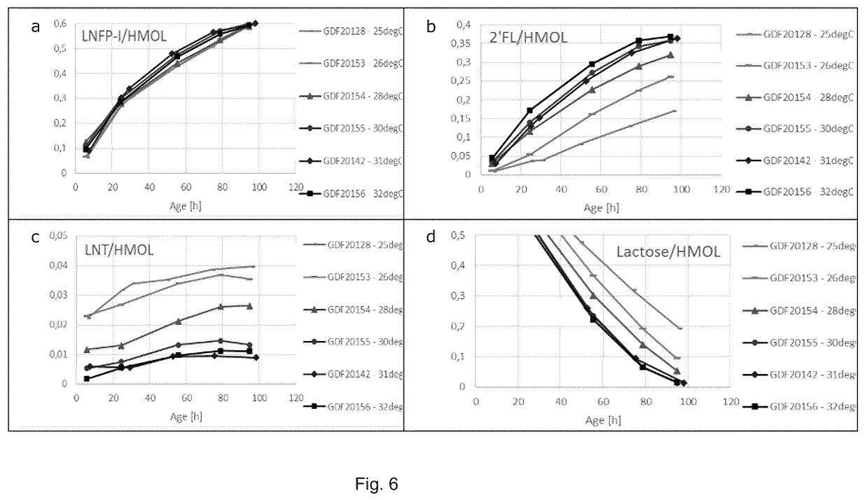

FIG. 6 depicts the development of the three major HMOs, namely LNFP-I, 2′-FL and LNT, produced by strain MP21 in fermentations at different production temperatures, and of the acceptor lactose. The graphs show the molar ratio of each individual compound divided by the sum of all HMOs and lactose (“HMOL”). Surprisingly, while LNFP-I/HMOL molar ratio increases to around 45% in all cases, 2′-FL and LNT show a highly temperature-dependent behaviour. Thus, at the end-of-fermentation, 2′-FL/HMOL molar ratio ranges from 20% to 50% while LNT/HMOL molar ratio ranges from slightly below 1% to 3.54%. Furthermore, while 2′-FL/HMOL increases proportionally with temperature in the range investigated (25-32° C.), LNT/HMOL behaves inversely proportional with temperature i.e., the lower the production temperature, the higher the LNT/HMOL molar ratio. All fermentation endpoint molar ratios are shown in Table 8. Since lactose can be as high as almost 33% relative to HMOL at the end-of-fermentation, and lower levels do not lead to a sudden change in any of the HMO/HMOL ratios, we can assume that the fermentation process can be adequately controlled in order to secure the desired composition with very low residual levels of lactose in the final fermentation sample. Besides an improved fermentation process to end at the lowest possible residual lactose levels, DSP operations, such as membrane processes, can be applied in addition to lower the lactose concentration selectively. Therefore, it is feasible to obtain an end-product with the composition ranges shown in Table 9. Hence, product ranges could be as follows: LNFP-I [47-63], 2′-FL [31-51] and LNT [1-5] relative to the sum of all HMOs, or in the combination LNFP-I/2′-FL/LNT between 63/31/5 and 47/51/1 (all in % by molar).

### Example 7—Generation of Variations of HMO Blends of LNFP-I, 2′-FL and LNT by Lactose Concentration Modulation During Fermentation

Description of Genotype of Strains MP19 and MP22 Tested in Fermentations with High or Low Lactose Process

Based on the previously reported platform strain (“MDO”), the modifications summarised in Table 10, were made to obtain the LNFP-I producing strains MP19 and MP22 used in this study, both being fully chromosomal strains. The strains are capable of producing the tetrasaccharide HMO, LNT and fucosylate LNT further to obtain the pentasaccharide HMO, LNFP-I. The fucosyltransferase enzyme used for this reaction, namely the FutC enzyme α-1,2-fucosylosyl-transferase, derived from Helicobacter pylori (GenBank ID: WP_080473865.1, but with two additional amino acids (LG) at the C-terminus, SEQ ID NO: 6), was found to be able to fucosylate LNT to yield LNFP-I as predominant product of these strains. Likewise, other HMOs are being produced with 2′-FL, LNT and LNT-II being the predominant side products at varying concentrations, depending on the growth conditions in fermentation, in particular the concentration of the acceptor lactose during fermentation. In Example 6 it was demonstrated how modulation of the lactose level during fermentation is used to obtain a specific target composition of a HMO mixture comprising up to four HMOs in significant quantities of LNFP-I, 2′-FL, LNT and LNT-II.

Therefore, this disclosure deals with how lactose addition during fermentation can be advantageously used to modulate the composition of the HMO blend produced by strains MP19 and MP22. The only difference between the two strains lies in genomic loci that were selected for the integration of the heterologous glycosyltransferases.

Description of the Fermentation Processes with High and Low Lactose Levels

The fermentations were carried out in 200 mL DasBox bioreactors (Eppendorf, Germany), starting with 100 mL of defined mineral culture medium, consisting of 30 g/kg carbon source (glucose), MgSO4×7H2O, KOH, NaOH, NH4H2PO4, KH2PO4, trace element solution, citric acid, antifoam and thiamine. The trace metal solution (TMS) contained Mn, Cu, Fe, Zn as sulphate salts and citric acid. Fermentations were started by inoculation with 2% (v/v) of pre-cultures grown in a defined minimal medium. After depletion of the carbon source contained in the batch medium, a sterile feed solution containing glucose, MgSO4×7H2O, TMS and H3PO4 was fed continuously in a carbon-limited manner using a predetermined, linear profile.

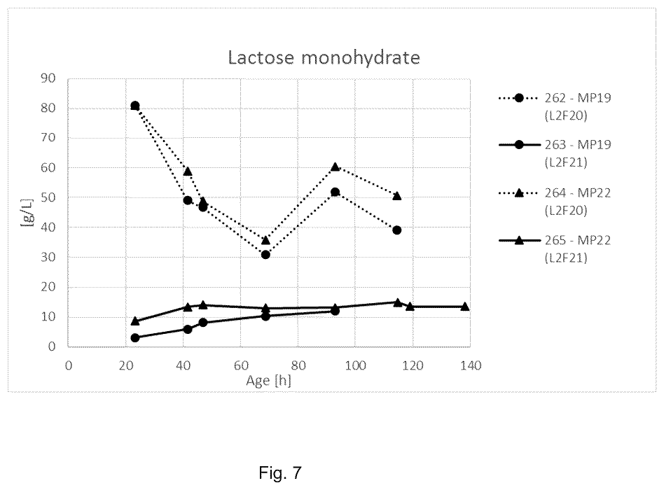

Lactose addition was done in two different ways, depending on if a high or low lactose process was chosen. In the high lactose process (“L2F20”), lactose monohydrate solution was added by two bolus additions, the first one at approx. 10 hours after feed start, the second one at approx. 70 hours EFT. In the low lactose process (“L2F21”), lactose was fed continuously as part of the glucose feed solution. As shown in FIG. 7, this resulted in the following lactose concentration ranges: high lactose process 30-80 g/L, low lactose process 0-15 g/L.

The pH throughout fermentation was controlled at 6.8 by titration with 14% NH4OH solution. Aeration was controlled at 1 vvm using air, and dissolved oxygen was kept above 23% of air saturation, controlled by the stirrer rate. At 15 min after glucose feed start, the fermentation temperature setpoint was lowered from 33° C. to 25° C. This temperature drop was conducted instantly without a ramp. Fermentations were operated until instability in terms of excessive foaming was observed.

Throughout the fermentations, samples were taken in order to determine the concentration of LNFP-I, 2′-FL, LNT, LNT-II, DFL, lactose and other minor by-products using HPLC. Total broth samples were diluted three-fold in deionized water and boiled for 20 minutes. This was followed by centrifugation at 17000 g for

3 minutes, where after the resulting supernatant was analysed by HPLC. The above measurements were used to accurately calculate the ratios of each HMO relative to the total sum of HMO (“HMO”).

### Results of the Fermentation Runs

The four fermentations ran in a stable manner for at least 68.7 h. In three instances, excessive foaming occurred late in fermentation, while GDF17265 ran in a very stable manner for 138.3 hours. For the reason of comparison, Table 11 depicts HMO compositions in fermentation samples at timepoint 68.7 h. The numbers represent ratios of the individual HMOs LNFP-I, 2′-FL, LNT and LNT-II as a ratio to the total sum of these four HMOs including DFL (“HMO”), in molar-%. DFL numbers are not shown since this HMO only appears in traces of up to 0.3 g/L. As depicted in FIG. 7, the two processes allow to maintain lactose levels of 30-80 g/L for the high lactose process L2F20 and 0-15 g/L for the low lactose process L2F21. The two fermentation processes are otherwise identical with regard to medium composition, glucose feed profile and fermentation process parameters such as temperature, pH and dissolved oxygen.

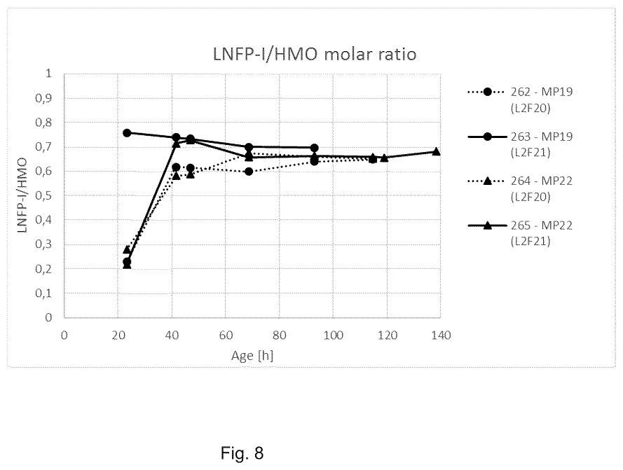

Furthermore, as depicted in FIG. 8, the predominant HMO product in all fermentations is LNFP-I in a range between 65% and 80% of the total HMO produced. This level is already reached early in fermentation, i.e., from approximately 40 hours, and remains virtually unchanged throughout. Moreover, the LNFP-I/HMO ratio is almost independent from lactose levels, i.e., only slightly higher with the low lactose process for both strains tested.

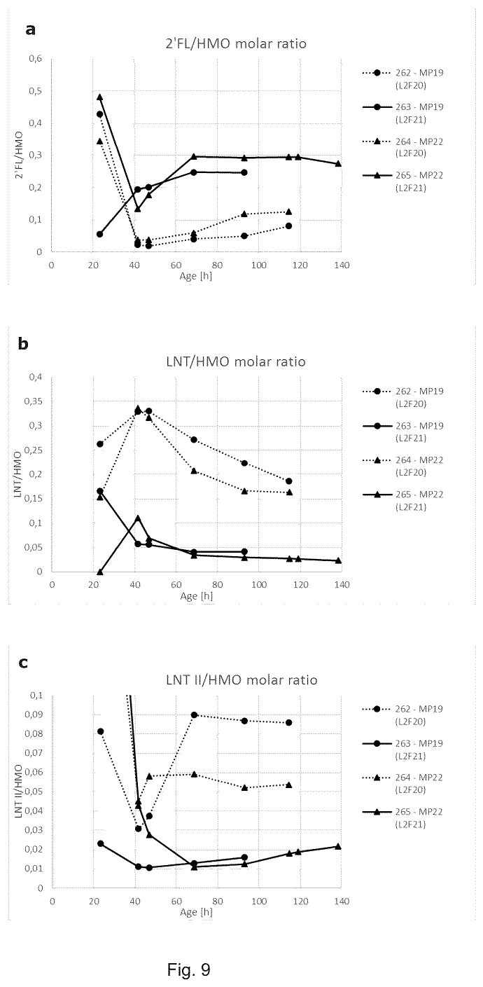

Finally, FIG. 9a-c depicts the time profiles of the three product ratios 2′-FL/HMO, LNT/HMO and LNT II/HMO in the fermentation broth throughout the four runs. The data reveal two major product compositions which are highly dependent on the lactose concentration. In the high lactose process (L2F20), the HMO product profile is (in decreasing order) LNFP-I>2′-FL>LNT>LNT II. In the low lactose process (L2F21), the HMO product profile is LNFP-I>LNT>LNT-II>=2′-FL. This applies equally to both strains investigated. Thus, as shown in Table 11, the following ranges can be found for the high lactose process in % by molar: LNFP-I [47-63], 2′-FL [31-51] and LNT [1-5] relative to the sum of all HMOs, or in the combination LNFP-I/2′-FL/LNT between 63/31/5 and 47/51/1 (all in % by molar). And the following ranges were found for the low lactose process in molar %: LNFP-I/HMO [66-70], 2′-FL/HMO [25-30], LNT/HMO [3-4], LNT-II/HMO [1-1.5].

Hence, lactose concentration can be powerful control tool to achieve a pre-determined, desired profile of 3-4 major HMOs during the production of HMO blends containing predominantly LNFP-I.

### Example 8—the Concomitant Expression of the Smob Enzyme Heterologous MFS Transporters Nec or YberC Increase LNFP-I Formation

The present Example describes an optimized strain engineering approach to construct a strain with a high LNFP-I to 2′-FL ratio, and with a significant fraction of the product being found in the supernatant of the culture.

### Description of the Genotype of Strains MP8, MP23, MP24 and MP25

Based on the previously reported platform strain (“MDO”), the modifications summarised in Table 12, were made to obtain the fully chromosomal strains MP8, MP23, MP24 and MP25. The strains can produce the pentasaccharide HMO LNFP-I. The glycosyltransferase enzymes LgtA (a β-1,3-N-acetyloglucosamine transferase) from N. meningitidis, GalTK (a β-1,3-galactosyltransferase) from H. pylori and Smob (α-1,2-fucosyltransferase) from S. mobilis are present in all four strains. Moreover, the strain MP6 expresses the heterologous transporter of the Major Facilitator Superfamily (MFS) YberC from Yersinia bercovieri, while the strains MP5 and MP7 express the heterologous MFS transporter Nec from Rosenbergiella nectarea. The only difference between the latter two strains lies in the strength of the promoter that drives the expression of the nec gene, i.e. a PglpF-driven nec copy is present in the strain MP5, while the strain MP7 expresses the nec gene under the control of the Plac promoter.

### Results of the Deep Well Assays

Strains were characterized in deep well assays as described in the “Materials and method section” with the change that a 20% lactose solution (10 ml pr 75 ml) was used. The concentration of the detected HMOs in each sample was used to calculate the % quantitative differences in the HMO content of the strains tested, i.e., the % HMO content of nec- and yberC-expressing cells relative to the HMO content of cells that do not express a heterologous transporter.

The newly formed HMO of interest needs to be exported to the cell exterior to alleviate the cell from the HMO-imposed osmotic stress. The identification of sugar exporters and the fine balancing of their expression can be a key for the success of such production systems. This task can though be challenging, since only the HMO of interest, and not the precursor or elongated versions thereof, should be bound and exported by the chosen sugar exporter.

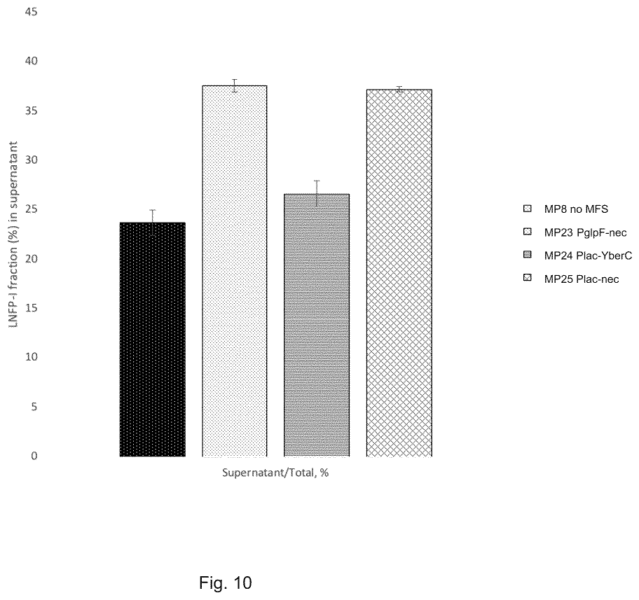

In the present example Nec and YberC sugar transporters have been shown to be able to export the LNFP-I product out of the cell. In detail, only 24% of the total LNFP-I was detected in the supernatant for cells that do not express an MFS transporter (strain MP8), while approximately 38% of the synthesized LNFP-I was detected in the supernatant of cultures for cells expressing the Nec transporter (FIG. 10).

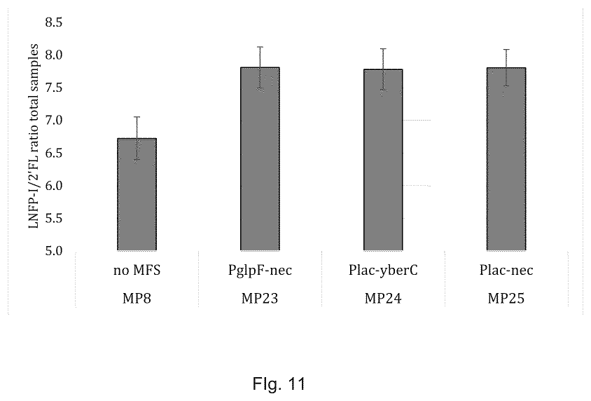

Moreover, the introduction of a nec or YberC sugar exporter in the strains induces changes in the LNFP-I to 2′-FL ratio in the HMO blend produced by the cell. Specifically, in the strains with the MFS transporter the ratio is increased from 6.7 to approximately 7.8 when compared to the strain that does not express a sugar transporter (strain MP8) (FIG. 11).

Following the approach described here, HMOs other than LNFP-I constitute only a minor fraction of the total HMO blend delivered by the engineered cell. In the framework of the present Example, introducing the heterologous genes, smob and nec or yberC, into the genome of an E. coli DH K12 strain that already produces LNT can be advantageously employed with a high copy number for the IgtA gene to deliver an efficient LNFP-I cell factory with the beneficial traits described above.

In conclusion, the balanced expression of the β-1,3-N-acetyloglucosamine transferase LgtA, the β-1,3-galactosyltransferase GalTK, the α-1,2-fucosyltransferase Smob and either of the MFS transporters Nec or YberC constitute an effective strain engineering strategy for the generation an HMO blend with a higher ratio of LNFP-I to 2′FL.

1. A method for the production of a human milk oligosaccharide (HMO) blend with 2′-FL and LNFP-I as the predominant HMO(s), the method comprising the steps of:
a. providing a genetically engineered cell capable of producing at least two HMO's, wherein said cell
i. comprises a heterologous β-1,3-N-acetyl-glucosaminyltransferase protein as shown in SEQ ID NO: 1, 2 or 3, or a functional homologue thereof having an amino acid sequence which is at least 80% identical to SEQ ID NO: 12 or 3,
ii. comprises a heterologous β-1,3-galactosyltransferase protein as shown in SEQ ID NO: 4 or 5, or a functional homologue thereof having an amino acid sequence which is at least 80% identical to SEQ ID NO: 4 or 5, and
iii. comprises a heterologous α-1,2-fucosyltransferase protein as shown in any one of SEQ ID NO: 6 and 7 and 49, or a functional homologue thereof having an amino acid sequence which is at least 80% identical to any one of SEQ ID NO: 6 or 7 or 49,
iv. expresses functionally the colanic acid gene cluster, and
v. comprises a native or heterologous regulatory element for controlling the expression of and of i)-iv),
b. culturing the cell according to (α) in a suitable cell culture medium to express said proteins and to produce an HMO blend; and
c. harvesting the human milk oligosaccharide (HMO) blend produced in step (b),

2. The method according to claim 1, wherein the colanic acid gene cluster is overexpressed by increasing the copy number and/or by choosing an appropriate element for regulatory element(s) for controlling the expression.

3. The method according to claim 1, wherein the heterologous β-1,3-N-acetyl-glucosaminyltransferase protein is SEQ ID NO: 1, or a functional homologue thereof having an amino acid sequence which is at least 80% identical to SEQ ID NO: 1.

4. The method according to claim 1, wherein the heterologous β-1,3-galactosyltransferase protein is SEQ ID NO: 4 or a functional homologue thereof having an amino acid sequence which is at least 80% identical to SEQ ID NO: 4

5. The method according to claim 1, wherein the expression of i) and ii) is overexpressed by increasing the copy number and/or by choosing an appropriate regulatory element for i) and ii).

6. The method according to claim 1, wherein the expression of i) and/or ii) is obtained from a single copy and/or the regulatory element for expression of i) and/or ii) has low or intermediate strength.

7. The method according to claim 1, wherein the expression of i) and/or ii) is obtained from two or more copies and/or the regulatory element for expression of i) and/or ii) has high strength.

8. The method according to claim 1, wherein the regulatory element is selected from any one of SEQ ID NO: 9 to 26.

9.-10. (canceled)

11. The method according to claim 1, wherein a gene product that binds to v) or regions upstream of v) and represses the expression of any one of i), ii), iii) or iv), has been deleted or made non-functional within the cell.

12. The method according to claim 11, wherein said gene product is the DNA-binding transcriptional repressor GlpR (SEQ ID NO: 48).

13. The method according to claim 1, wherein the heterologous α-1,2-fucosyltransferase protein of iii) is FutC (SEQ ID NO: 6).

14. The method according to claim 1, wherein the cell further comprises a gene product that upon expression acts as a sugar efflux transporter.

15. The method according to claim 14, wherein the amino acid sequence of the sugar efflux transporter is selected from the group consisting of
i. SEQ ID NO: 28 or a functional homologue thereof having an amino acid sequence which is at least 70% identical to SEQ ID NO: 28,
ii. SEQ ID NO: 29 or a functional homologue thereof having an amino acid sequence which is at least 70% identical to SEQ ID NO: 29,
iii. SEQ ID NO: 30 or a functional homologue thereof having an amino acid sequence which is at least 70% identical to SEQ ID NO: 30,
iv. SEQ ID NO: 31 or a functional homologue thereof having an amino acid sequence which is at least 70% identical to SEQ ID NO: 31,
v. SEQ ID NO: 32 or a functional homologue thereof having an amino acid sequence which is at least 70% identical to SEQ ID NO: 32, and
vi. SEQ ID NO: 33 or a functional homologue thereof having an amino acid sequence which is at least 70% identical to SEQ ID NO: 33.

16. The method according to claim 1, wherein the HMO blend has molar % of 2′-FL between 25% to 70% and LNFP-I between 30% to 60% of the total HMO

17. The method according to claim 1, wherein the fermentation temperature during the culturing of the genetically engineered cell in step (b) is between 3° and 32° C., and wherein the molar % of 2′-FL is between 30% and 40% of the produced blend of HMOs.

18. The method according to claim 1, wherein the level of lactose the fermentation medium during the culturing of the genetically engineered cell in step (b) is below 20 g/L, and wherein the molar % of 2′-FL is between 25% and 35% of the produced blend of HMOs.

19. A genetically engineered cell comprising a recombinant nucleic acid sequence encoding
ii. a heterologous β-1,3-N-acetyl-glucosaminyltransferase protein as shown in SEQ ID NO: 1 or 2 or 3, or a functional homologue thereof having an amino acid sequence which is at least 80% identical to SEQ ID NO: 1, 2 or 3; and
iii. a heterologous β-1,3-galactosyltransferase protein as shown in SEQ ID NO: 4 or 5, or a functional homologue thereof having an amino acid sequence which is at least 80% identical to SEQ ID NO: 4 or 5; and
iv. a heterologous α-1,2-fucosyltransferase protein as shown in any one of SEQ ID NO: 6 and 7, or a functional homologue thereof having an amino acid sequence which is at least 80% identical to any one of SEQ ID NO: 6 or 7 or 49, and
v. the colanic acid gene cluster, and
vi. a native or heterologous regulatory or episomal element for controlling the expression of any of i)-iv) and
vii. a recombinant nucleic acid sequence encoding a sugar efflux transporter capable of exporting 2′FL and/or LNFP-I out of the cell.

20. The genetically engineered cell according to claim 19, wherein colanic acid gene cluster is overexpressed by increasing the copy number, by choosing an appropriate regulatory element, or both.

21. The genetically engineered cell according to claim 19, wherein the cell is selected from the group consisting of E. coli, C. glutamicum, L. lactis, B. subtilis, S. lividans, P. pastoris, and S. cerevisiae.

22.-24. (canceled)
